SEQUENCE LISTING (12 sequences)

SEQ ID NO: 1 - protein, 26 aa, synthetic construct
  source 1..26: note = BCL9 26-mer sequence for residues 350-275
GLSQEQLEHRERSLQTLRDIQRMLFP

SEQ ID NO: 2 - protein, 25 aa, synthetic construct
  source 1..25: note = BCL9 26 mer sequence of residues 350-375 biotinylated at N-terminus
  MOD_RES 1: note = 2-aminocaproic acid biotinylated at N-terminus
XGLSQEQLEHRERSLQTLRDIQRML

SEQ ID NO: 3 - DNA, 19 bp, synthetic construct
  source 1..19: mol_type = other DNA; note = human GAPDH forward primer
gaaggtgaaggtcggagtc

SEQ ID NO: 4 - DNA, 20 bp, synthetic construct
  source 1..20: mol_type = other DNA; note = human GAPDH reverse primer
gaagatggtgatgggatttc

SEQ ID NO: 5 - DNA, 26 bp, synthetic construct
  source 1..26: mol_type = other DNA; note = human HPRT forward primer
gctataaattctttgctgacctgctg

SEQ ID NO: 6 - DNA, 28 bp, synthetic construct
  source 1..28: mol_type = other DNA; note = human HPRT reverse primer
aattacttttatgtcccctgttgactgg

SEQ ID NO: 7 - DNA, 20 bp, synthetic construct
  source 1..20: mol_type = other DNA; note = human AXIN2 forward primer
agtgtgaggtccacggaaac

SEQ ID NO: 8 - DNA, 20 bp, synthetic construct
  source 1..20: mol_type = other DNA; note = human AXIN2 reverse primer
cttcacactgcgatgcattt

SEQ ID NO: 9 - DNA, 20 bp, synthetic construct
  source 1..20: mol_type = other DNA; note = human LEF1 forward primer
gacgagatgatccccttcaa

SEQ ID NO: 10 - DNA, 20 bp, synthetic construct
  source 1..20: mol_type = other DNA; note = human LEF1 reverse primer
agggctcctgagaggtttgt

SEQ ID NO: 11 - DNA, 23 bp, synthetic construct
  source 1..23: mol_type = other DNA; note = human cyclin D1 forward primer
acaaacagatcatccgcaaacac

SEQ ID NO: 12 - DNA, 20 bp, synthetic construct
  source 1..20: mol_type = other DNA; note = human cyclin D1 reverse primer
tgttggggctcctcaggttc

(19) United States
(12) Patent Application Publication
(10) Pub. No.: US 2025/0019371 A1
(43) Pub. Date: Jan. 16, 2025

(54) SMALL-MOLECULAR INHIBITORS FOR THE BETA-CATENIN/ B-CELL LYMPHOMA 9 PROTEIN-PROTEIN INTERACTION

(71) Applicant: H. LEE MOFFITT CANCER CENTER AND RESEARCH INSTITUTE, INC., Tampa, FL (US)

(72) Inventor: Haitao JI, Tampa, FL (US)

(21) Appl. No.: 18/704,795

(22) PCT Filed: Oct. 28, 2022
(86) PCT No.: PCT/US2022/078859

§ 371 Date: Apr. 25, 2024

Related U.S. Application Data
(60) Provisional application No. 63/272,770, filed on Oct. 28, 2021.

(51) Int. Cl. C07D 409/12; A61K 31/4535; A61P 35/00

(52) U.S. Cl. CPC C07D 409/12; A61K 31/4535; A61P 35/00

(57) ABSTRACT

The present disclosure provides inhibitors of the beta-catenin/B-cell lymphoma 9 interaction as well as their use in the treatment of oncological disorders. Further provided herein are methods of treating or preventing cancer in a subject, comprising administering to the subject an effective amount of a compound or composition as disclosed herein.

### CROSS-REFERENCE TO RELATED APPLICATIONS

This application claims the benefit of priority to U.S. Provisional Application No. 63/272,770, filed Oct. 28, 2021, the disclosure of which is incorporated herein by reference in its entirety.

### TECHNICAL FIELD

This disclosure relates to compounds for use in the treatment of medical disorders, and more particularly to inhibitors of the β-catenin/B-cell lymphoma 9 protein-protein interaction and their use in the treatment of oncological disorders such as cancer.

### BACKGROUND

The Wnt/β-catenin signaling pathway participates in various biological processes including embryogenesis, tissue homeostasis, and stem cell renewal.¹ In unstimulated cells, the protein β-catenin, a central hub of this pathway, undergoes constitutive degradation guided by a destruction complex consisting of Axin, adenomatous polyposis coli (APC), glycogen synthase kinase 3β (GSK3β), casein kinase 1α (CK1α), and protein phosphatase 2A (PP2A), and thus free β-catenin are maintained at none-to-baseline levels.²⁻³ Upon activation of Wnt/β-catenin signaling by a Wnt ligand, the destruction complex disassembles and β-catenin is liberated. Free-catenin accumulates in the cytosol in its dephosphorylated state and then translocates into the cell nucleus, where it binds the T-cell factor (TCF) and lymphoid enhancer-binding factor (LEF) family of transcriptional factors, and recruits co-activators B-cell lymphoma 9 (BCL9) or BCL9-like (BCL9L), Pygopus (Pygo 1 or Pygo 2), CREB-binding protein (CBP)/p300, and among others to activate β-catenin target genes.⁴⁻⁷

Aberrant activation of the Wnt/β-catenin signaling cascade has been recorded in many cancers. For instance, APC mutations that lead to β-catenin accumulation were detected in ˜80% of colorectal cancer.⁸ Loss-of-function mutations in AXIN were observed in colorectal cancer, hepatocellular carcinoma, and oesophageal squamous cell carcinoma.⁹⁻¹¹ Oncogenic β-catenin activation mutations were described in colorectal cancer, melanoma, hepatocellular carcinoma, thyroid tumor, and ovarian endometrioid adenocarcinoma.¹²⁻¹³ The autocrine/paracrine secretion of upstream effectors and the epigenetic silencing of the genes of negative regulators of the Wnt/β-catenin pathway also activates β-catenin signaling and have been reported for various types of cancers.¹⁴⁻¹⁸ In addition, β-catenin signaling plays important roles in cancer immune evasion and immunotherapy resistance.¹⁹⁻²¹ These collective evidence strongly supports suppression of hyperactive Wnt/β-catenin signaling as a new direction for developing anticancer therapy.

The upstream effectors of the Wnt/β-catenin pathway have been extensively targeted by various therapeutic agents.²²⁻²⁷ However, these agents are expected to be ineffective against cancer cells with more downstream genetic or epigenetic mutations, such as loss-of-function mutations of APC and AXIN, or activation mutations of CTNNB1 (β-catenin target gene). Furthermore, some of these agents might cause off-pathway effects given that their targets (GSK3β, tankyrase, and CK1α) are upstream and involved in multiple cellular processes. β-Catenin/BCL9 protein-protein interaction (PPI), located in the β-catenin-containing transcriptional complex, appears to be a promising target to suppress hyperactive Wnt/β-catenin signaling. The β-catenin/BCL9 interaction is significantly upregulated in tumor tissues, and the murine gut BCL9/BCL9L elimination did not lead to overt phenotypic consequences or affect normal intestinal homeostasis, indicating targeting this PPI might have no or very low toxicity.²⁸⁻²⁹ BCL9/BCL9L knockdown by siRNAs and shRNAs significantly inhibits viability of β-catenin-dependent cancer cells in vitro and in vivo.^{28,30} BCL9/BCL9L conditional ablation in murine oncogenic intestinal organoids induced their differentiation and completely abolished their tumorigenicity, without affecting their proliferation.³¹ More importantly, BCL9/BCL9L loss blocks tumorigenesis driven by Wnt signaling and extends disease-free survival in models that recapitulate human cancer.³²⁻³³

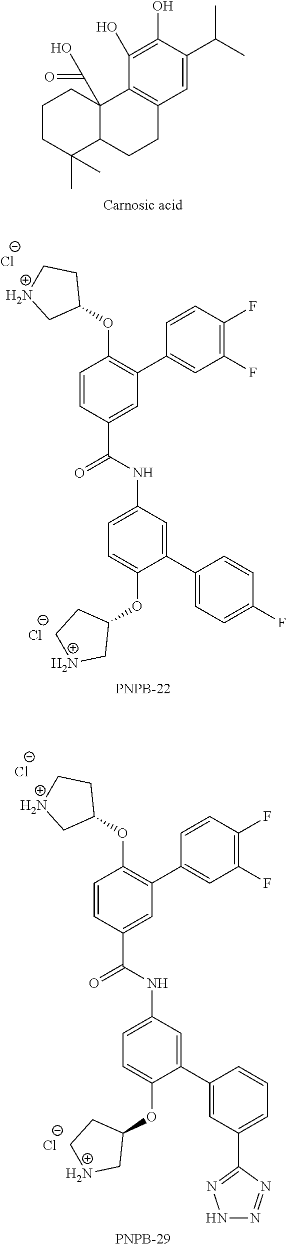

Apart from peptide-based inhibitors,³²⁻³⁵ two series of small-molecule inhibitors have been reported to disrupt the β-catenin/BCL9 PPI.³⁶⁻⁴⁰ In 2012, carnosic acid was reported as an inhibitor of the β-catenin/BCL9 PPI after screening two compound libraries by Bienz and coworkers. Recently our group reported 3-(4-fluorophenyl)-N-phenylbenzamide (PNPB) derivatives shown above as β-catenin/BCL9 inhibitors by rational design and optimization. While the existing scaffolds contain some challenges for further inhibitor optimization, it is highly desirable to discover new small-molecule inhibitors with novel scaffolds to disrupt β-catenin/BCL9 PPI for chemical biology and anti-cancer drug discovery studies.

### SUMMARY

In accordance with the purposes of the disclosed materials and methods, as embodied and broadly described herein, the disclosed subject matter, in one aspect, relates to compounds, compositions and methods of making and using compounds and compositions.

In one aspect, a compound of Formula I is provided:

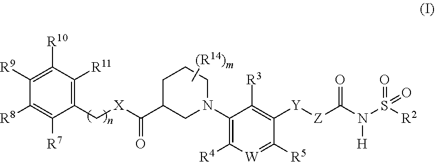

or a pharmaceutically acceptable salt thereof;
wherein all variables are as defined further herein.

The compound can have a chiral designation of R, S, or may be a racemic mixture.

Pharmaceutical compositions comprising a therapeutically effective amount of a compound as described herein or a pharmaceutically acceptable salt hereof, and a pharmaceutically acceptable carrier are also disclosed.

In specific aspects, the disclosed subject matter relates to cancer therapy and to anti-cancer compounds. More specifically, the subject matter disclosed herein relates to inhibitors for the β-catenin/B-cell lymphoma 9 interaction. Further, the subject matter disclosed herein relates to inhibitors that are selective for β-catenin/B-cell lymphoma 9 interactions over-catenin/E-cadherin PPI interaction. Also disclosed are methods of inhibiting the β-catenin/B-cell lymphoma 9 interaction, as well as methods of treating certain cancers.

In specific aspects, the disclosed subject matter relates to cancer therapy and to anti-cancer compounds. More specifically, the subject matter disclosed herein relates to degradation of β-catenin. Also disclosed are methods of degrading nuclear β-catenin as methods of treating certain cancers.

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### DESCRIPTION OF DRAWINGS

FIGS. 1A, 1B, 1C, and 1D shows the Wnt-responsive TOPFlash and FOPFlash luciferase reporter assay results of inhibitors 28 and 30 in SW480 cells.

FIGS. 2A and 2B depicts qPCR to determine changes of mRNA expression of Axin2, cyclin D1, and LEFT in response to different concentrations of 30 (24-h incubation) in Wnt 3a-stimulated MDA-MB-231 (FIG. 2A) and SW480 cells (FIG. 2B). House-keeper gene HPRT was used as the negative control. * P<0.05, ** P<0.01, as determined by the unpaired, two-tailed Student t test.

FIGS. 3A and 3B depict (FIG. 3A) Western blot to monitor changes of protein expression of Axin2 and cyclin D1 in response to different concentrations of 30 (24-h incubation) in SW480 cells. 8-Tubulin was used as an internal reference. (FIG. 3B) C₀-IP experiments to evaluate the disruption of the β-catenin/BCL9 PPI by 30 and the inhibitory selectivity of 30 for β-catenin/BCL9 over β-catenin/E-cadherin PPI using HCT116 cells. IP, immunoprecipitation; IB, immunoblotting. Each experiment was performed in duplicate Like reference symbols in the various drawings indicate like elements.

### DETAILED DESCRIPTION

The following description of the disclosure is provided as an enabling teaching of the disclosure in its best, currently known embodiments. Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As can be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It can be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a compound”, “a composition”, or “a cancer”, includes, but is not limited to, two or more such compounds, compositions, or cancers, and the like.

It should be noted that ratios, concentrations, amounts, and other numerical data can be expressed herein in a range format. It can be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it can be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “effective amount” refers to an amount that is sufficient to achieve the desired modification of a physical property of the composition or material. For example, an “effective amount” of a monomer refers to an amount that is sufficient to achieve the desired improvement in the property modulated by the formulation component, e.g. desired antioxidant release rate or viscoelasticity. The specific level in terms of wt % in a composition required as an effective amount will depend upon a variety of factors including the amount and type of monomer, amount and type of polymer, e.g., acrylamide, amount of antioxidant, and desired release kinetics.

As used herein, the term “therapeutically effective amount” refers to an amount that is sufficient to achieve the desired therapeutic result or to have an effect on undesired symptoms but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed drug delivery composition can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the term “prophylactically effective amount” refers to an amount effective for preventing onset or initiation of a disease or condition.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the terms “optional” or “optionally” means that the subsequently described event or circumstance can or cannot occur, and that the description includes instances where said event or circumstance occurs and instances where it does not.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as an ophthalmological disorder. The effect can be therapeutic in terms of a partial or complete cure of a disease, condition, symptom or adverse effect attributed to the disease, disorder, or condition. The term “treatment” as used herein can include any treatment of ophthalmological disorder in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

As used herein, “therapeutic” can refer to treating, healing, and/or ameliorating a disease, disorder, condition, or side effect, or to decreasing in the rate of advancement of a disease, disorder, condition, or side effect.

### Chemical Definitions

Compounds are described using standard nomenclature. Unless defined otherwise, all technical and scientific terms used herein have the same meaning as is commonly understood by one of skill in the art to which this invention belongs.

The compounds described herein include enantiomers, mixtures of enantiomers, diastereomers, tautomers, racemates and other isomers, such as rotamers, as if each is specifically described, unless otherwise indicated or otherwise excluded by context.

A dash (“-”) that is not between two letters or symbols is used to indicate a point of attachment for a substituent. For example, —(C═O)NH₂ is attached through the carbon of the keto (C═O) group.

The term “substituted”, as used herein, means that any one or more hydrogens on the designated atom or group is replaced with a moiety selected from the indicated group, provided that the designated atom's normal valence is not exceeded and the resulting compound is stable. For example, when the substituent is oxo (i.e., ═O) then two hydrogens on the atom are replaced. For example, a pyridyl group substituted by oxo is a pyridine. Combinations of substituents and/or variables are permissible only if such combinations result in stable compounds or useful synthetic intermediates. A stable active compound refers to a compound that can be isolated and can be formulated into a dosage form with a shelf life of at least one month. A stable manufacturing intermediate or precursor to an active compound is stable if it does not degrade within the period needed for reaction or other use. A stable moiety or substituent group is one that does not degrade, react or fall apart within the period necessary for use. Non-limiting examples of unstable moieties are those that combine heteroatoms in an unstable arrangement, as typically known and identifiable to those of skill in the art.

Any suitable group may be present on a “substituted” or “optionally substituted” position that forms a stable molecule and meets the desired purpose of the invention and includes, but is not limited to: alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, silyl, sulfo-oxo, sulfonyl, sulfone, sulfoxide, sulfonylamino, or thiol.

“Alkyl” is a straight chain or branched saturated aliphatic hydrocarbon group. In certain embodiments, the alkyl is C₁-C₂, C₁-C₃, or C₁-C₆ (i.e., the alkyl chain can be 1, 2, 3, 4, 5, or 6 carbons in length). The specified ranges as used herein indicate an alkyl group with length of each member of the range described as an independent species. For example, C₁-C₆alkyl as used herein indicates an alkyl group having from 1, 2, 3, 4, 5, or 6 carbon atoms and is intended to mean that each of these is described as an independent species and C₁-C₄alkyl as used herein indicates an alkyl group having from 1, 2, 3, or 4 carbon atoms and is intended to mean that each of these is described as an independent species. When C₀-Cₙalkyl is used herein in conjunction with another group, for example (C₃-C₇cycloalkyl) C₀-C₄alkyl, or —C₀-C₄ (C₃-C₇cycloalkyl), the indicated group, in this case cycloalkyl, is either directly bound by a single covalent bond (C₀alkyl), or attached by an alkyl chain, in this case 1, 2, 3, or 4 carbon atoms. Alkyls can also be attached via other groups such as heteroatoms, as in —O—C₀-C₄alkyl(C₃-C₇cycloalkyl). Examples of alkyl include, but are not limited to, methyl, ethyl, n-propyl, isopropyl, n-butyl, isobutyl, sec-butyl, t-butyl, n-pentyl, isopentyl, tert-pentyl, neopentyl, n-hexyl, 2-methylpentane, 3-methylpentane, 2,2-dimethylbutane, and 2,3-dimethylbutane. In one embodiments, the alkyl group is optionally substituted as described herein.

“Cycloalkyl” is a saturated mono- or multi-cyclic hydrocarbon ring system. When composed of two or more rings, the rings may be joined together in a fused or bridged fashion. Non-limiting examples of typical cycloalkyl groups include cyclopropyl, cyclobutyl, cyclopentyl, cyclohexyl, and cycloheptyl. In one embodiment, the cycloalkyl group is optionally substituted as described herein.

“Alkenyl” is a straight or branched chain aliphatic hydrocarbon group having one or more carbon-carbon double bonds, each of which is independently either cis or trans, that may occur at a stable point along the chain. Non-limiting examples include C₂-C₄alkenyl and C₂-C₆alkenyl (i.e., having 2, 3, 4, 5, or 6 carbons). The specified ranges as used herein indicate an alkenyl group having each member of the range described as an independent species, as described above for the alkyl moiety. Examples of alkenyl include, but are not limited to, ethenyl and propenyl. In one embodiment, the alkenyl group is optionally substituted as described herein.

“Alkynyl” is a straight or branched chain aliphatic hydrocarbon group having one or more carbon-carbon triple bonds that may occur at any stable point along the chain, for example, C₂-C₄alkynyl or C₂-C₆alkynyl (i.e., having 2, 3, 4, 5, or 6 carbons). The specified ranges as used herein indicate an alkynyl group having each member of the range described as an independent species, as described above for the alkyl moiety. Examples of alkynyl include, but are not limited to, ethynyl, propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, and 5-hexynyl. In one embodiment, the alkynyl group is optionally substituted as described herein.

“Alkoxy” is an alkyl group as defined above covalently bound through an oxygen bridge (—O—). Examples of alkoxy include, but are not limited to, methoxy, ethoy, n-propoxy, isopropoxy, n-butoxy, 2-butoxy, tert-butoxy, n-pentoxy, 2-pentoxy, 3-pentoxy, isopentoxy, neopentoxy, n-hexoxy, 2-hexoxy, 3-hexoxy, and 3-methylpentoxy. Similarly, an “alkylthio” or “thioalkyl” group is an alkyl group as defined above with the indicated number of carbon atoms covalently bound through a sulfur bridge (—S—). In one embodiment, the alkoxy group is optionally substituted as described herein.

“Alkanoyl” is an alkyl group as defined above covalently bound through a carbonyl(C═O) bridge. The carbonyl carbon is included in the number of carbons, for example C₂alkanoyl is a CH₃ (C═O)— group. In one embodiment, the alkanoyl group is optionally substituted as described herein.

“Haloalkoxy” indicates a haloalkyl group as defined herein attached through an oxygen bridge (oxygen of an alcohol radical).

“Halo” or “halogen” indicates, independently, any of fluoro, chloro, bromo or iodo.

“Aryl” indicates an aromatic group containing only carbon in the aromatic ring or rings. In one embodiment, the aryl group contains 1 to 3 separate or fused rings and is 6 to 14 or 18 ring atoms, without heteroatoms as ring members. When indicated, such aryl groups may be further substituted with carbon or non-carbon atoms or groups. Such substitution may include fusion to a 4- to 7- or 5- to 7-membered saturated or partially unsaturated cyclic group that optionally contains 1, 2, or 3 heteroatoms independently selected from N, O, B, P, Si and S, to form, for example, a 3,4-methylenedioxyphenyl group. Aryl groups include, for example, phenyl and naphthyl, including 1-naphthyl and 2-naphthyl. In one embodiment, aryl groups are pendant. An example of a pendant ring is a phenyl group substituted with a phenyl group. In one embodiment, the aryl group is optionally substituted as described herein.

The term “heterocycle” refers to saturated and partially saturated heteroatom-containing ring radicals, where the heteroatoms may be selected from N, O, and S. The term heterocycle includes monocyclic 3-12 members rings, as well as bicyclic 5-16 membered ring systems (which can include fused, bridged, or spiro bicyclic ring systems). It does not include rings containing —O—O—, —O—S—, and —S—S-portions. Examples of saturated heterocycle groups including saturated 4- to 7-membered monocyclic groups containing 1 to 4 nitrogen atoms [e.g., pyrrolidinyl, imidazolidinyl, piperidinyl, pyrrolinyl, azetidinyl, piperazinyl, and pyrazolidinyl]; saturated 4- to 6-membered monocyclic groups containing 1 to 2 oxygen atoms and 1 to 3 nitrogen atoms [e.g., morpholinyl]; and saturated 3- to 6-membered heteromonocyclic groups containing 1 to 2 sulfur atoms and 1 to 3 nitrogen atoms [e.g., thiazolidinyl]. Examples of partially saturated heterocycle radicals include, but are not limited, dihydrothienyl, dihydropyranyl, dihydrofuryl, and dihydrothiazolyl. Examples of partially saturated and saturated heterocycle groups include, but are not limited to, pyrrolidinyl, imidazolidinyl, piperidinyl, pyrrolinyl, pyrazolidinyl, piperazinyl, morpholinyl, tetrahydropyranyl, thiazolidinyl, dihydrothienyl, 2,3-dihydro-benzo[1,4]dioxanyl, indolinyl, isoindolinyl, dihydrobenzothienyl, dihydrobenzofuryl, isochromanyl, chromanyl, 1,2-dihydroquinolyl, 1,2,3,4-tetrahydro-isoquinolyl, 1,2,3,4-tetrahydro-quinolyl, 2,3,4,4a,9,9a-hexahydro-1H-3-aza-fluorenyl, 5,6,7-trihydro-1,2,4-triazolo[3,4-a] isoquinolyl, 3,4-dihydro-2H-benzo[1,4]oxazinyl, benzo[1,4]dioxanyl, 2,3, -dihydro-1H-benzo[d]isothazol-6-yl, dihydropyranyl, dihydrofuryl, and dihydrothiazolyl. Bicyclic heterocycle includes groups wherein the heterocyclic radical is fused with an aryl radical wherein the point of attachment is the heterocycle ring. Bicyclic heterocycle also includes heterocyclic radicals that are fused with a carbocyclic radical. Representative examples include, but are not limited to, partially unsaturated condensed heterocyclic groups containing 1 to 5 nitrogen atoms, for example indoline and isoindoline, partially unsaturated condensed heterocyclic groups containing 1 to 2 oxygen atoms and 1 to 3 nitrogen atoms, partially unsaturated condensed heterocyclic groups containing 1 to 2 sulfur atoms and 1 to 3 nitrogen atoms, and saturated condensed heterocyclic groups containing 1 to 2 oxygen or sulfur atoms.

“Heteroaryl” refers to a stable monocyclic, bicyclic, or multicyclic aromatic ring which contains from 1 to 3, or in some embodiments 1, 2, or 3 heteroatoms selected from N, O, S, B, and P (and typically selected from N, O, and S) with remaining ring atoms being carbon, or a stable bicyclic or tricyclic system containing at least one 5, 6, or 7 membered aromatic ring which contains from 1 to 3, or in some embodiments from 1 to 2, heteroatoms selected from N, O, S, B, or P, with remaining ring atoms being carbon. In one embodiments, the only heteroatom is nitrogen. In one embodiment, the only heteroatom is oxygen. In one embodiment, the only heteroatom is sulfur. Monocyclic heteroaryl groups typically have from 5 to 6 ring atoms. In some embodiments, bicyclic heteroaryl groups are 8- to 10-membered heteroaryl groups, that is groups containing 8 or 10 ring atoms in which one 5-, 6-, or 7-membered aromatic ring is fused to a second aromatic or non-aromatic ring, wherein the point of attachment is the aromatic ring. When the total number of S and O atoms in the heteroaryl group excess 1, these heteroatoms are not adjacent to one another. In one embodiment, the total number of S and O atoms in the heteroaryl group is not more than 2. In another embodiment, the total number of S and O atoms in the heteroaryl group is not more than 1. Examples of heteroaryl groups include, but are not limited to, pyridinyl, imidazolyl, imidazopyridinyl, pyrimidinyl, pyrazolyl, triazolyl, pyrazinyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, triazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl.

### Compounds of Formula I

In one aspect, a compound of Formula I is provided:

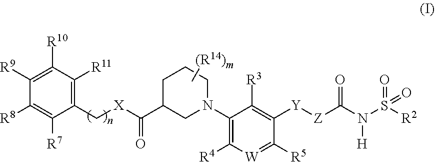

or a pharmaceutically acceptable salt thereof;
wherein:
W is CR⁶ or N;
X is selected from O or NR¹;
Y is absent or O;
Z is absent or C(R¹²)(R¹³);
m is 0, 1, or 2;
n is 1 or 2;
R¹ is selected from hydrogen, C₁-C₆alkyl, C₁-C₆haloalkyl, C₃-C₇cycloalkyl, 4- to 10-membered monocyclic or bicyclic heterocycle, 5- to 10-membered monocyclic or bicyclic aryl, 5- to 10-membered monocyclic or bicyclic heteroaryl, and R^{L}, each of which may be optionally substituted with one or more A groups as allowed by valency;
R² is selected from C₁-C₆alkyl, C₃-C₇cycloalkyl, (4- to 10-membered monocyclic and bicyclic heterocycle)-(C₀-C₀alkyl), (5- to 10-membered monocyclic or bicyclic aryl)-(C₀-C₆alkyl), (5- to 10-membered monocyclic or bicyclic heteroaryl)-(C₀-C₆alkyl), and R^{L}, each of which may be optionally substituted with one or more B groups as allowed by valency;

R³, R⁴, R⁵, and R⁶ are independently selected from hydrogen, halo, nitro, cyano, azido, C₁-C₆alkyl, C₁-C₆haloalkyl, C₂-C₆alkenyl, C₂-C₆alkynyl, C₃-C₇cycloalkyl, (4- to 10-membered monocyclic or bicyclic heterocycle)-(C₀-C₆alkyl), (5- to 10-membered monocyclic or bicyclic aryl)-(C₀-C₀alkyl), (5- to 10-membered monocyclic or bicyclic heteroaryl)-(C₀-C₆alkyl), -OR^{a}, -SR^{a}, -NR^{a}R^{b}, —C(O)R^{c}, —S(O)R^{c}, —S(O)₂R^{c}, and R^{L}, each of which may be optionally substituted with one or more A groups as allowed by valency;

R⁷, R⁸, R⁹, R¹⁰, and R¹¹ are independently selected from hydrogen, halo, nitro, cyano, azido, C₁-C₆alkyl, C₁-C₆haloalkyl, C₂-C₆alkenyl, C₂-C₆alkynyl, C₃-C₇cycloalkyl, (4- to 10-membered monocyclic or bicyclic heterocycle)-(C₀-C₀alkyl), (5- to 10-membered monocyclic or bicyclic aryl)-(C₀-C₆alkyl), (5- to 10-membered monocyclic or bicyclic heteroaryl)-(C₀-C₆alkyl), —OR^{a}, —SR^{a}, —NR^{a}R^{b}, —C(O)R^{c}, —S(O)R^{c}, —S(O) 2R^{c}, and R^{L}, each of which may be optionally substituted with one or more A groups as allowed by valency;

R12 and R₁₃ are independently selected from hydrogen, halo, nitro, cyano, azido, C₁-C₀alkyl, C₁-C₆haloalkyl, C₂-C₆alkenyl, C₂-C₆alkynyl, C₃-C₇cycloalkyl, (4- to 10-membered monocyclic or bicyclic heterocycle)-(C₀-C₆alkyl), (5- to 10-membered monocyclic or bicyclic aryl)-(C₀-C₆alkyl), (5- to 10-membered monocyclic or bicyclic heteroaryl)-(C₀-C₆alkyl), —OR^{a}, —SR^{a}, —NR^{a}R^{b}, —C(O)R^{c}, —S(O)R^{c}, —S(O) 2R^{c}, and R^{L}, each of which may be optionally substituted with one or more A groups as allowed by valency;
or R¹² and R¹³ are brought together with the carbon to which they are attached to form a C₃-C₇ cycloalkyl group or a 4- to 10-membered monocyclic or bicyclic heterocycle group;
R¹⁴ is independently selected at each occurrence from halo, nitro, cyano, azido, C₁-C₆alkyl, C₁-C₆haloalkyl, C₂-C₆alkenyl, C₂-C₆alkynyl, C₃-C₇cycloalkyl, (4- to 10-membered monocyclic or bicyclic heterocycle)-(C₀-C₆alkyl), (5- to 10-membered monocyclic or bicyclic aryl)-(C₀-C₆alkyl), (5- to 10-membered monocyclic or bicyclic heteroaryl)-(C₀-C₆alkyl), —OR^{a}, —SR^{a}, —NR^{a}R^{b}, —C(O)R^{c}, —S(O)R^{c}, —S(O)₂R^{c}, and R^{L};
R^{a} and R^{b} are independently selected at each occurrence from hydrogen, C₁-C₆alkyl, C₁-C₆haloalkyl, C₂-C₆alkenyl, C₂-C₆alkynyl, C₃-C₇cycloalkyl, (4- to 10-membered monocyclic or bicyclic heterocycle)-(C₀-C₆alkyl), (5- to 10-membered monocyclic or bicyclic aryl)-(C₀-C₆alkyl), and (5- to 10-membered monocyclic or bicyclic heteroaryl)-(C₀-C₆alkyl), each of which may be optionally substituted with one or more A groups as allowed by valency;
R^{c} is independently selected at each occurrence from hydrogen, halo, C₁-C₆alkyl, C₁-C₆haloalkyl, C₂-C₆alkenyl, C₂-C₆alkynyl, C₃-C₇cycloalkyl, (4- to 10-membered monocyclic or bicyclic heterocycle)-(C₀-C₆alkyl), (5- to 10-membered monocyclic or bicyclic aryl)-(C₁-C₆alkyl), (5- to 10-membered monocyclic or bicyclic heteroaryl)-(C₀-C₆alkyl), —OR^{a}, —SR^{a}, and —NR^{a}R^{b}, each of which may be optionally substituted with one or more A groups as allowed by valency;
A is selected at each occurrence from halo, nitro, cyano, azido, C₁-C₆alkyl, C₁-C₆haloalkyl, C₂-C₆alkenyl, C₂-C₆alkynyl, C₃-C₇cycloalkyl, (4- to 10-membered monocyclic or bicyclic heterocycle)-(C₀-C₆alkyl), (5- to 10-membered monocyclic or bicyclic aryl)-(C₀-C₆alkyl), (5- to 10-membered monocyclic or bicyclic heteroaryl)-(C₀-C₆alkyl), C₁-C₆alkoxy, C₁-C₆haloalkoxy, thiol, C₁-C₆thioalkyl, amino, (mono- or dialkyl)amino, —CHO, —COOH, —CONH₂, ester, carbamate, urea, sulfonamide, phosphate, phosphonate, and R^{L};

B is selected from halo, nitro, cyano, azido, C₁-C₆alkyl, C₁-C₆haloalkyl, C₂-C₆alkenyl, C₂-C₆alkynyl, C₃-C₇cycloalkyl, (4- to 10-membered monocyclic or bicyclic heterocycle)-(C₀-C₆alkyl), (5- to 10-membered monocyclic or bicyclic aryl)-(C₀-C₆alkyl), (5- to 10-membered monocyclic or bicyclic heteroaryl)-(C₀-C₆alkyl), —OR^{a}, —SR^{a}, —NR^{a}R^{b}, —C(O)R^{c}, —S(O)R^{c}, —S(O)₂R^{c}, and R^{L}, each of which may be optionally substituted with one or more A groups as allowed by valency; and
R^{L} is a linker conjugated to a PROTAC moiety.

In some embodiments of Formula I, W is CR⁶.

In some embodiments of Formula I, X is NR¹.

In some embodiments of Formula I, Y is O.

In some embodiments of Formula I, Z is C(R¹²) (R¹³).

In some embodiments of Formula I, m is 0. In some embodiments of Formula I, m is 1. In some embodiments of Formula I, m is 2.

In some embodiments of Formula I, n is 1. In some embodiments of Formula I, n is 2.

In some embodiments of Formula I, R¹ is hydrogen. In some embodiments of Formula I, R¹ is C₁-C₃alkyl. In some embodiments of Formula I, R¹ is C₃-C₇ cycloalkyl. In some embodiments of Formula I, R¹ is cyclopropyl.

In some embodiments of Formula I, R³ is hydrogen. In some embodiments of Formula I, R³ is halo. In some embodiments of Formula I, R³ is fluoro. In some embodiments of Formula I, R³ is chloro. In some embodiments of Formula I, R³ is haloalkyl. In some embodiments of Formula I, R³ is trifluoromethyl. In some embodiments of Formula I, R³ is cyano.

In some embodiments of Formula I, R⁴ is hydrogen. In some embodiments of Formula I, R⁴ is halo. In some embodiments of Formula I, R⁴ is fluoro. In some embodiments of Formula I, R⁴ is chloro. In some embodiments of Formula I, R³⁴ is haloalkyl. In some embodiments of Formula I, R⁴ is trifluoromethyl. In some embodiments of Formula I, R⁴ is cyano.

In some embodiments of Formula I, R⁵ is hydrogen. In some embodiments of Formula I, R³ is halo. In some embodiments of Formula I, R⁵ is fluoro. In some embodiments of Formula I, R⁵ is chloro. In some embodiments of Formula I, R⁵ is haloalkyl. In some embodiments of Formula I, R⁵ is trifluoromethyl. In some embodiments of Formula I, R⁵ is cyano.

In some embodiments of Formula I, R⁶ is hydrogen. In some embodiments of Formula I, R⁶ is halo. In some embodiments of Formula I, R⁶ is fluoro. In some embodiments of Formula I, R⁶ is chloro. In some embodiments of Formula I, R⁶ is haloalkyl. In some embodiments of Formula I, R⁶ is trifluoromethyl. In some embodiments of Formula I, R⁶ is cyano.

In some embodiments of Formula I, at least one of R³, R⁴, R⁵, and R⁶ is hydrogen. In some embodiments of Formula I, at least two of R³, R⁴, R⁵, and R⁶ are hydrogen. In some embodiments of Formula I, at least three of R³, R⁴, R⁵, and R⁶ are hydrogen. In some embodiments of Formula I, R³, R⁴, R⁵, and R⁶ are each hydrogen.

In some embodiments of Formula I, R⁷ is hydrogen. In some embodiments of Formula I, R⁷ is halo. In some embodiments of Formula I, R⁷ is fluoro. In some embodiments of Formula I, R⁷ is chloro. In some embodiments of Formula I, R⁷ is C₁-C₃alkyl. In some embodiments of Formula I, R⁷ is methyl. In some embodiments of Formula I, R⁷ is C₁-C₃haloalkyl. In some embodiments of Formula I, R⁷ is trifluoromethyl.

In some embodiments of Formula I, R⁷ is C₁-C₃alkoxy. In some embodiments of Formula I, R⁷ is methoxy. In some embodiments of Formula I, R⁷ is C₁-C₃haloalkoxy. In some embodiments of Formula I, R⁷ is trifluoromethoxy.

In some embodiments of Formula I, R⁸ is hydrogen. In some embodiments of Formula I, R⁸ is halo. In some embodiments of Formula I, R⁸ is fluoro. In some embodiments of Formula I, R⁸ is chloro. In some embodiments of Formula I, R⁸ is C₁-C₃alkyl. In some embodiments of Formula I, R⁸ is methyl. In some embodiments of Formula I, R⁸ is C₁-C₃haloalkyl. In some embodiments of Formula I, R⁸ is trifluoromethyl. In some embodiments of Formula I, R⁸ is C₁-C₃alkoxy. In some embodiments of Formula I, R⁸ is methoxy. In some embodiments of Formula I, R⁸ is C₁-C₃haloalkoxy. In some embodiments of Formula I, R⁸ is trifluoromethoxy.

In some embodiments of Formula I, R¹⁰ is hydrogen. In some embodiments of Formula I, R¹⁰ is halo. In some embodiments of Formula I, R¹⁰ is fluoro. In some embodiments of Formula I, R¹⁰ is chloro. In some embodiments of Formula I, R¹⁰ is C₁-C₃alkyl. In some embodiments of Formula I, R¹⁰ is methyl. In some embodiments of Formula I, R¹⁰ is C₁-C₃haloalkyl. In some embodiments of Formula I, R¹⁰ is trifluoromethyl. In some embodiments of Formula I, R¹⁰ is C₁-C₃alkoxy. In some embodiments of Formula I, R¹⁰ is methoxy. In some embodiments of Formula I, R¹⁰ is C₁-C₃haloalkoxy. In some embodiments of Formula I, R¹⁰ is trifluoromethoxy.

In some embodiments of Formula I, R¹¹ is hydrogen. In some embodiments of Formula I, R¹¹ is halo. In some embodiments of Formula I, R¹¹ is fluoro. In some embodiments of Formula I, R¹¹ is chloro. In some embodiments of Formula I, R¹¹ is C₁-C₃alkyl. In some embodiments of Formula I, R¹¹ is methyl. In some embodiments of Formula I, R¹¹ is C₁-C₃haloalkyl. In some embodiments of Formula I, R¹¹ is trifluoromethyl. In some embodiments of Formula I, R¹¹ is C₁-C₃alkoxy. In some embodiments of Formula I, R¹¹ is methoxy. In some embodiments of Formula I, R¹¹ is C₁-C₃haloalkoxy. In some embodiments of Formula I, R¹¹ is trifluoromethoxy.

In some embodiments of Formula I, at least one of R⁷, R⁸, R⁹, or R¹⁰ is hydrogen. In some embodiments of Formula I, at least two of R⁷, R⁸, R⁹, or R¹⁰ are hydrogen. In some embodiments of Formula I, at least three of R⁷, R⁸, R⁹, or R¹⁰ are hydrogen. In some embodiments of Formula I, R⁷, R⁸, R⁹, and R¹⁰ are each hydrogen.

In some embodiments of Formula I, R⁹ is 5- to 10-membered monocyclic or bicyclic heteroaryl.

In some embodiments of Formula I, R¹² and R₁₃ are independently selected from hydrogen and C₁-C₃alkyl. In some embodiments of Formula I, R¹² and R₁₃ are each independently C₁-C₃alkyl. In some embodiments of Formula I, R¹² and R₁₃ are brought together with the carbon to which they are attached to form a C₃-C₇cycloalkyl group.

In some embodiments, the compound of Formula I is of Formula I-a:

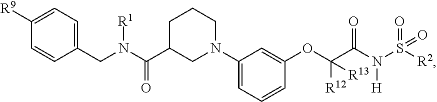

or a pharmaceutically acceptable salt thereof;
wherein all variables are as defined herein.

In some embodiments, the compound of Formula I is of Formula I-b:

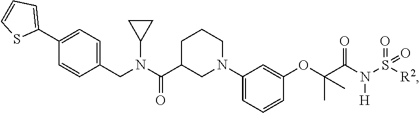

or a pharmaceutically acceptable salt thereof;
wherein R² is as defined herein.

In some embodiments of Formula I, R² is C₁-C₆ alkyl. In some embodiments of Formula I, R² is C₃-C₇cycloalkyl. In some embodiments of Formula I, R² is phenyl optionally substituted with one or more groups selected from hydroxyl, cyano, or C₁-C₆alkoxy.

In some embodiments, the compound of Formula I is selected from the group consisting of:

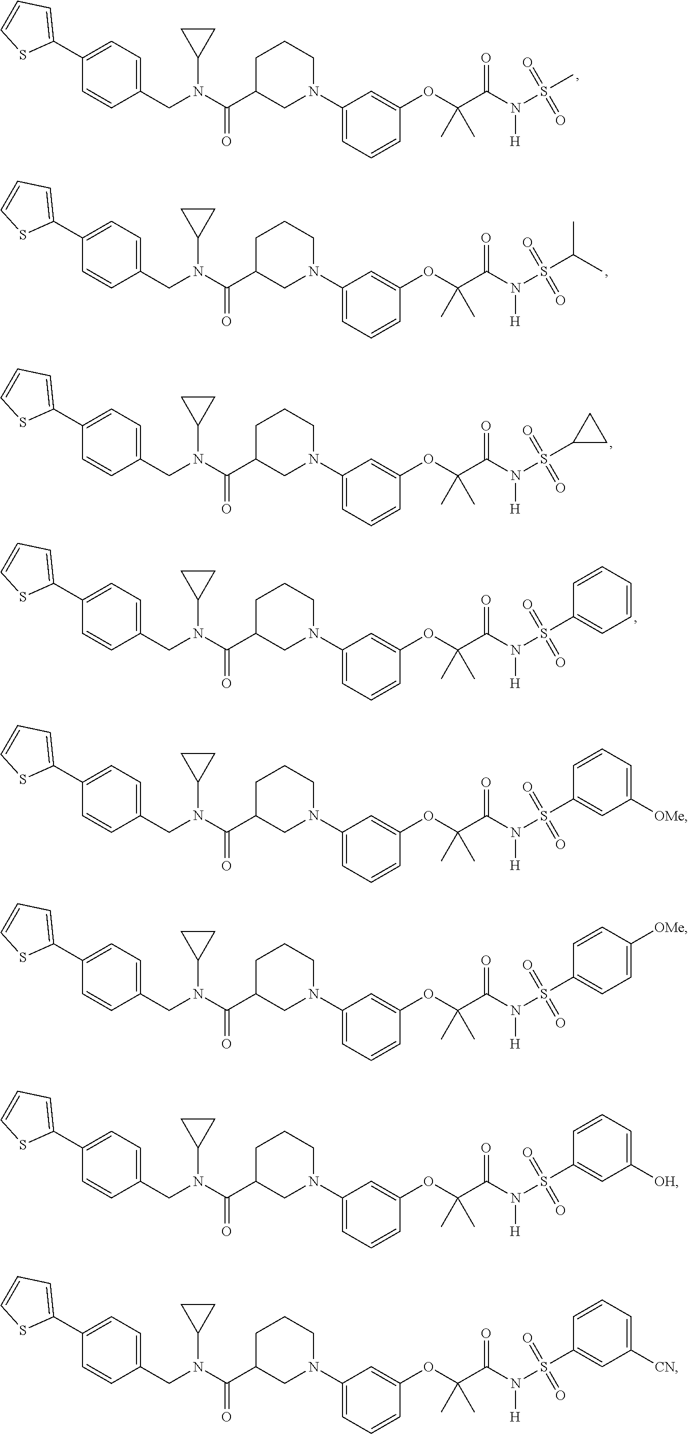

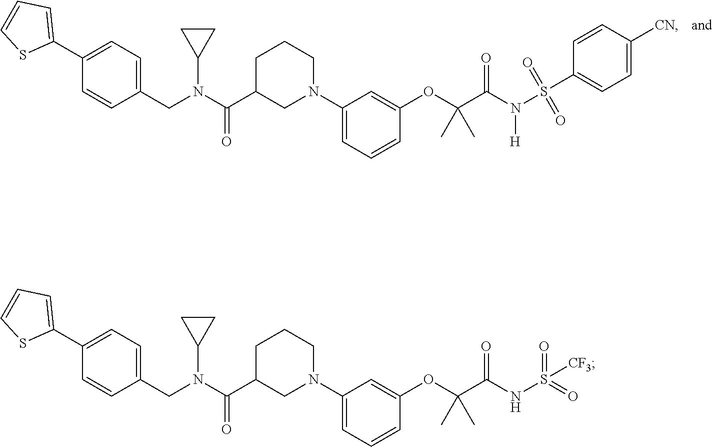 [SMILES: CC(C)(Oc1cccc(N2CCCC(C(=O)N(Cc3ccc(-c4cccs4)cc3)C3CC3)C2)c1)C(=O)NS(=O)(=O)c1ccc(C#N)cc1 ; "and" ; CC(C)(Oc1cccc(N2CCCC(C(=O)N(Cc3ccc(-c4cccs4)cc3)C3CC3)C2)c1)C(=O)NS(=O)(=O)C(F)(F)F]

or a pharmaceutically acceptable salt thereof.

In some embodiments, the compound of Formula I is selected from the group consisting of:

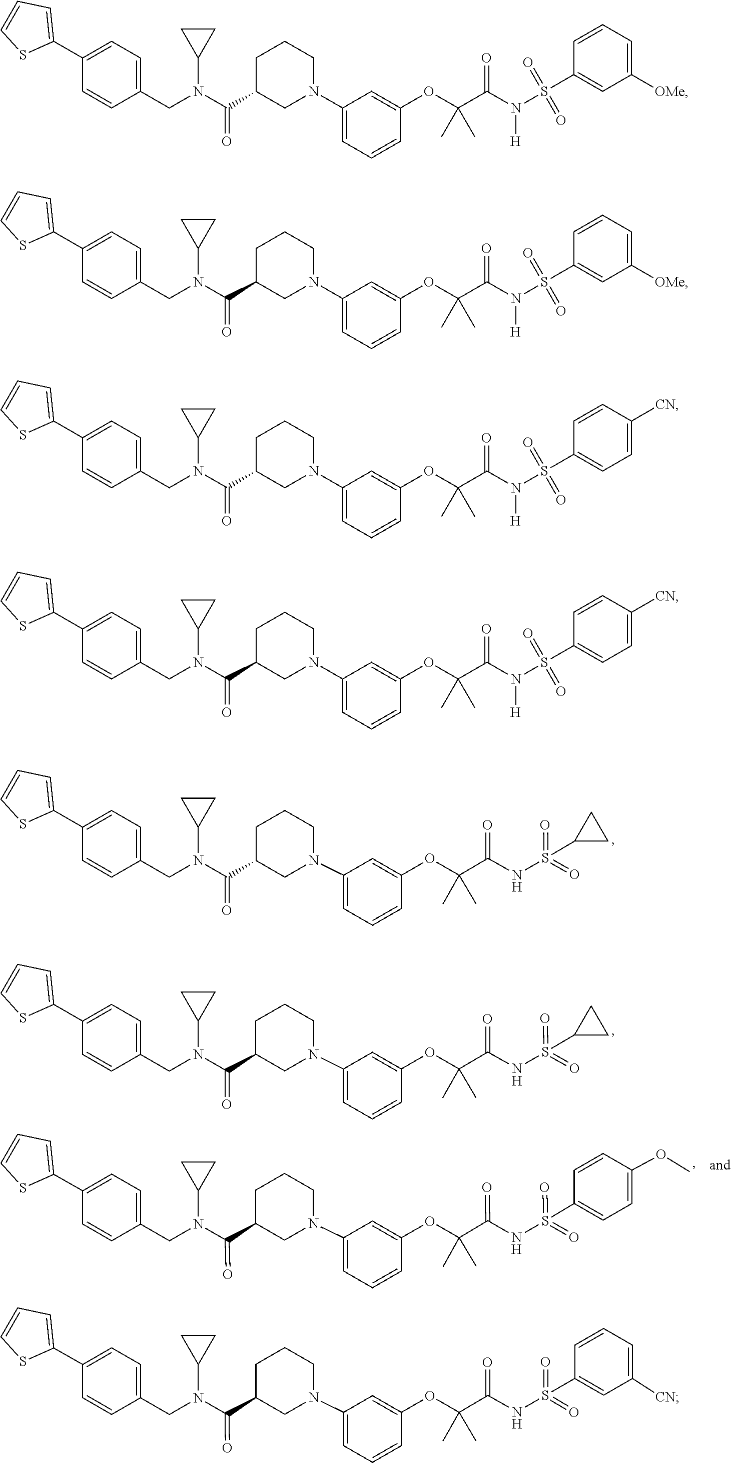

or a pharmaceutically acceptable salt thereof.

As described herein, the compounds can include a proteolysis-targeting chimera (also known as PROTAC). PROTACs are hetero-bifunctional molecules that recruit an E3 ubiquitin ligase to a given substrate protein resulting in its targeted degradation. In some embodiments, PROTACS couple a small molecule binder of a target protein to an E3 ubiquitin ligase-recruiting moiety via an intervening chemical linker. High-affinity small molecules for E3 ubiquitin ligases, in particular against von Hippel-Lindau include VH032 and against cereblon include pomalidomide.

In some examples, the linker conjugated to the PROTAC moiety comprises a linker selected from a substituted or unsubstituted C₄-C₂₄alkyl or a C₄-C₂₄alkoxy. In some examples, the PROTAC moiety is selected from a cereblon binder (such as thalidomide, lenalidomide, or pomalidomide), or a von Hippel-Lindau E3 ligase (VHL) ligand (such as VH032 or VH298). In some embodiments of Formula I, the compounds comprise at least one linker conjugated to a PROTAC moiety.

As described herein, the compound can have a chiral designation of R of S or in some cases, is a racemic mixture.

A “pharmaceutically acceptable salt” is a derivative of the disclosed compound in which the parent compound is modified by making inorganic and organic, pharmaceutically acceptable, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include salts which are acceptable for human consumption and the quaternary ammonium salts of the parent compound formed, for example, from inorganic or organic salts. Example of such salts include, but are not limited to, those derived from inorganic acids such as hydrochloric, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, mesylic, esylic, besylic, sulfanilic, 2-acetoxybenzoic, fumaric, toluenesulfonic, methanesulfone, ethane disulfonic, oxalic, isethionic, HOOC-(CH₂)₁₋₄-COOH, and the like, or using a different acid that produced the same counterion. Lists of additional suitable salts may be found, e.g., in Remington's Pharmaceutical Sciences, 17^{th} ed., Mack Publishing Company, Easton, PA., p. 1418 (1985).

The present disclosure also includes compounds of Formula I with at least one desired isotopic substitution of an atom, at an amount above the natural abundance of the isotope, i.e., enriched.

Examples of isotopes that can be incorporated into compounds of the present disclosure include isotopes of hydrogen, carbon, nitrogen, oxygen, phosphorous, fluorine, and chlorine, such as ²H, ³H, ¹¹C, ¹³C, ¹⁵N, ¹⁷O, ¹⁸O, ¹⁸F, ³¹P, ³²P, ³⁵S, ³⁶Cl, and ¹²⁵I, respectively. In one embodiment, isotopically labeled compounds can be used in metabolic studies (with ¹⁴C), reaction kinetic studies (with, for example ²H or ³H), detection or imaging techniques, such as positron emission tomography (PET) or single-photon emission computed tomography (SPECT) including drug and substrate tissue distribution assays, or in radioactive treatment of patients. In particular, an ¹⁸F labeled compound may be particularly desirable for PET or SPECT studies. Isotopically labeled compounds of this invention and prodrugs thereof can generally be prepared by carrying out the procedures disclosed herein by substituting a readily available isotopically labeled reagent for a non-isotopically labeled reagent.

By way of general example and without limitation, isotopes of hydrogen, for example deuterium (²H) and tritium (³H) may optionally be used anywhere in described structures that achieves the desired result. Alternatively or in addition, isotopes of carbon, e.g., ¹³C and ¹⁴C, may be used. In one embodiment, the isotopic substitution is replacing hydrogen with a deuterium at one or more locations on the molecule to improve the performance of the molecule as a drug, for example, the pharmacodynamics, pharmacokinetics, biodistribution, half-life, stability, AUC, Tₘₐₓ, Cₘₐₓ, etc. For example, the deuterium can be bound to carbon in allocation of bond breakage during metabolism (an alpha-deuterium kinetic isotope effect) or next to or near the site of bond breakage (a beta-deuterium kinetic isotope effect).

Isotopic substitutions, for example deuterium substitutions, can be partial or complete. Partial deuterium substitution means that at least one hydrogen is substituted with deuterium. In certain embodiments, the isotope is 80, 85, 90, 95, or 99% or more enriched in an isotope at any location of interest. In some embodiments, deuterium is 80, 85, 90, 95, or 99% enriched at a desired location. Unless otherwise stated, the enrichment at any point is above natural abundance, and in an embodiment is enough to alter a detectable property of the compounds as a drug in a human.

The compounds of the present disclosure may form a solvate with solvents (including water). Therefore, in one embodiment, the invention includes a solvated form of the active compound. The term “solvate” refers to a molecular complex of a compound of the present invention (including a salt thereof) with one or more solvent molecules. Non-limiting examples of solvents are water, ethanol, dimethyl sulfoxide, acetone and other common organic solvents. The term “hydrate” refers to a molecular complex comprising a disclosed compound and water. Pharmaceutically acceptable solvates in accordance with the invention include those wherein the solvent of crystallization may be isotopically substituted, e.g., D₂O, d6-acetone, or d6-DMSO. A solvate can be in a liquid or solid form.

A “prodrug” as used herein means a compound which when administered to a host in vivo is converted into a parent drug. As used herein, the term “parent drug” means any of the presently described compounds herein. Prodrugs can be used to achieve any desired effect, including to enhance properties of the parent drug or to improve the pharmaceutic or pharmacokinetic properties of the parent, including to increase the half-life of the drug in vivo. Prodrug strategies provide choices in modulating the conditions for in vivo generation of the parent drug. Non-limiting examples of prodrug strategies include covalent attachment of removable groups, or removable portions of groups, for example, but not limited to, acylating, phosphorylation, phosphonylation, phosphoramidate derivatives, amidation, reduction, oxidation, esterification, alkylation, other carboxy derivatives, sulfoxy or sulfone derivatives, carbonylation, or anhydrides, among others. In certain embodiments, the prodrug renders the parent compound more lipophilic. In certain embodiments, a prodrug can be provided that has several prodrug moieties in a linear, branched, or cyclic manner. For example, non-limiting embodiments include the use of a divalent linker moiety such as a dicarboxylic acid, amino acid, diamine, hydroxycarboxylic acid, hydroxyamine, di-hydroxy compound, or other compound that has at least two functional groups that can link the parent compound with another prodrug moiety, and is typically biodegradable in vivo. In some embodiments, 2, 3, 4, or 5 prodrug biodegradable moieties are covalently bound in a sequence, branched, or cyclic fashion to the parent compound. Non-limiting examples of prodrugs according to the present disclosure are formed with: a carboxylic acid on the parent drug and a hydroxylated prodrug moiety to form an ester; a carboxylic acid on the parent drug and an amine prodrug to form an amide; an amino on the parent drug and a carboxylic acid prodrug moiety to form an amide; an amino on the parent drug and a sulfonic acid to form a sulfonamide; a sulfonic acid on the parent drug and an amino on the prodrug moiety to form a sulfonamide; a hydroxyl group on the parent drug and a carboxylic acid on the prodrug moiety to form an ester; a hydroxyl on the parent drug and a hydroxylated prodrug moiety to form an ester; a phosphonate on the parent drug and a hydroxylated prodrug moiety to form a phosphonate ester; a phosphoric acid on the parent drug and a hydroxylated prodrug moiety to form a phosphate ester; a hydroxyl on the parent drug and a phosphonate on the prodrug to form a phosphonate ester; a hydroxyl on the parent drug and a phosphoric acid prodrug moiety to form a phosphate ester; a carboxylic acid on the parent drug and a prodrug of the structure HO—(CH₂)₂—O—(C₂₋₂₄ alkyl) to form an ester; a carboxylic acid on the parent drug and a prodrug of the structure HO-(CH₂)₂-S-(C₂₋₂₄ alkyl) to form a thioester; a hydroxyl on the parent drug and a prodrug of the structure HO-(CH₂)₂—O—(C₂₋₂₄ alkyl) to form an ether; a hydroxyl on the parent drug and a prodrug of the structure HO-(CH₂)₂—O—(C₂₋₂₄ alkyl) to form an thioether; and a carboxylic acid, oxime, hydrazide, hydrazine, amine or hydroxyl on the parent compound and a prodrug moiety that is a biodegradable polymer or oligomer including but not limited to polylactic acid, polylactide-co-glycolide, polyglycolide, polyethylene glycol, polyanhydride, polyester, polyamide, or a peptide.

In some embodiments, a prodrug is provided by attaching a natural or non-natural amino acid to an appropriate functional moiety on the parent compound, for example, oxygen, nitrogen, or sulfur, and typically oxygen or nitrogen, usually in a manner such that the amino acid is cleaved in vivo to provide the parent drug. The amino acid can be used alone or covalently linked (straight, branched or cyclic) to one or more other prodrug moieties to modify the parent drug to achieve the desired performance, such as increased half-life, lipophilicity, or other drug delivery or pharmacokinetic properties. The amino acid can be any compound with an amino group and a carboxylic acid, which includes an aliphatic amino acid, alkyl amino acid, aromatic amino acid, heteroaliphatic amino acid, heteroalkyl amino acid, heterocyclic amino acid, or heteroaryl amino acid.

### Methods of Treatment

Further provided herein are methods of treating or preventing cancer in a subject, comprising administering to the subject an effective amount of a compound or composition as disclosed herein. The methods can further comprise administering a second compound or composition, such as, for example, anticancer agents or anti-inflammatory agents. Additionally, the method can further comprise administering an effective amount of ionizing radiation to the subject.

Methods of killing a tumor cell are also provided herein. The methods comprise contacting a tumor cell with an effective amount of a compound or composition as disclosed herein. in some embodiments, the compounds disclosed herein can inhibit β-catenin/B-cell lymphoma 9 complex. The methods can further include administering a second compound or composition (e.g., an anticancer agent or an anti-inflammatory agent) or administering an effective amount of ionizing radiation to the subject.

Also provided herein are methods of radiotherapy of tumors, comprising contacting the tumor with an effective amount of a compound or composition as disclosed herein and irradiating the tumor with an effective amount of ionizing radiation.

Also disclosed are methods for treating oncological disorders in a patient. In one embodiment, an effective amount of one or more compounds or compositions disclosed herein is administered to a patient having an oncological disorder and who is in need of treatment thereof. The disclosed methods can optionally include identifying a patient who is or can be in need of treatment of an oncological disorder. The patient can be a human or other mammal, such as a primate (monkey, chimpanzee, ape, etc.), dog, cat, cow, pig, or horse, or other animals having an oncological disorder.

The term “neoplasia” or “cancer” is used throughout this disclosure to refer to the pathological process that results in the formation and growth of a cancerous or malignant neoplasm, i.e., abnormal tissue (solid) or cells (non-solid) that grow by cellular proliferation, often more rapidly than normal and continues to grow after the stimuli that initiated the new growth cease. Malignant neoplasms show partial or complete lack of structural organization and functional coordination with the normal tissue and most invade surrounding tissues, can metastasize to several sites, are likely to recur after attempted removal and may cause the death of the patient unless adequately treated. As used herein, the term neoplasia is used to describe all cancerous disease states and embraces or encompasses the pathological process associated with malignant, hematogenous, ascitic and solid tumors. The cancers which may be treated by the compositions disclosed herein may comprise carcinomas, sarcomas, lymphomas, leukemias, germ cell tumors, or blastomas.

Carcinomas which may be treated by the compositions of the present disclosure include, but are not limited to, acinar carcinoma, acinous carcinoma, alveolar adenocarcinoma, carcinoma adenomatosum, adenocarcinoma, carcinoma of adrenal cortex, alveolar carcinoma, alveolar cell carcinoma, basal cell carcinoma, carcinoma basocellular, basaloid carcinoma, basosquamous cell carcinoma, breast carcinoma, bronchioalveolar carcinoma, bronchiolar carcinoma, cerebriform carcinoma, cholangiocellular carcinoma, chorionic carcinoma, colloid carcinoma, comedocarcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epibulbar carcinoma, epidermoid carcinoma, carcinoma epitheliate adenoids, carcinoma exulcere, carcinoma fibrosum, gelatinform carcinoma, gelatinous carcinoma, giant cell carcinoma, gigantocellulare, glandular carcinoma, granulose cell carcinoma, hair matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, lentivular carcinoma, carcinoma lenticulare, lipomatous carcinoma, lymphoepithelial carcinoma, carcinoma mastotoids, carcinoma medullare, medullary carcinoma, carcinoma melanodes, melanotonic carcinoma, mucinous carcinoma, carcinoma muciparum, carcinoma mucocullare, mucoepidermoid carcinoma, mucous carcinoma, carcinoma myxomatodes, masopharyngeal carcinoma, carcinoma nigrum, oat cell carcinoma, carcinoma ossificans, osteroid carcinoma, ovarian carcinoma, papillary carcinoma, periportal carcinoma, preinvasive carcinoma, prostate carcinoma, renal cell carcinoma of kidney, reserve cell carcinoma, carcinoma sarcomatodes, scheinderian carcinoma, scirrhous carcinoma, carcinoma scrota, signet-ring cell carcinoma, carcinoma simplex, small cell carcinoma, solandoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberrosum, tuberous carcinoma, verrucous carcinoma, and carcinoma vilosum.

Representative sarcomas which may be treated by the compositions of the present disclosure include, but are not limited to, liposarcomas (including myxoid liposarcomas and pleomorphic liposarcomas), leiomyosarcomas, rhabdomyosarcomas, neurofibrosarcomas, malignant peripheral nerve sheath tumors, Ewing's tumors (including Ewing's sarcoma of bone, extraskeletal or non-bone) and primitive neuroectodermal tumors (PNET), synovial sarcoma, hemangioendothelioma, fibrosarcoma, desmoids tumors, dermatofibrosarcoma protuberance (DFSP), malignant fibrous histiocytoma (MFH), hemangiopericytoma, malignant mesenchymoma, alveolar soft-part sarcoma, epithelioid sarcoma, clear cell sarcoma, desmoplastic small cell tumor, gastrointestinal stromal tumor (GIST) and osteosarcoma (also known as osteogenic sarcoma) skeletal and extra-skeletal, and chondrosarcoma.

The compositions of the present disclosure may be used in the treatment of a lymphoma. Lymphomas which may be treated include mature B cell neoplasms, mature T cell and natural killer (NK) cell neoplasms, precursor lymphoid neoplasms, Hodgkin lymphomas, and immunodeficiency-associated lymphoproliferative disorders. Representative mature B cell neoplasms include, but are not limited to, B-cell chronic lymphocytic leukemia/small cell lymphoma, B-cell prolymphocytic leukemia, lymphoplasmacytic lymphoma (such as Waldenström macroglobulinemia), splenic marginal zone lymphoma, hairy cell leukemia, plasma cell neoplasms (such as plasma cell myeloma/multiple myeloma, plasmacytoma, monoclonal immunoglobulin deposition diseases, and heavy chain diseases), extranodal marginal zone B cell lymphoma (MALT lymphoma), nodal marginal zone B cell lymphoma, follicular lymphoma, primary cutaneous follicular center lymphoma, mantle cell lymphoma, diffuse large B cell lymphoma, diffuse large B-cell lymphoma associated with chronic inflammation, Epstein-Barr virus-positive DLBCL of the elderly, lyphomatoid granulomatosis, primary mediastinal (thymic) large B-cell lymphoma, intravascular large B-cell lymphoma, ALK+ large B-cell lymphoma, plasmablastic lymphoma, primary effusion lymphoma, large B-cell lymphoma arising in HHV8-associated multicentric Castleman's disease, and Burkitt lymphoma/leukemia. Representative mature T cell and NK cell neoplasms include, but are not limited to, T-cell prolymphocytic leukemia, T-cell large granular lymphocyte leukemia, aggressive NK cell leukemia, adult T-cell leukemia/lymphoma, extranodal NK/T-cell lymphoma, nasal type, enteropathy-associated T-cell lymphoma, hepatosplenic T-cell lymphoma, blastic NK cell lymphoma, lycosis fungoides/Sezary syndrome, primary cutaneous CD30-positive T cell lymphoproliferative disorders (such as primary cutaneous anaplastic large cell lymphoma and lymphomatoid papulosis), peripheral T-cell lymphoma not otherwise specified, angioimmunoblastic T cell lymphoma, and anaplastic large cell lymphoma. Representative precursor lymphoid neoplasms include B-lymphoblastic leukemia/lymphoma not otherwise specified, B-lymphoblastic leukemia/lymphoma with recurrent genetic abnormalities, or T-lymphoblastic leukemia/lymphoma. Representative Hodgkin lymphomas include classical Hodgkin lymphomas, mixed cellularity Hodgkin lymphoma, lymphocyte-rich Hodgkin lymphoma, and nodular lymphocyte-predominant Hodgkin lymphoma.

The compositions of the present disclosure may be used in the treatment of a Leukemia. Representative examples of leukemias include, but are not limited to, acute lymphoblastic leukemia (ALL), chronic lymphocytic leukemia (CLL), acute myelogenous leukemia (AML), chronic myelogenous leukemia (CML), hairy cell leukemia (HCL), T-cell prolymphocytic leukemia, adult T-cell leukemia, clonal eosinophilias, and transient myeloproliferative disease.

The compositions of the present disclosure may be used in the treatment of a germ cell tumor, for example germinomatous (such as germinoma, dysgerminoma, and seminoma), non germinomatous (such as embryonal carcinoma, endodermal sinus tumor, choriocarcinoma, teratoma, polyembryoma, and gonadoblastoma) and mixed tumors.

The compositions of the present disclosure may be used in the treatment of blastomas, for example hepatoblastoma, medulloblastoma, nephroblastoma, neuroblastoma, pancreatoblastoma, pleuropulmonary blastoma, retinoblastoma, and glioblastoma multiforme.

Representative cancers which may be treated include, but are not limited to: bone and muscle sarcomas such as chondrosarcoma, Ewing's sarcoma, malignant fibrous histiocytoma of bone/osteosarcoma, osteosarcoma, rhabdomyosarcoma, and heart cancer; brain and nervous system cancers such as astrocytoma, brainstem glioma, pilocytic astrocytoma, ependymoma, primitive neuroectodermal tumor, cerebellar astrocytoma, cerebral astrocytoma, glioma, medulloblastoma, neuroblastoma, oligodendroglioma, pineal astrocytoma, pituitary adenoma, and visual pathway and hypothalamic glioma; breast cancers including invasive lobular carcinoma, tubular carcinoma, invasive cribriform carcinoma, medullary carcinoma, male breast cancer, Phyllodes tumor, and inflammatory breast cancer; endocrine system cancers such as adrenocortical carcinoma, islet cell carcinoma, multiple endocrine neoplasia syndrome, parathyroid cancer, phemochromocytoma, thyroid cancer, and Merkel cell carcinoma; eye cancers including uveal melanoma and retinoblastoma; gastrointestinal cancers such as anal cancer, appendix cancer, cholangiocarcinoma, gastrointestinal carcinoid tumors, colon cancer, extrahepatic bile duct cancer, gallbladder cancer, gastric cancer, gastrointestinal stromal tumor, hepatocellular cancer, pancreatic cancer, and rectal cancer; genitourinary and gynecologic cancers such as bladder cancer, cervical cancer, endometrial cancer, extragonadal germ cell tumor, ovarian cancer, ovarian epithelial cancer, ovarian germ cell tumor, penile cancer, renal cell carcinoma, renal pelvis and ureter transitional cell cancer, prostate cancer, testicular cancer, gestational trophoblastic tumor, urethral cancer, uterine sarcoma, vaginal cancer, vulvar cancer, and Wilms tumor; head and neck cancers such as esophageal cancer, head and neck cancer, nasopharyngeal carcinoma, oral cancer, oropharyngeal cancer, paranasal sinus and nasal cavity cancer, pharyngeal cancer, salivary gland cancer, and hypopharyngeal cancer; hematopoietic cancers such as acute biphenotypic leukemia, acute eosinophilic leukemia, acute lymphoblastic leukemia, acute myeloid leukemia, acute myeloid dendritic cell leukemia, AIDS-related lymphoma, anaplastic large cell lymphoma, angioimmunoblastic T-cell lymphoma, B-cell prolymphocytic leukemia, Burkitt's lymphoma, chronic lymphocytic leukemia, chronic myelogenous leukemia, cutaneous T-cell lymphoma, diffuse large B-cell lymphoma, follicular lymphoma, hairy cell leukemia, hepatosplenic T-cell lymphoma, Hodgkin's lymphoma, hairy cell leukemia, intravascular large B-cell lymphoma, large granular lymphocytic leukemia, lymphoplasmacytic lymphoma, lymphomatoid granulomatosis, mantle cell lymphoma, marginal zone B-cell lymphoma, Mast cell leukemia, mediastinal large B cell lymphoma, multiple myeloma/plasma cell neoplasm, myelodysplastic syndroms, mucosa-associated lymphoid tissue lymphoma, mycosis fungoides, nodal marginal zone B cell lymphoma, non-Hodgkin lymphoma, precursor B lymphoblastic leukemia, primary central nervous system lymphoma, primary cutaneous follicular lymphoma, primary cutaneous immunocytoma, primary effusion lymphoma, plasmablastic lymphoma, Sezary syndrome, splenic marginal zone lymphoma, and T-cell prolymphocytic leukemia; skin cancers such as basal cell carcinoma, squamous cell carcinoma, skin adnexal tumors (such as sebaceous carcinoma), melanoma, Merkel cell carcinoma, sarcomas of primary cutaneous origin (such as dermatofibrosarcoma protuberans), and lymphomas of primary cutaneous origin (such as mycosis fungoides); thoracic and respiratory cancers such as bronchial adenomas/carcinoids, small cell lung cancer, mesothelioma, non-small cell lung cancer, pleuropulmonary blastoma, laryngeal cancer, and thymoma or thymic carcinoma; HIV/AIDS-related cancers such as Kaposi sarcoma; epithelioid hemangioendothelioma; desmoplastic small round cell tumor; and liposarcoma.

In some aspect, disclosed are methods for treating a tumor or tumor metastases in a subject by the administration to the subject a combination of at least one compound or composition as disclosed herein and at least one cancer immunotherapeutic agent. The disclosed compounds can be administered alone or in combination with a cancer immunotherapeutic agent. The subject can receive the therapeutic compositions prior to, during or after surgical intervention to remove all or part of a tumor. Administration may be accomplished via direct immersion; systemic or localized intravenous (i.v.), intraperitoneal (i.p.), subcutaneous (s.c.), intramuscular (i.m.), or direct injection into a tumor mass; and/or by oral administration of the appropriate formulations.

### Methods of Administration

The disclosed compounds can be administered either sequentially or simultaneously in separate or combined pharmaceutical formulations. When one or more of the disclosed compounds is used in combination with a second therapeutic agent the dose of each compound can be either the same as or differ from that when the compound is used alone. Appropriate doses will be readily appreciated by those skilled in the art.

The term “administration” and variants thereof (e.g., “administering” a compound) in reference to a compound of the invention means introducing the compound or a prodrug of the compound into the system of the animal in need of treatment. When a compound of the invention or prodrug thereof is provided in combination with one or more other active agents (e.g., a cytotoxic agent, etc.), “administration” and its variants are each understood to include concurrent and sequential introduction of the compound or prodrug thereof and other agents.

In vivo application of the disclosed compounds, and compositions containing them, can be accomplished by any suitable method and technique presently or prospectively known to those skilled in the art. For example, the disclosed compounds can be formulated in a physiologically- or pharmaceutically-acceptable form and administered by any suitable route known in the art including, for example, oral, nasal, rectal, topical, and parenteral routes of administration. As used herein, the term parenteral includes subcutaneous, intradermal, intravenous, intramuscular, intraperitoneal, and intrasternal administration, such as by injection. Administration of the disclosed compounds or compositions can be a single administration, or at continuous or distinct intervals as can be readily determined by a person skilled in the art.

The compounds disclosed herein, and compositions comprising them, can also be administered utilizing liposome technology, slow release capsules, implantable pumps, and biodegradable containers. These delivery methods can, advantageously, provide a uniform dosage over an extended period of time. The compounds can also be administered in their salt derivative forms or crystalline forms.

The compounds disclosed herein can be formulated according to known methods for preparing pharmaceutically acceptable compositions. Formulations are described in detail in a number of sources which are well known and readily available to those skilled in the art. For example, Remington's Pharmaceutical Science by E. W. Martin (1995) describes formulations that can be used in connection with the disclosed methods. In general, the compounds disclosed herein can be formulated such that an effective amount of the compound is combined with a suitable carrier in order to facilitate effective administration of the compound. The compositions used can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and therapeutic application. The compositions also preferably include conventional pharmaceutically-acceptable carriers and diluents which are known to those skilled in the art. Examples of carriers or diluents for use with the compounds include ethanol, dimethyl sulfoxide, glycerol, alumina, starch, saline, and equivalent carriers and diluents. To provide for the administration of such dosages for the desired therapeutic treatment, compositions disclosed herein can advantageously comprise between about 0.1% and 99%, and especially, 1 and 15% by weight of the total of one or more of the subject compounds based on the weight of the total composition including carrier or diluent.

“Pharmaceutically acceptable excipient” refers to an excipient that is conventionally useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

A “pharmaceutically acceptable carrier” is a carrier, such as a solvent, suspending agent or vehicle, for delivering the disclosed compounds to the patient. The carrier can be liquid or solid and is selected with the planned manner of administration in mind. Liposomes are also a pharmaceutical carrier. As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated.

Formulations suitable for administration include, for example, aqueous sterile injection solutions, which can contain antioxidants, buffers, bacteriostats, and solutes that render the formulation isotonic with the blood of the intended recipient; and aqueous and nonaqueous sterile suspensions, which can include suspending agents and thickening agents. The formulations can be presented in unit-dose or multi-dose containers, for example sealed ampoules and vials, and can be stored in a freeze dried (lyophilized) condition requiring only the condition of the sterile liquid carrier, for example, water for injections, prior to use. Extemporaneous injection solutions and suspensions can be prepared from sterile powder, granules, tablets, etc. It should be understood that in addition to the ingredients particularly mentioned above, the compositions disclosed herein can include other agents conventional in the art having regard to the type of formulation in question.

Compounds disclosed herein, and compositions comprising them, can be delivered to a cell either through direct contact with the cell or via a carrier means. Carrier means for delivering compounds and compositions to cells are known in the art and include, for example, encapsulating the composition in a liposome moiety. Another means for delivery of compounds and compositions disclosed herein to a cell comprises attaching the compounds to a protein or nucleic acid that is targeted for delivery to the target cell. U.S. Pat. No. 6,960,648 and U.S. Application Publication Nos. 20030032594 and 20020120100 disclose amino acid sequences that can be coupled to another composition and that allows the composition to be translocated across biological membranes. U.S. Application Publication No. 20020035243 also describes compositions for transporting biological moieties across cell membranes for intracellular delivery. Compounds can also be incorporated into polymers, examples of which include poly(D-L lactide-co-glycolide) polymer for intracranial tumors; poly [bis(p-carboxyphenoxy) propane: sebacic acid] in a 20:80 molar ratio (as used in GLIADEL); chondroitin; chitin; and chitosan.

For the treatment of oncological disorders, the compounds disclosed herein can be administered to a patient in need of treatment in combination with other antitumor or anticancer substances and/or with radiation and/or photodynamic therapy and/or with surgical treatment to remove a tumor. These other substances or treatments can be given at the same as or at different times from the compounds disclosed herein. For example, the compounds disclosed herein can be used in combination with mitotic inhibitors such as taxol or vinblastine, alkylating agents such as cyclophosamide or ifosfamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, antiestrogens such as tamoxifen, and/or other anti-cancer drugs or antibodies, such as, for example, GLEEVEC (Novartis Pharmaceuticals Corporation) and HERCEPTIN (Genentech, Inc.), respectively.

Many tumors and cancers have viral genome present in the tumor or cancer cells. For example, Epstein-Barr Virus (EBV) is associated with a number of mammalian malignancies. The compounds disclosed herein can also be used alone or in combination with anticancer or antiviral agents, such as ganciclovir, azidothymidine (AZT), lamivudine (3TC), etc., to treat patients infected with a virus that can cause cellular transformation and/or to treat patients having a tumor or cancer that is associated with the presence of viral genome in the cells. The compounds disclosed herein can also be used in combination with viral based treatments of oncologic disease. For example, the compounds can be used with mutant herpes simplex virus in the treatment of non-small cell lung cancer (Toyoizumi, et al., “Combined therapy with chemotherapeutic agents and herpes simplex virus type IICP34.5 mutant (HSV-1716) in human non-small cell lung cancer,” Human Gene Therapy, 1999, 10 (18): 17).

Therapeutic application of compounds and/or compositions containing them can be accomplished by any suitable therapeutic method and technique presently or prospectively known to those skilled in the art. Further, compounds and compositions disclosed herein have use as starting materials or intermediates for the preparation of other useful compounds and compositions.

Compounds and compositions disclosed herein can be locally administered at one or more anatomical sites, such as sites of unwanted cell growth (such as a tumor site or benign skin growth, e.g., injected or topically applied to the tumor or skin growth), optionally in combination with a pharmaceutically acceptable carrier such as an inert diluent. Compounds and compositions disclosed herein can be systemically administered, such as intravenously or orally, optionally in combination with a pharmaceutically acceptable carrier such as an inert diluent, or an assimilable edible carrier for oral delivery. They can be enclosed in hard or soft shell gelatin capsules, can be compressed into tablets, or can be incorporated directly with the food of the patient's diet. For oral therapeutic administration, the active compound can be combined with one or more excipients and used in the form of ingestible tablets, buccal tablets, troches, capsules, elixirs, suspensions, syrups, wafers, aerosol sprays, and the like.

The tablets, troches, pills, capsules, and the like can also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring can be added. When the unit dosage form is a capsule, it can contain, in addition to materials of the above type, a liquid carrier, such as a vegetable oil or a polyethylene glycol. Various other materials can be present as coatings or to otherwise modify the physical form of the solid unit dosage form. For instance, tablets, pills, or capsules can be coated with gelatin, wax, shellac, or sugar and the like. A syrup or elixir can contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound can be incorporated into sustained-release preparations and devices.

Compounds and compositions disclosed herein, including pharmaceutically acceptable salts, hydrates, or analogs thereof, can be administered intravenously, intramuscularly, or intraperitoneally by infusion or injection. Solutions of the active agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the active ingredient, which are adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various other antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the inclusion of agents that delay absorption, for example, aluminum monostearate and gelatin.

Sterile injectable solutions are prepared by incorporating a compound and/or agent disclosed herein in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, compounds and agents disclosed herein can be applied in as a liquid or solid. However, it will generally be desirable to administer them topically to the skin as compositions, in combination with a dermatologically acceptable carrier, which can be a solid or a liquid. Compounds and agents and compositions disclosed herein can be applied topically to a subject's skin to reduce the size (and can include complete removal) of malignant or benign growths, or to treat an infection site. Compounds and agents disclosed herein can be applied directly to the growth or infection site. Preferably, the compounds and agents are applied to the growth or infection site in a formulation such as an ointment, cream, lotion, solution, tincture, or the like. Drug delivery systems for delivery of pharmacological substances to dermal lesions can also be used, such as that described in U.S. Pat. No. 5,167,649.

Useful solid carriers include finely divided solids such as talc, clay, microcrystalline cellulose, silica, alumina and the like. Useful liquid carriers include water, alcohols or glycols or water-alcohol/glycol blends, in which the compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers, for example.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user. Examples of useful dermatological compositions which can be used to deliver a compound to the skin are disclosed in U.S. Pat. Nos. 4,608,392; 4,992,478; 4,559,157; and 4,820,508.

Useful dosages of the compounds and agents and pharmaceutical compositions disclosed herein can be determined by comparing their in vitro activity, and in vivo activity in animal models. Methods for the extrapolation of effective dosages in mice, and other animals, to humans are known to the art; for example, see U.S. Pat. No. 4,938,949.

Also disclosed are pharmaceutical compositions that comprise a compound disclosed herein in combination with a pharmaceutically acceptable carrier. Pharmaceutical compositions adapted for oral, topical or parenteral administration, comprising an amount of a compound constitute a preferred aspect. The dose administered to a patient, particularly a human, should be sufficient to achieve a therapeutic response in the patient over a reasonable time frame, without lethal toxicity, and preferably causing no more than an acceptable level of side effects or morbidity. One skilled in the art will recognize that dosage will depend upon a variety of factors including the condition (health) of the subject, the body weight of the subject, kind of concurrent treatment, if any, frequency of treatment, therapeutic ratio, as well as the severity and stage of the pathological condition.

For the treatment of oncological disorders, compounds and agents and compositions disclosed herein can be administered to a patient in need of treatment prior to, subsequent to, or in combination with other antitumor or anticancer agents or substances (e.g., chemotherapeutic agents, immunotherapeutic agents, radiotherapeutic agents, cytotoxic agents, etc.) and/or with radiation therapy and/or with surgical treatment to remove a tumor. For example, compounds and agents and compositions disclosed herein can be used in methods of treating cancer wherein the patient is to be treated or is or has been treated with mitotic inhibitors such as taxol or vinblastine, alkylating agents such as cyclophosamide or ifosfamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, antiestrogens such as tamoxifen, and/or other anti-cancer drugs or antibodies, such as, for example, GLEEVEC (Novartis Pharmaceuticals Corporation) and HERCEPTIN (Genentech, Inc.), respectively. These other substances or radiation treatments can be given at the same as or at different times from the compounds disclosed herein. Examples of other suitable chemotherapeutic agents include, but are not limited to, altretamine, bleomycin, bortezomib (VELCADE), busulphan, calcium folinate, capecitabine, carboplatin, carmustine, chlorambucil, cisplatin, cladribine, crisantaspase, cyclophosphamide, cytarabine, dacarbazine, dactinomycin, daunorubicin, docetaxel, doxorubicin, epirubicin, etoposide, fludarabine, fluorouracil, gefitinib (IRESSA), gemcitabine, hydroxyurea, idarubicin, ifosfamide, imatinib (GLEEVEC), irinotecan, liposomal doxorubicin, lomustine, melphalan, mercaptopurine, methotrexate, mitomycin, mitoxantrone, oxaliplatin, paclitaxel, pentostatin, procarbazine, raltitrexed, streptozocin, tegafur-uracil, temozolomide, thiotepa, tioguanine/thioguanine, topotecan, treosulfan, vinblastine, vincristine, vindesine, vinorelbine. In an exemplified embodiment, the chemotherapeutic agent is melphalan. Examples of suitable immunotherapeutic agents include, but are not limited to, alemtuzumab, cetuximab (ERBITUX), gemtuzumab, iodine 131 tositumomab, rituximab, trastuzamab (HERCEPTIN). Cytotoxic agents include, for example, radioactive isotopes (e.g., I¹³¹, I¹²⁵, Y⁹⁰, P³², etc.), and toxins of bacterial, fungal, plant, or animal origin (e.g., ricin, botulinum toxin, anthrax toxin, aflatoxin, jellyfish venoms (e.g., box jellyfish), etc.) Also disclosed are methods for treating an oncological disorder comprising administering an effective amount of a compound and/or agent disclosed herein prior to, subsequent to, and/or in combination with administration of a chemotherapeutic agent, an immunotherapeutic agent, a radiotherapeutic agent, or radiotherapy.

### Kits

Kits for practicing the methods of the invention are further provided. By “kit” is intended any manufacture (e.g., a package or a container) comprising at least one reagent, e.g., anyone of the compounds described herein. The kit may be promoted, distributed, or sold as a unit for performing the methods of the present invention. Additionally, the kits may contain a package insert describing the kit and methods for its use. Any or all of the kit reagents may be provided within containers that protect them from the external environment, such as in sealed containers or pouches.

To provide for the administration of such dosages for the desired therapeutic treatment, in some embodiments, pharmaceutical compositions disclosed herein can comprise between about 0.1% and 45%, and especially, 1 and 15%, by weight of the total of one or more of the compounds based on the weight of the total composition including carrier or diluents. Illustratively, dosage levels of the administered active ingredients can be: intravenous, 0.01 to about 20 mg/kg; intraperitoneal, 0.01 to about 100 mg/kg; subcutaneous, 0.01 to about 100 mg/kg; intramuscular, 0.01 to about 100 mg/kg; orally 0.01 to about 200 mg/kg, and preferably about 1 to 100 mg/kg; intranasal instillation, 0.01 to about 20 mg/kg; and aerosol, 0.01 to about 20 mg/kg of animal (body) weight.

Also disclosed are kits that comprise a composition comprising a compound disclosed herein in one or more containers. The disclosed kits can optionally include pharmaceutically acceptable carriers and/or diluents. In one embodiment, a kit includes one or more other components, adjuncts, or adjuvants as described herein. In another embodiment, a kit includes one or more anti-cancer agents, such as those agents described herein. In one embodiment, a kit includes instructions or packaging materials that describe how to administer a compound or composition of the kit. Containers of the kit can be of any suitable material, e.g., glass, plastic, metal, etc., and of any suitable size, shape, or configuration. In one embodiment, a compound and/or agent disclosed herein is provided in the kit as a solid, such as a tablet, pill, or powder form. In another embodiment, a compound and/or agent disclosed herein is provided in the kit as a liquid or solution. In one embodiment, the kit comprises an ampoule or syringe containing a compound and/or agent disclosed herein in liquid or solution form.

The following embodiments of the present disclosure are also provided:

Embodiment 1. A compound of Formula I:

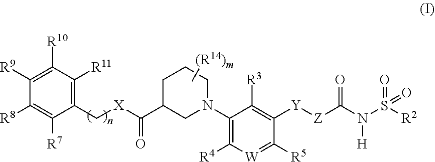

or a pharmaceutically acceptable salt thereof;
wherein:
W is CR⁶ or N;
X is selected from O or NR¹;
Y is absent or O;
Z is absent or C(R¹²) (R¹³);
m is 0, 1, or 2;
n is 1 or 2;
R¹ is selected from hydrogen, C₁-C₆alkyl, C₁-C₆haloalkyl, C₃-C₇cycloalkyl, 4- to 10-membered monocyclic or bicyclic heterocycle, 5- to 10-membered monocyclic or bicyclic aryl, 5- to 10-membered monocyclic or bicyclic heteroaryl, and R^{L}, each of which may be optionally substituted with one or more A groups as allowed by valency;
R² is selected from C₁-C₆alkyl, C₃-C₇cycloalkyl, (4- to 10-membered monocyclic and bicyclic heterocycle)-(C₀-C₆alkyl), (5- to 10-membered monocyclic or bicyclic aryl)-(C₀-C₆alkyl), (5- to 10-membered monocyclic or bicyclic heteroaryl)-(C₀-C₆alkyl), and R^{L}, each of which may be optionally substituted with one or more B groups as allowed by valency;
R³, R⁴, R⁵, and R⁶ are independently selected from hydrogen, halo, nitro, cyano, azido, C₁-C₆alkyl, C₁-C₆haloalkyl, C₂-C₆alkenyl, C₂-C₆alkynyl, C₃-C₇cycloalkyl, (4- to 10-membered monocyclic or bicyclic heterocycle)-(C₀-C₆alkyl), (5- to 10-membered monocyclic or bicyclic aryl)-(C₀-C₆alkyl), (5- to 10-membered monocyclic or bicyclic heteroaryl)-(C₀-C₆alkyl), —OR^{a}, —SR^{a}, —NR^{a}R^{b}, —C(O)R^{c}, —S(O)R^{c}, —S(O)₂R^{c}, and R^{L}, each of which may be optionally substituted with one or more A groups as allowed by valency;
R⁷, R⁸, R⁹, R¹⁰, and R¹¹ are independently selected from hydrogen, halo, nitro, cyano, azido, C₁-C₆alkyl, C₁-C₆haloalkyl, C₂-C₆alkenyl, C₂-C₆alkynyl, C₃-C₇cycloalkyl, (4- to 10-membered monocyclic or bicyclic heterocycle)-(C₀-C₆alkyl), (5- to 10-membered monocyclic or bicyclic aryl)-(C₀-C₆alkyl), (5- to 10-membered monocyclic or bicyclic heteroaryl)-(C₀-C₆alkyl), —OR^{a}, —SR^{a}, —NR^{a}R^{b}, —C(O)R^{c}, —S(O)R^{c}, —S(O)₂R^{c}, and R^{L}, each of which may be optionally substituted with one or more A groups as allowed by valency;
R¹² and R₁₃ are independently selected from hydrogen, halo, nitro, cyano, azido, C₁-C₀alkyl, C₁-C₆haloalkyl, C₂-C₆alkenyl, C₂-C₆alkynyl, C₃-C₇cycloalkyl, (4- to 10-membered monocyclic or bicyclic heterocycle)-(C₀-C₆alkyl), (5- to 10-membered monocyclic or bicyclic aryl)-(C₀-C₆alkyl), (5- to 10-membered monocyclic or bicyclic heteroaryl)-(C₀-C₆alkyl), —OR^{a}, —SR^{a}, —NR^{a}R^{b}, —C(O)R^{c}, —S(O)R^{c}, —S(O)₂R^{c}, and R^{L}, each of which may be optionally substituted with one or more A groups as allowed by valency;
or R¹² and R₁₃ are brought together with the carbon to which they are attached to form a C₃-C₇ cycloalkyl group or a 4- to 10-membered monocyclic or bicyclic heterocycle group;
R¹⁴ is independently selected at each occurrence from halo, nitro, cyano, azido, C₁-C₀alkyl, C₁-C₆haloalkyl, C₂-C₆alkenyl, C₂-C₆alkynyl, C₃-C₇cycloalkyl, (4- to 10-membered monocyclic or bicyclic heterocycle)-(C₀-C₆alkyl), (5- to 10-membered monocyclic or bicyclic aryl)-(C₀-C₆alkyl), (5- to 10-membered monocyclic or bicyclic heteroaryl)-(C₀-C₆alkyl), —OR^{a}, —SR^{a}, —NR^{a}R^{b}, —C(O)R^{c}, —S(O)R^{c}, —S(O)₂R^{c} and R¹;
R^{a} and R^{b} are independently selected at each occurrence from hydrogen, C₁-C₆alkyl, C₁-C₆haloalkyl, C₂-C₆alkenyl, C₂-C₆alkynyl, C₃-C₇cycloalkyl, (4- to 10-membered monocyclic or bicyclic heterocycle)-(C₀-C₆alkyl), (5- to 10-membered monocyclic or bicyclic aryl)-(C₀-C₆alkyl), and (5- to 10-membered monocyclic or bicyclic heteroaryl)-(C₀-C₆alkyl), each of which may be optionally substituted with one or more A groups as allowed by valency;
R^{c} is independently selected at each occurrence from hydrogen, halo, C₁-C₆alkyl, C₁-C₆haloalkyl, C₂-C₆alkenyl, C₂-C₆alkynyl, C₃-C₇cycloalkyl, (4- to 10-membered monocyclic or bicyclic heterocycle)-(C₀-C₆alkyl), (5- to 10-membered monocyclic or bicyclic aryl)-(C₁-C₆alkyl), (5- to 10-membered monocyclic or bicyclic heteroaryl)-(C₀-C₆alkyl), —OR^{a}, —SR^{a}, and —NR^{a}R^{b}, each of which may be optionally substituted with one or more A groups as allowed by valency;
A is selected at each occurrence from halo, nitro, cyano, azido, C₁-C₆alkyl, C₁-C₆haloalkyl, C₂-C₆alkenyl, C₂-C₆alkynyl, C₃-C₇cycloalkyl, (4- to 10-membered monocyclic or bicyclic heterocycle)-(C₀-C₆alkyl), (5- to 10-membered monocyclic or bicyclic aryl)-(C₀-C₆alkyl), (5- to 10-membered monocyclic or bicyclic heteroaryl)-(C₀-C₆alkyl), C₁-C₆alkoxy, C₁-C₆haloalkoxy, thiol, C₁-C₆thioalkyl, amino, (mono- or dialkyl)amino, —CHO, —COOH, —CONH₂, ester, carbamate, urea, sulfonamide, phosphate, phosphonate, and R^{L};
B is selected from halo, nitro, cyano, azido, C₁-C₆alkyl, C₁-C₆haloalkyl, C₂-C₆alkenyl, C₂-C₆alkynyl, C₃-C₇cycloalkyl, (4- to 10-membered monocyclic or bicyclic heterocycle)-(C₀-C₆alkyl), (5- to 10-membered monocyclic or bicyclic aryl)-(C₀-C₆alkyl), (5- to 10-membered monocyclic or bicyclic heteroaryl)-(C₀-C₆alkyl), —OR^{a}, —SR^{a}, —NR^{a}R^{b}, —C(O)R^{c}, —S(O)R^{c}, —S(O)₂R^{c}, and R^{L}, each of which may be optionally substituted with one or more A groups as allowed by valency; and
R^{L} is a linker conjugated to a PROTAC moiety.

Embodiment 2. The compound of embodiment 1, wherein W is CR⁶.

Embodiment 3. The compound of any one of embodiments 1 or 2, wherein X is NR¹.

Embodiment 4. The compound of any one of embodiments 1-3, wherein Y is O.

Embodiment 5. The compound of any one of embodiments 1-4, wherein Z is C(R¹²)(R¹³).

Embodiment 6. The compound of any one of embodiments 1-5, wherein R⁹ is 5- to 10-membered monocyclic or bicyclic heteroaryl.

Embodiment 7. The compound of any one of embodiments 1-6, wherein n is 1.

Embodiment 8. The compound of any one of embodiments 1-7, wherein m is 0.

Embodiment 9. The compound of any one of embodiments 1-8, wherein the compound is of Formula I-a:

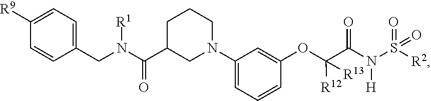

or a pharmaceutically acceptable salt thereof.

Embodiment 10. The compound of any one of embodiments 1-9, wherein the compound is of Formula I-b:

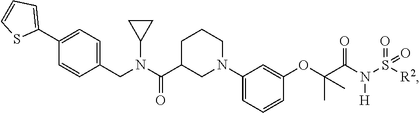

or a pharmaceutically acceptable salt thereof.

Embodiment 11. The compound of any one of embodiments 1-10, wherein R² is C₁-C₆alkyl.

Embodiment 12. The compound of any one of embodiments 1-10, wherein R² is C₃-C₇cycloalkyl.

Embodiment 13. The compound of any one of embodiments 1-10, wherein R² is phenyl optionally substituted with one or more groups selected from hydroxyl, cyano, or C₁-C₆alkoxy.

Embodiment 14. The compound of any one of embodiments 1-13, wherein the linker conjugated to a PROTAC moiety is comprises a linker selected from substituted or unsubstituted C₄-C₂₄alkyl, a substituted or unsubstituted C₄-C₂₄alkoxy, and the PROTAC moiety is selected from a cereblon binder (such as thalidomide, lenalidomide, or pomalidomide) or a von Hippel-Lindau E3 ligase (VHL) ligand (such as VH032 or VH298).

Embodiment 15. The compound of embodiment 1, wherein the compound is selected from the group consisting of:

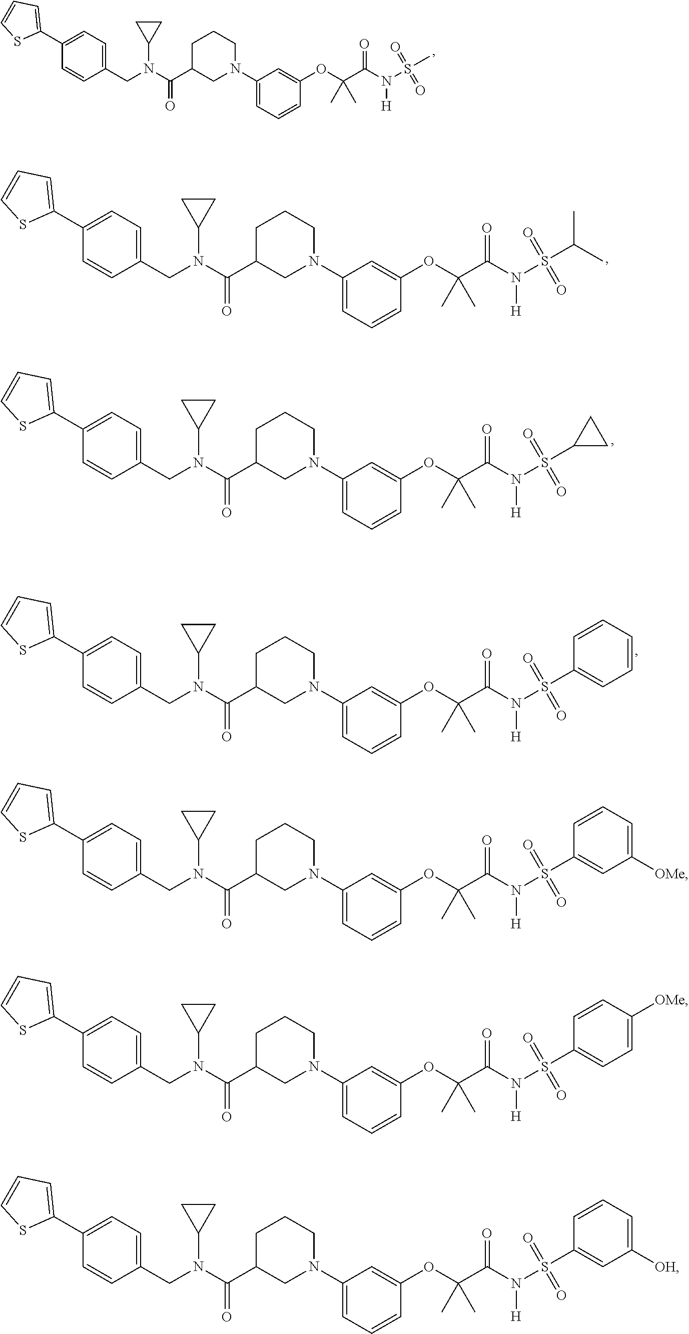

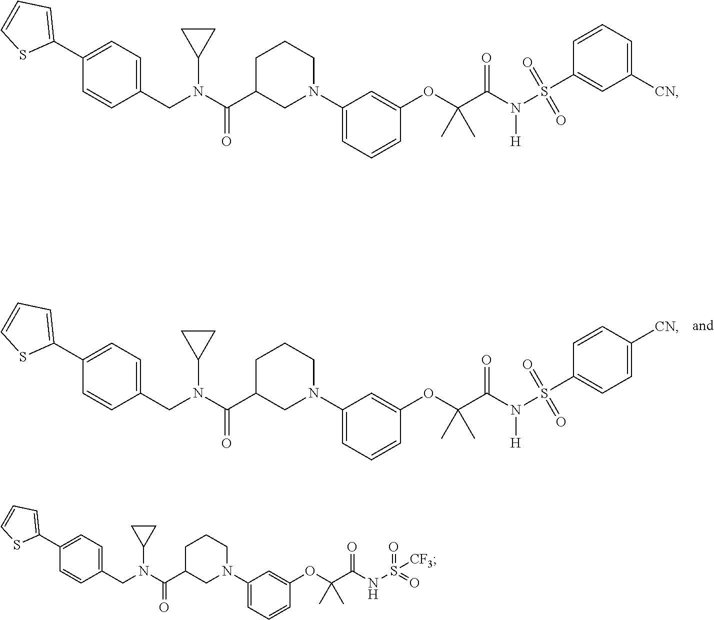 [SMILES: CC(C)(Oc1cccc(N2CCCC(C(=O)N(Cc3ccc(-c4cccs4)cc3)C3CC3)C2)c1)C(=O)NS(=O)(=O)c1cccc(C#N)c1 ; CC(C)(Oc1cccc(N2CCCC(C(=O)N(Cc3ccc(-c4cccs4)cc3)C3CC3)C2)c1)C(=O)NS(=O)(=O)c1ccc(C#N)cc1 ; CC(C)(Oc1cccc(N2CCCC(C(=O)N(Cc3ccc(-c4cccs4)cc3)C3CC3)C2)c1)C(=O)NS(=O)(=O)C(F)(F)F]

or a pharmaceutically acceptable salt thereof.

Embodiment 16. The compound of embodiment 1, wherein the compound is selected from the group consisting of:

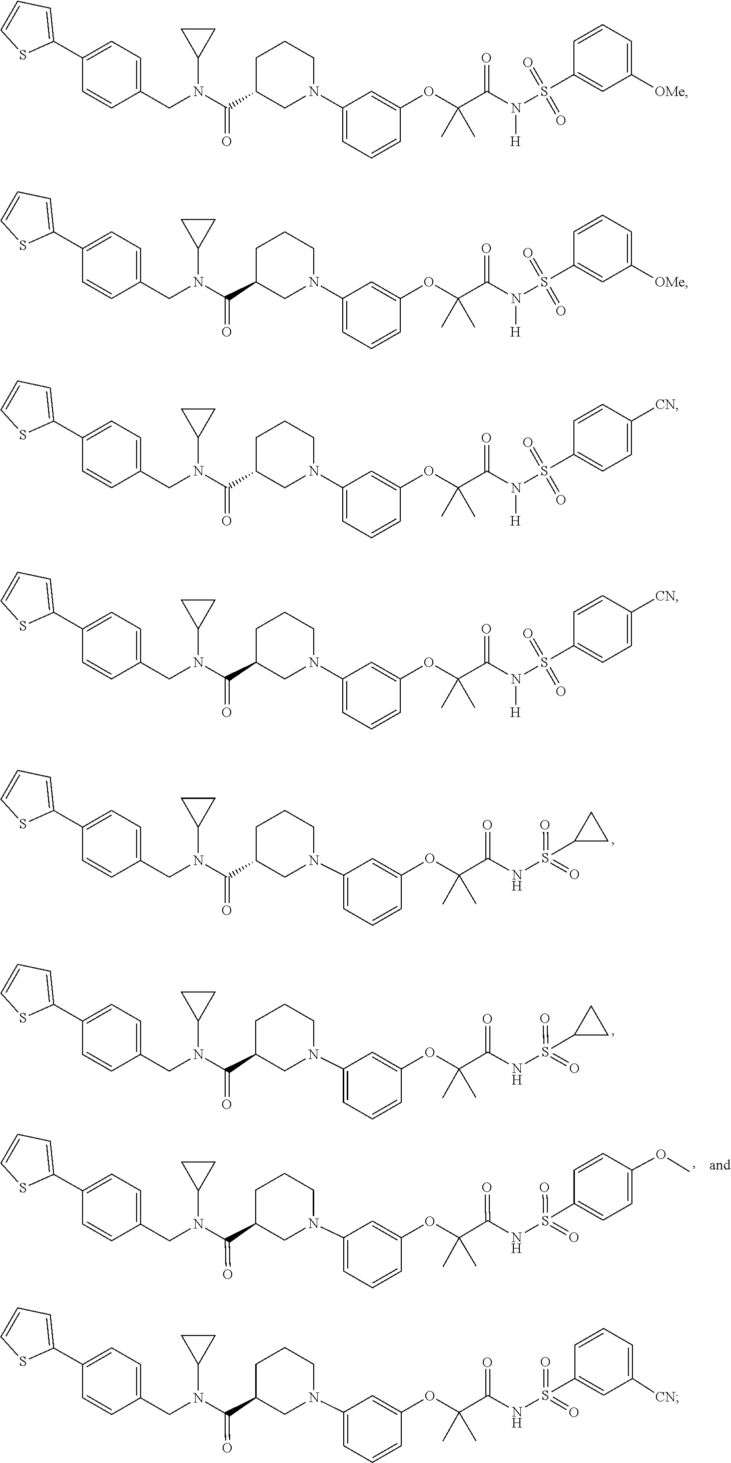

or a pharmaceutically acceptable salt thereof.

Embodiment 17. A pharmaceutical composition comprising a compound of any one of embodiments 1-16, or pharmaceutically acceptable salt thereof, and a pharmaceutically acceptable carrier.

Embodiment 18. A method of treating a disorder of uncontrolled cellular proliferation in a subject comprising administering to the subject a therapeutically effective amount of a compound of any one of embodiments 1-16, or a pharmaceutically acceptable salt thereof.

Embodiment 19. The method of embodiment 18, wherein the compound inhibits β-catenin/B-cell lymphoma 9 complex.

Embodiment 20. The method of any one of embodiments 18 or 19, wherein the compound decreases cellular β-catenin levels.

Embodiment 21. The method of any one of embodiments 18-20, wherein the subject is a human.

Embodiment 22. The method of any one of embodiments 18-21, wherein the disorder is cancer.

Embodiment 23. The method of embodiment 22, wherein the cancer is selected from breast cancer, cervical cancer, gastrointestinal cancer, colorectal cancer, brain cancer, skin cancer, prostate cancer, ovarian cancer, thyroid cancer, testicular cancer, pancreatic cancer, endometrial cancer, melanoma, glioma, leukemia, lymphoma, chronic myeloproliferative disorder, myelodysplastic syndrome, myeloproliferative neoplasm, and plasma cell neoplasm (myeloma).

Embodiment 24. A method for inhibiting protein-protein interactions of β-catenin and B-cell lymphoma 9 in at least one cell comprising contacting the at least one cell with an effective amount of a compound of any one of embodiments 1-16, or a pharmaceutically acceptable salt thereof.

Embodiment 25. A method for degrading β-catenin in at least one cell comprising contacting the at least one cell with an effective amount of a compound of any one of embodiments 1-16, or a pharmaceutically acceptable salt thereof, wherein the compound comprises at least one linker conjugated to a PROTAC moiety.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### Examples

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention, which are apparent to one skilled in the art.

Efforts have been made to ensure accuracy with respect to numbers (e.g., amounts, temperature, etc.), but some errors and deviations should be accounted for. Unless indicated otherwise, parts are parts by weight, temperature is in degrees Celsius or is at ambient temperature, and pressure is at or near atmospheric. There are numerous variations and combinations of reaction conditions, e.g., component concentrations, temperatures, pressures, and other reaction ranges and conditions that can be used to optimize the product purity and yield obtained from the described process. Only reasonable and routine experimentation will be required to optimize such process conditions.

### Example 1. Small-Molecule Inhibitors of the β-Catenin/B-Cell Lymphoma 9 Protein-Protein Interaction

The β-catenin/BCL9 protein-protein interaction (PPI) has emerged as a potential therapeutic target to suppress the hyperactive Wnt/β-catenin signaling that is vigorously involved in tumor initiation and development. Herein, we report the medicinal chemistry optimization on a screening hit to yield a series of novel small-molecule inhibitors of β-catenin/BCL9 PPI. The best compound 30 was found to disrupt the β-catenin/BCL9 PPI with a Kᵢ of 3.6 μM in AlphaScreen competitive inhibition assays. Cell-based studies revealed that 30 selectively disrupted the β-catenin/BCL9 interaction while leaving the β-catenin/E-cadherin complex unaffected, dose-dependently suppressed transactivation of Wnt/β-catenin signaling, downregulated expression of Wnt target genes, and selectively inhibited viability of cancer cells with hyperactive β-catenin signaling. This compound could serve as a lead compound for further optimization of inhibitors of β-catenin/BCL9 PPI.

### Results

Inhibitor design and optimization. Our medicinal chemistry program started with a hit compound, CP-868388, discovered by compound screen using the AlphaScreen competitive inhibition assay.⁴¹ CP868388 after resynthesis inhibited the β-catenin/BCL9 PPI with a Kᵢ of 11 μM in the AlphaScreen assay. Given the carbamate group in 1 is liable to metabolic hydrolysis,⁴² and the chiral carbon is difficult to be constructed,⁴³ the scaffold hopping strategy was adopted to obtain a new scaffold.

Although CP868388 inhibited β-catenin/BCL9 PPI with a Kᵢ of 11 μM in the AlphaScreen assay, it was inactive in cell-based experiments.⁴¹ Careful examination on the chemical structure of CP868388 suggested that the inferior cellular activities might be caused by the carboxylic acid moiety, which was known to negatively impact cell membrane permeability. Therefore, further optimization was conducted on this carboxylic acid group to improve both biochemical and cellular activities. Compounds 16 and 17 featured with methyl ester and tert-butyl ester, respectively, were first synthesized (Table 1), and both compounds showed significant potency loss. This result indicates the negative-charge character of the carboxylic acid group is important for maintaining the inhibitory activity. Based on this observation, a bioisosteric replacement strategy was adopted, and a series of acyl sulfonamide derivatives were designed and synthesized (Table 1). N-Acylsulfonamide is a bioisostere of the carboxylic acid group that is often used to obtain the derivatives with improved pharmacokinetic and pharmacodynamic profiles.⁴⁴ The first designed compound 18 with a N-methylsulfonamide group inhibited β-catenin/BCL9 PPI with a Kᵢ of 21 μM, and thus it is two-fold less potent than 12. Encouragingly, the substitution of the methyl group in N-methylsulfonamide moiety with larger substituents (such as cyclopropyl (19), isopropyl (20), and phenyl(21)) resulted in compounds with improved activities. For instance, compound 21 with N-phenylsulfonamide displayed a Kᵢ value of 6.4 μM, and it is two-fold more potent than 12. To further improve the physicochemical properties of this series of inhibitors, hydrophilic substituents including methoxy, hydroxy, and cyano moieties were introduced to the phenyl group of N-phenylsulfonamide. As shown in Table 1, all these compounds showed increased activities when compared with 21. Specifically, 22 and 26 were two most potent inhibitors with Kᵢ values of 4.1 and 3.9 μM, respectively. Finally, the R- and S-isomers for 22 and 26 were synthesized and their preliminary activities were determined in AlphaScreen assays. The results in Table 2 shows for both compounds the S-isomer (28 and 30) is slightly superior to R-isomer (27 and 29).

TABLE 1
AlphaScreen Assay Results of Inhibitors 16-26

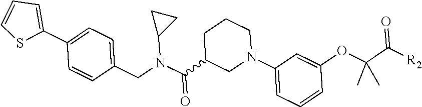

| | | IC₅₀ | Kᵢ |
|---|---|---|---|
| Compounds | R₂ | (μM) | (μM) |
| 16 | OMe | >100 | >90 |
| 17 | O^{t}Bu | >100 | >90 |
| 18 | 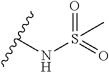 [SMILES: *NS(C)(=O)=O] | ≤30 | ≤30 |
| 19 | 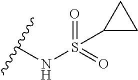 [SMILES: *NS(=O)(=O)C1CC1] | ≤30 | ≤30 |
| 20 | 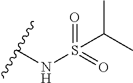 [SMILES: *NS(=O)(=O)C(C)C] | ≤30 | ≤30 |
| 21 | 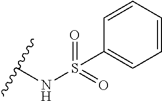 [SMILES: *NS(=O)(=O)c1ccccc1] | ≤10 | ≤10 |
| 22 | 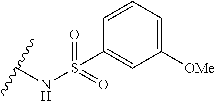 [SMILES: *NS(=O)(=O)c1cccc(OC)c1] | ≤10 | ≤5 |
| 23 | 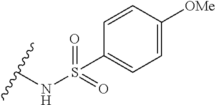 [SMILES: *NS(=O)(=O)c1ccc(OC)cc1] | ≤10 | ≤5 |
| 24 | 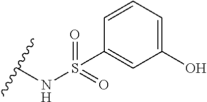 [SMILES: *NS(=O)(=O)c1cccc(O)c1] | ≤10 | ≤10 |
| 25 | 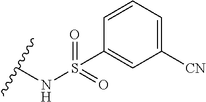 [SMILES: *NS(=O)(=O)c1cccc(C#N)c1] | ≤10 | ≤10 |
| 26 | 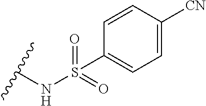 [SMILES: *NS(=O)(=O)c1ccc(C#N)cc1] | ≤5 | ≤5 |

TABLE 2
AlphaScreen Assay Results of Inhibitors 27-30

| | | IC₅₀ | Kᵢ |
|---|---|---|---|
| Cmpds | Structure | (μM) | (μM) |
| 27 | 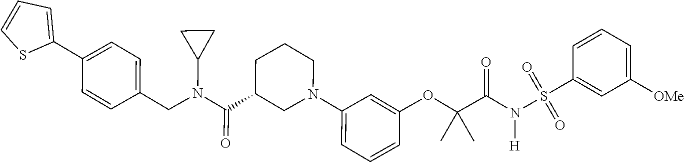 [SMILES: COc1cccc(S(=O)(=O)NC(=O)C(C)(C)Oc2cccc(N3CCC[C@@H](C(=O)N(Cc4ccc(-c5cccs5)cc4)C4CC4)C3)c2)c1] | ≤10 | ≤5 |
| 28 | 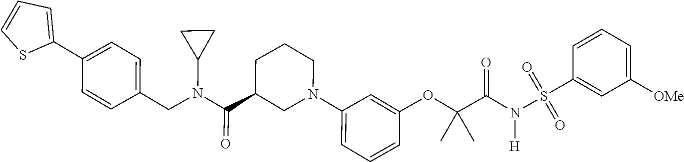 [SMILES: COc1cccc(S(=O)(=O)NC(=O)C(C)(C)Oc2cccc(N3CCC[C@H](C(=O)N(Cc4ccc(-c5cccs5)cc4)C4CC4)C3)c2)c1] | ≤5 | ≤5 |
| 29 | 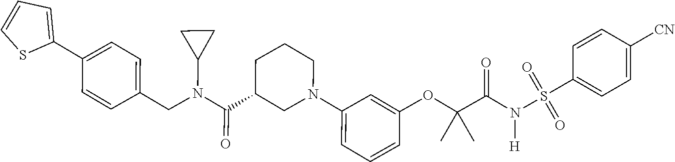 [SMILES: CC(C)(Oc1cccc(N2CCC[C@@H](C(=O)N(Cc3ccc(-c4cccs4)cc3)C3CC3)C2)c1)C(=O)NS(=O)(=O)c1ccc(C#N)cc1] | ≤5 | ≤5 |
| 30 | 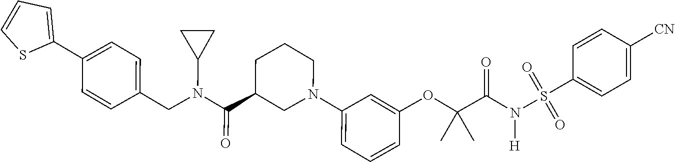 [SMILES: CC(C)(Oc1cccc(N2CCC[C@H](C(=O)N(Cc3ccc(-c4cccs4)cc3)C3CC3)C2)c1)C(=O)NS(=O)(=O)c1ccc(C#N)cc1] | ≤5 | ≤5 |

TABLE 3
AlphaScreen Assay Results of Inhibitors 31-35

| | | IC₅₀ | Kᵢ |
|---|---|---|---|
| Cmpds | Structure | (μM) | (μM) |
| 31 | 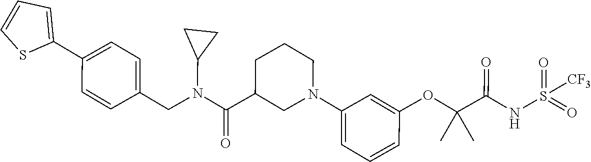 [SMILES: CC(C)(Oc1cccc(N2CCCC(C(=O)N(Cc3ccc(-c4cccs4)cc3)C3CC3)C2)c1)C(=O)NS(=O)(=O)C(F)(F)F] | ~200 | ~168 |
| 32 | 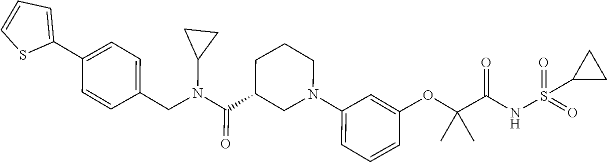 [SMILES: CC(C)(Oc1cccc(N2CCC[C@@H](C(=O)N(Cc3ccc(-c4cccs4)cc3)C3CC3)C2)c1)C(=O)NS(=O)(=O)C1CC1] | ≤30 | ≤30 |
| 33 | 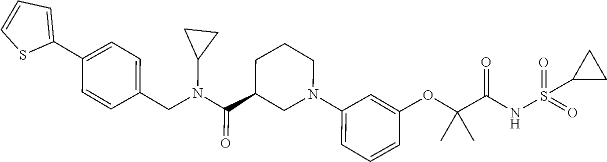 [SMILES: CC(C)(Oc1cccc(N2CCC[C@H](C(=O)N(Cc3ccc(-c4cccs4)cc3)C3CC3)C2)c1)C(=O)NS(=O)(=O)C1CC1] | ≤30 | ≤30 |
| 34 | 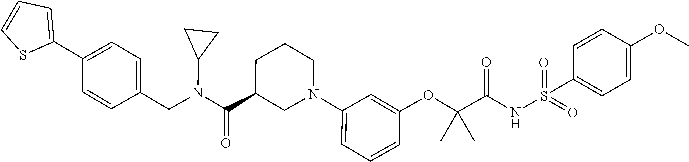 [SMILES: COc1ccc(S(=O)(=O)NC(=O)C(C)(C)Oc2cccc(N3CCC[C@H](C(=O)N(Cc4ccc(-c5cccs5)cc4)C4CC4)C3)c2)cc1] | ≤30 | ≤30 |
| 35 | 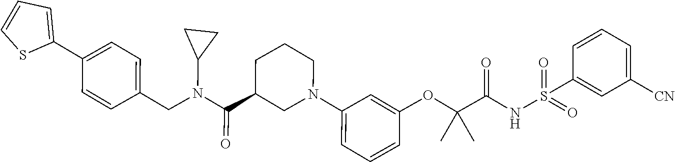 [SMILES: CC(C)(Oc1cccc(N2CCC[C@H](C(=O)N(Cc3ccc(-c4cccs4)cc3)C3CC3)C2)c1)C(=O)NS(=O)(=O)c1cccc(C#N)c1] | ≤10 | ≤10 |

Cell-based studies. MTS cell viability assays were performed to evaluate the effects of β-catenin/BCL9 inhibitors on growth of different cancer cell lines with hyperactive Wnt/β-catenin signaling, including colorectal cancer cells (SW480 and HCT116) and triple-negative breast cancer cells (MDA-MB-231 and MDA-MB-468). The normal breast epithelial MCF10A cells were also assessed in parallel for comparison, and the results are shown in Table 3. The representative compound 30 inhibited viability of Wnt/β-catenin hyperactive cancer cells with IC₅₀s between 14 and 30 μM, while sparing normal mammary epithelial MCF10A cells with 5- to 10-fold selectivity. Although compound 28 displayed similar IC₅₀s for Wnt/β-catenin hyperactive cancer cells, it was much less selective over MCF10A cells. In addition, an inactive analogue 17 in AlphaScreen assay (Kᵢ>90 μM, Table 1) did not show any inhibitory effects (IC₅₀>200 μM, Table 3) on all tested cells.

TABLE 3
The MTS Assay to Monitor the Inhibitory Activities
of 17, 28, and 30 on Cell Viability^{a}
MTS IC₅₀ ± SD (μM)

| | | | | | Wnt/β-catenin |
|---|---|---|---|---|---|
| | Wnt/β-catenin hyperactive | | | | normal |
| No. | SW480 | HCT116 | MDA-MB-231 | MDA-MB-468 | MCF10A |
| 17 | >200 | >200 | >200 | >200 | >200 |
| 28 | 28.2 ± 1.08 | 29.9 ± 1.99 | 19.8 ± 1.59 | 15.4 ± 2.49 | 30.4 ± 2.47 |
| 30 | 16.3 ± 2.04 | 29.6 ± 1.02 | 14.3 ± 0.570 | 17.8 ± 1.01 | 139 ± 3.16 |

^{a}Each set of data was expressed as mean ± standard deviation (n = 3).

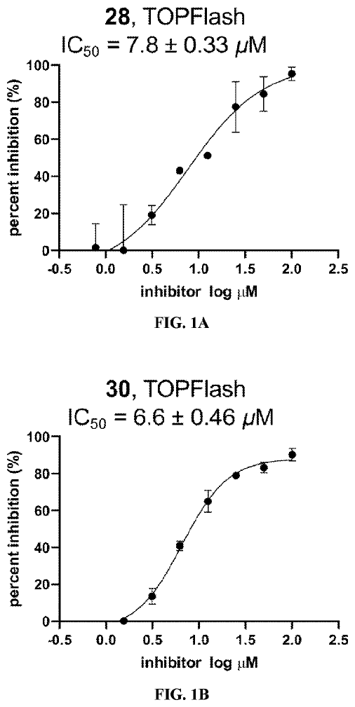

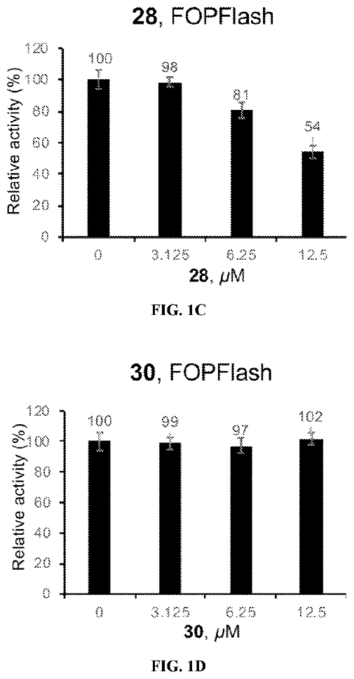

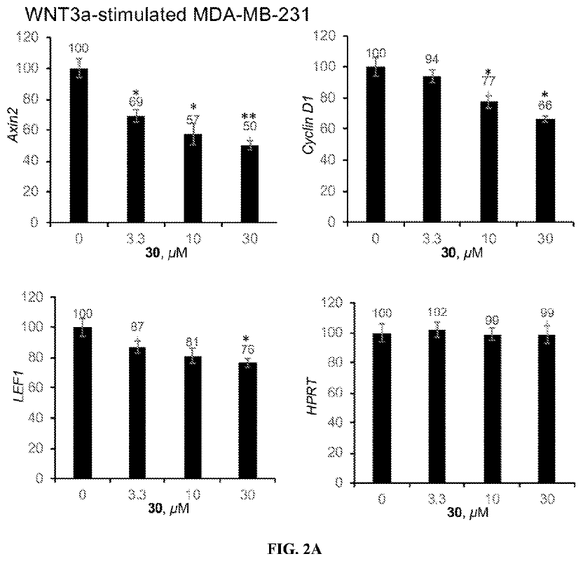

The suppressing effect of the inhibitors on transactivation of Wnt/β-catenin signaling was evaluated by the TOPFlash/FOPFlash luciferase reporter assay. In the TOPFlash assay, the luciferase reporter has three wild-type Tcf4 binding sites, while in the FOPFlash assay Tcf4 binding sites were mutated. These tandem Tcf binding sites controls the expression ratio of the firefly luciferase in TOPFlash/FOPFlash assays. The renilla luciferase reporter construct (pCMV-RL) was used as the internal control to normalize luciferase reporter signals and eliminate any possible systematic errors. As shown in FIGS. 1A-1D, both compounds 28 and 30 dose-dependently inhibited the TOPFlash luciferase activity with the IC₅₀ values of 7.8 and 6.6 μM, respectively. However, at this concentration range, only 30 did not show any inhibitory effect on the FOPFlash luciferase reporter activity, indicating that 30 is specific for the Wnt/β-catenin signaling pathway. This selectivity trend is consistent with that from MTS cell viability assays. Therefore, further cell-based studies were focused on compound 30.

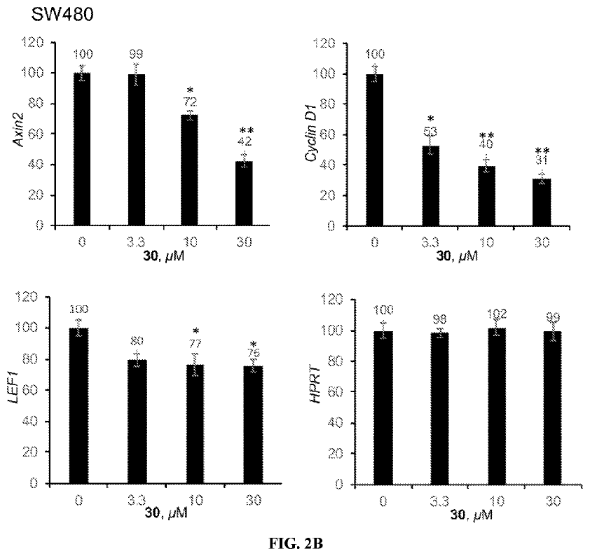

Quantitative real-time PCR (qPCR) studies were conducted to evaluate the effects of 30 on transcription of Wnt-specific target gene Axin2 and two other Wnt target genes, cyclin D1 and LEF1. The results showed that 30 dose-dependently inhibited transcription of these Wnt target genes while sparing the house-keeper gene HPRT in both Wnt 3a-activated MDA-MB-231 and SW480 cells (FIGS. 2A and 2B).

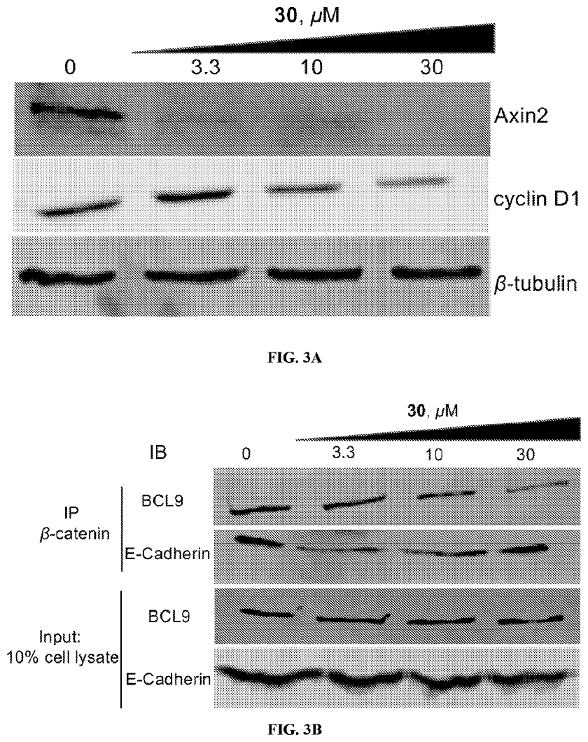

Western blot experiments were performed to determine the protein expression levels of Axin2 and cyclin D1. As shown in FIG. 3A, treatment of compound 30 dose-dependently decreased the protein expression levels of both Axin2 and cyclin D1.

C₀-immunoprecipitation (co-IP) experiments were conducted to assess effect of 30 on disruption of the interaction between full-length β-catenin and full-length BCL9 in the cellular context. As shown in FIG. 3B, compound 30 disrupted the β-catenin/BCL9 PPI in a dose-dependent manner. A parallel experiment showed that 30 did not affect the β-catenin/E-cadherin PPI at the concentrations that were sufficient to disrupt the β-catenin/BCL9 PPI. This is important because the surface areas of β-catenin for binding with E-cadherin and BCL9 are overlapped, and β-catenin/E-cadherin complex is critical for cell-cell adhesion.

### Chemistry

The synthetic routes for 12 are shown in Scheme 1. The intermediate 35 was synthesized through the reductive amination reaction using 4-bromobenzaldehyde and cyclopropanamine as the starting materials. Buchwald-Hartwig amination between ethyl piperidine-3-carboxylate and 31 yielded 36. Hydrolysis of the ethyl ester in 36 and then amide coupling with 35 produced key intermediate 37, which underwent Suzuki coupling with different boronic acids to generate 38. Removal of the tert-butyl group in 38 by TFA/CH₂Cl₂ yielded 12.

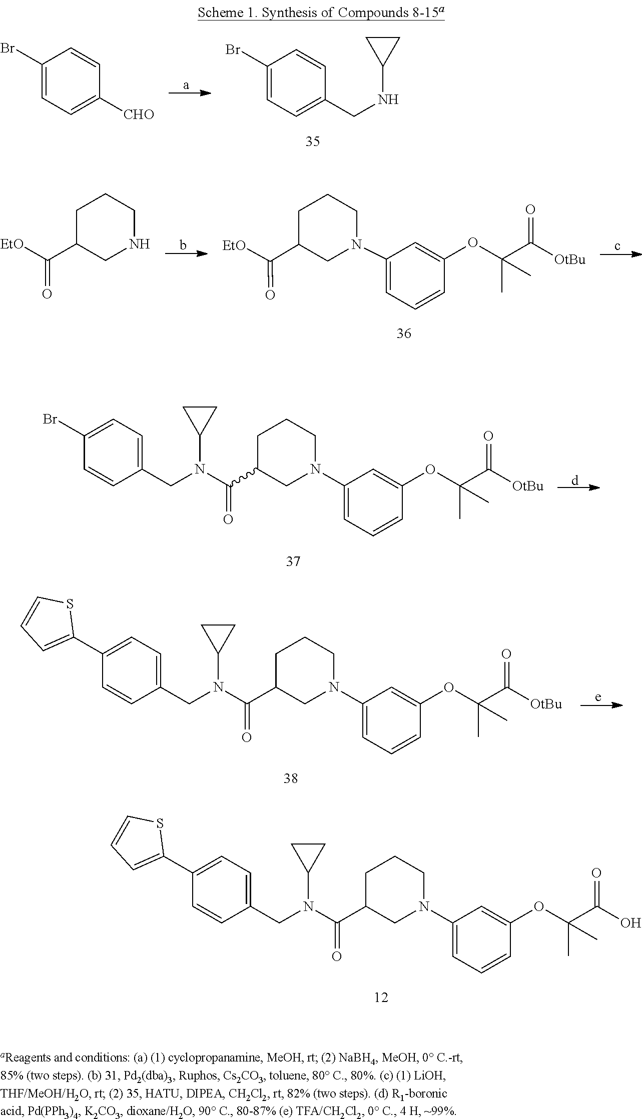

The synthetic routes for final products 16-26 are shown in Scheme 2. Compounds 16 and 17 were obtained by the ester formation reactions between 12 and methanol or tert-butanol, respectively. The other compounds (18-26) were achieved by the reactions between 12 and various sulfonamides.

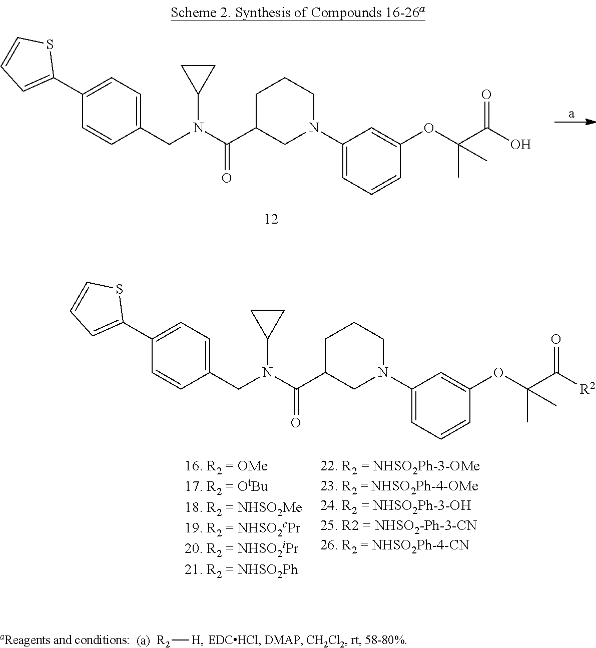

The synthetic routes for final products 27 and 29 are shown in Scheme 3. Ethyl(R)-piperidine-3-carboxylate underwent Buchwald-Hartwig amination with 31 yielded 39. Hydrolysis of the ethyl ester in 39 and then amide coupling with 35 gave intermediate 40. The Suzuki coupling reaction between 40 and thiophen-2-ylboronic acid produced 41. Removing the tert-butyl group in 41 under acidic condition and then reacting with 3-methoxybenzenesulfonamide or 4-cyanobenzenesulfonamide yielded final products 27 and 29, respectively.

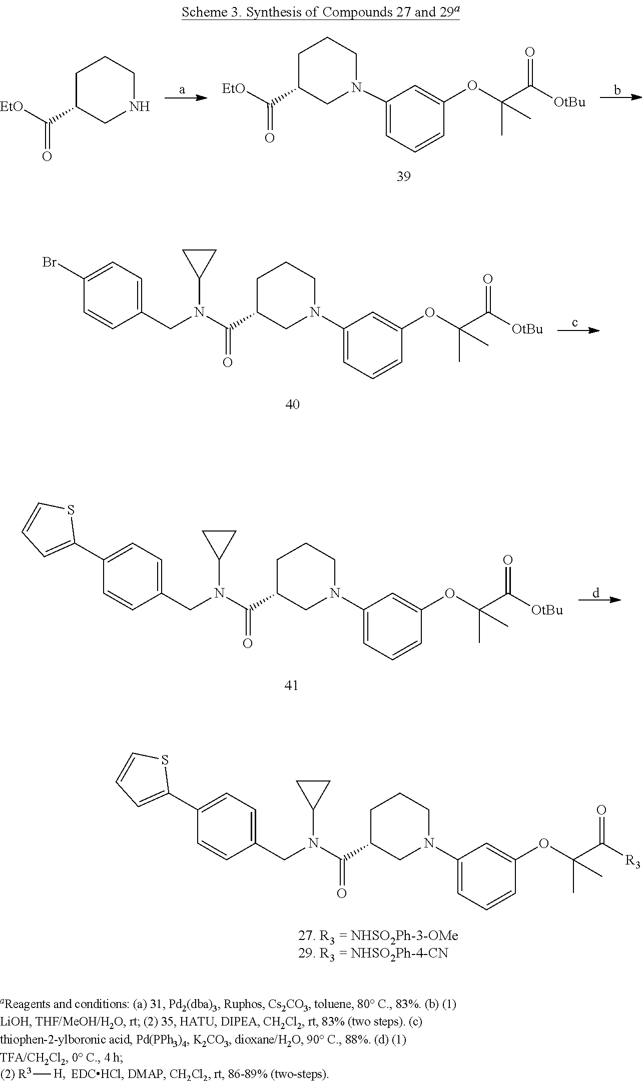

The synthetic routes for final products 28 and 30 are shown in Scheme 4. Ethyl(S)-piperidine-3-carboxylate went through Buchwald-Hartwig amination with 31 to yield 42. Hydrolysis of the ethyl ester in 42 and then amide coupling with 35 gave intermediate 43, which underwent the Suzuki coupling reaction with thiophen-2-ylboronic acid to produce 44. Final products 28 and 30 were obtained by removing the tert-butyl group in 44 and then reacting with 3-methoxybenzenesulfonamide or 4-cyanobenzenesulfonamide, respectively.

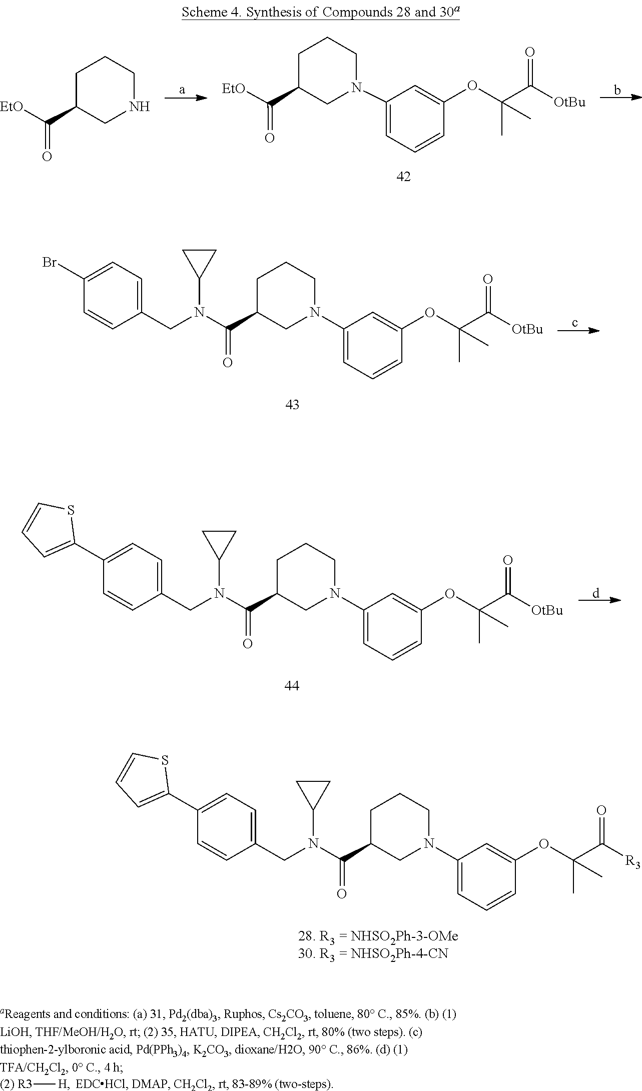

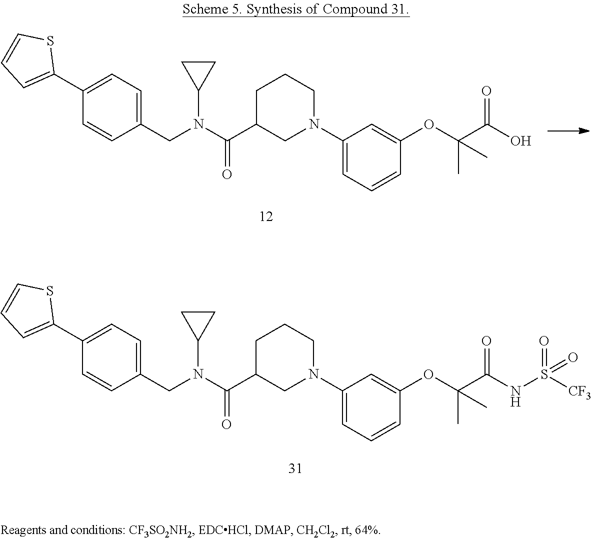 [SMILES: CC(C)(Oc1cccc(N2CCCC(C(=O)N(Cc3ccc(-c4cccs4)cc3)C3CC3)C2)c1)C(=O)O ; CC(C)(Oc1cccc(N2CCCC(C(=O)N(Cc3ccc(-c4cccs4)cc3)C3CC3)C2)c1)C(=O)NS(=O)(=O)C(F)(F)F]

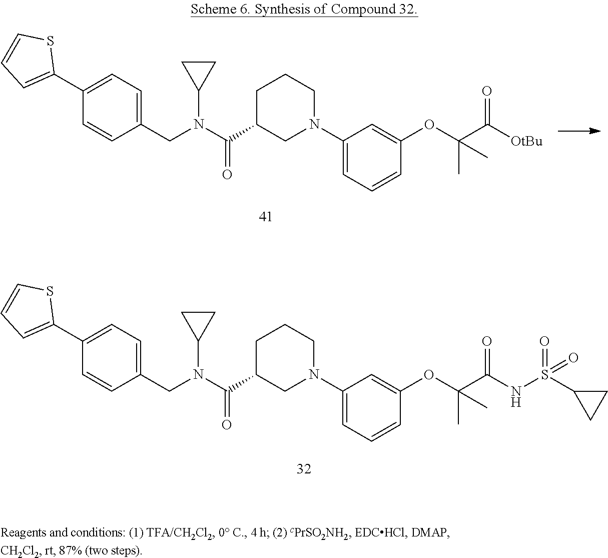 [SMILES: CC(C)(C)OC(=O)C(C)(C)Oc1cccc(N2CCC[C@@H](C(=O)N(Cc3ccc(-c4cccs4)cc3)C3CC3)C2)c1 ; CC(C)(Oc1cccc(N2CCC[C@@H](C(=O)N(Cc3ccc(-c4cccs4)cc3)C3CC3)C2)c1)C(=O)NS(=O)(=O)C1CC1]

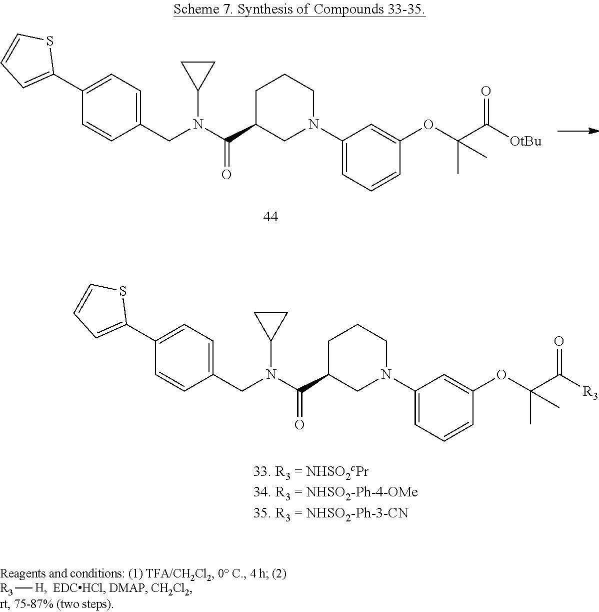

### Discussion and Conclusions

The Wnt/β-catenin signaling pathway is frequently overactivated in many cancers. Targeting the downstream β-catenin-containing transcriptional complex, rather than the upstream effectors, appears to the most effective approach for suppression of the hyperactive Wnt/β-catenin signaling. However, disruption of β-catenin-related PPI in the transcriptional complex has proven to be a formidable challenge. Most β-catenin-directed programs were aimed at β-catenin/Tcf PPI, but very limited progress has been made. On one hand, the contacting area between β-catenin and Tcf is very large (3500 Å²) and the binding affinity is high (K_{D}=7-10 nM), suggesting that this PPI might be too difficult to be disrupted by small molecules.⁴⁵ On the other hand, β-catenin uses the same surface to interact with Tcf, APC, and E-cadherin, indicating it would be challenging to achieve high inhibitor selectivity.⁴⁵ On the contrary, in the β-catenin-containing transcriptional complex BCL9 binds with β-catenin via a single α-helical structure (the HD2 domain).⁴⁵ β-Catenin and BCL9 have much smaller contacting interface areas (1450 Å²) and their binding affinity is moderate (K_{D}=0.47 μM). The only reported additional binding partner at this PPI interface is E-cadherin. Therefore, the β-catenin/BCL9 complex emerges as a promising alternative target to suppress the overactivated Wnt/β-catenin signaling. In this work, structural optimization based on a screening hit 1 successfully yielded a series of novel selective β-catenin/BCL9 inhibitors. Cell-based co-IP experiments shows the representative compound 30 not only disrupts-catenin/BCL9 PPI, but also exhibits selectivity for β-catenin/BCL9 over β-catenin/E-cadherin PPIs.

This small-molecule β-catenin/BCL9 disruptor 30 we identified is structurally different from carnosic acid and PNPB series, the reported β-catenin/BCL9 inhibitors and represents a new direction for future inhibitor optimization. It is worth noting both this series of inhibitors and carnosic acid contain an acid functional group. The natural product scaffold of carnosic acid makes it difficult for further inhibitor optimization. Further, the catechol group of carnosic acid is potentially liable for oxidization and subsequent reactions with protein nucleophiles, and has been identified as a pan-assay interference compounds (PAINS) moiety. Compound PNPB-22 contains multiple aromatic rings and two positively charged centers. This lipophilic base character makes PNPB-22 toxic to normal cells at high concentrations. PNPB-29 has two positively charged centers and one negatively charged center. This zwitterion has a low cellular activity. Compound 30 has a novel scaffold and is ready for further optimization. As a result, 30 not only inhibited viability of Wnt/β-catenin hyperactive cancer cells, but also displayed selectivity toward normal breast epithelial MCF10A cells in MTS cell viability assays.

In summary, the β-catenin/BCL9 PPI emerged as an appealing therapeutic target to suppress the overactivated β-catenin signaling for targeted cancer therapy. Our medicinal chemistry studies on a screening hit successfully yielded a series of novel small-molecule inhibitors for β-catenin/BCL9 PPI. The representative compound 30 was found to disrupt the β-catenin/BCL9 PPI with the Kᵢ of 3.6 μM in the AlphaScreen assay. It is noted that although the initial hit 1 displayed a Kᵢ of 11 μM for disruption of the β-catenin/BCL9 PPI in the AlphaScreen assay, it did not show any cellular activity. Encouragingly, cell-based studies revealed the obtained new inhibitor 30 disrupted the β-catenin/BCL9 interaction while sparing the β-catenin/E-cadherin complex, dose-dependently suppressed transactivation of Wnt/β-catenin signaling, downregulated expression of Wnt target genes, and selectively inhibited viability of Wnt/β-catenin-dependent cancer cells. This new inhibitor can serve as a lead compound for further optimization of inhibitors for β-catenin/BCL9 PPI.

### Experimental Section

Chemical Synthesis. General Methods, Reagents, and Materials. All reagents were purchased from commercial sources (Combi-Blocks, Inc.; Oakwood Products, Inc.; Fisher Scientific; and VWR International, LLC) and used without further purification. 1H NMR and ¹³C NMR spectra were recorded on Bruker AVANCEIIIHD 500 (500 MHZ) spectrometers (125.7 MHz for ¹³C NMR spectra) in d₆-DMSO, d₄-methanol, and CDCl₃. Chemical shifts were reported as values in parts per million (ppm), and the reference resonance peaks were set at 7.26 ppm (CHCl₃), 3.31 ppm (CD₂HOD), and 2.50 ppm [(CD₂H)₂SO] for ¹H NMR spectra and 77.23 ppm (CDCl₃), 49.00 ppm (CD₃OD), and 39.52 ppm (d₆-DMSO) for ¹³C NMR spectra. Low-resolution mass spectra were determined on Agilent 6120 single quadrupole MS with 1220 infinity LC system (HPLC-MS) and an ESI source. High-resolution mass spectra were determined on Agilent G6230BA TOF LCMS Mass Spectrometer with a TOF mass detector. Thin-layer chromatography was carried out on E. Merck pre-coated silica gel 60 F254 plates with a UV-visible lamp. Column chromatography was performed with SilicaFlash@ F60 (230-400 mesh). The purity of final compounds 2-30 was determined by HPLC analyses. The instrument was an Agilent 1260 Infinity II HPLC system with a quaternary pump, a vial sampler, and a DAD detector. A Phenomenex C18 column (Luna 5 μL C18 (2) 100 Å, 4.6×250 mm) was used. The DAD detector was set to 254 nm. The purity of all tested compounds was >95%.

General procedure for reductive-amination reaction. A solution of aldehyde (1 mmol) and amine (2 mmol) in anhydrous methanol (5 mL) was stirred under Ar at room temperature overnight. Then, NaBH₄ (1.5 mmol) was added portion-wise to the mixture at 0° C. The mixture was stirred at 0° C. for 1 h. Saturated NH₄Cl was added, followed by addition of ethyl acetate. The organic phase was collected and washed with brine, dried over anhydrous Na₂SO₄, concentrated under reduced pressure, and purified by column chromatography to yield the target compound.

General procedure for amide coupling reaction. At 0° C., to a suspension of carboxylic acid (1 mmol), amine (1.1 mmol), HATU (1.5 mmol) in dichloromethane (CH₂Cl₂) (10 mL) was added N, N-diisopropylethylamine (DIPEA) (2 mmol) dropwise. The reaction mixture was warmed to room temperature and stirred overnight. At the completion of the reaction, more CH₂Cl₂ was added, and the organic phase was washed by 1 M HCl, saturated NaHCO₃, brine, dried over anhydrous Na₂SO₄, and concentrated under reduced pressure. Column chromatography was used to purify the target compound.

General procedure for deprotection of Boc using 50% trifluoroacetic acid (TFA) in CH₂Cl₂. To Boc-protected amine (1 mmol) in 3 mL CH₂Cl₂ at room temperature was added TFA (3 mL). The reaction was stirred at room temperature for 3 h. TFA was removed by adding CH₂Cl₂ three times to afford the desired product, which was used directly in next step. General procedure for Buchwald-Hartwig amination reaction. A solution of bromobenzene derivatives (1 mmol), amine (1.1 mmol), Pd₂ (dba) 3 (0.1 mmol), RuPhos (0.2 mmol), and Cs₂CO₃ (2 mmol) in toluene (10 mL) was heated to 80° C. under argon and stirred overnight. The reaction mixture was cooled to room temperature. The solid was removed, and the filtrate was concentrated under reduced pressure and purified by column chromatography to yield the target compound.

General procedure for deprotection of tert-butyl using 50% TFA in CH₂Cl₂. At 0° C., to a solution of tert-butyl ester (1 mmol) in CH₂Cl₂ (5 mL) was added TFA (5 mL) dropwise. The reaction was stirred at 0° C. for 6 h. Upon completion, the solvent was removed under the reduced pressure. TFA was removed by adding CH₂Cl₂ three times to afford the desired product, which was further purified by column chromatography.

General procedure for hydrolysis of ethyl ester. To a solution of ethyl ester (1 mmol) in THF (4 mL) and methanol (2 mL) was added a solution of LiOH (2 mmol) in H₂O (1 mL). The mixture was stirred at room temperature for 3 h. At the completion of the reaction, the solvent was removed under the reduced pressure, and the residue was re-dissolved in H₂O and was acidified using 1 N HCl. Ethyl acetate was added and the organic phase was washed by brine, dried over anhydrous Na₂SO₄, and concentrated under reduced pressure to yield target compound, which was used directly in next step.

General procedure for Suzuki coupling reaction. To a solution of bromobenzene derivatives (1 mmol) in dioxane/water (12 mL/4 mL) was added boronic acid (1.2 mmol), Pd(PPh₃)₄ (0.1 mmol), and K₂CO₃ (2 mmol). The reaction mixture was heated to 90° C. under argon and stirred overnight. The reaction mixture was cooled to room temperature, concentrated under reduced pressure, then re-dissolved with ethyl acetate, washed with water and brine, and dried over Na₂SO₄. The solution obtained was concentrated under vacuum. The residue was purified by column chromatography to yield the target compound.

General procedure for coupling reaction between carboxylic acid and alcohol or sulfonamide. To a solution of carboxylic acid (1 mmol) and alcohol (1 mmol) or sulfonamide (1 mmol) in CH₂Cl₂ was added EDC·HCl (1.5 mmol) and DMAP (2 mmol). The mixture was stirred at room temperature overnight. More CH₂Cl₂ was added to the mixture and the organic phase was washed by IN HCl and concentrated under reduced pressure. The pure compound was obtained by column chromatography.

Synthesis of tert-butyl 2-(3-bromophenoxy)-2-methylpropanoate. A solution of 3-bromophenol (1 mmol), tert-butyl 2-bromo-2-methylpropanoate (5 mmol), K₂CO₃ (4 mmol), and MgSO₄ (1 mmol) in DMF was stirred at 100° C. overnight. The DMF was removed under reduced pressure. Water and ethyl acetate were added. The obtained organic phase was washed with brine, dried over Na₂SO₄, and concentrated under vacuum. The residue was purified by column chromatography to yield the target compound.

N-(4-Bromobenzyl)cyclopropanamine (35). ¹H NMR (500 MHZ, Chloroform-d) δ 7.46-7.37 (m, 2H), 7.22-7.08 (m, 2H), 3.79 (s, 2H), 2.12 (tt, J=6.5, 3.6 Hz, 1H), 0.49-0.39 (m, 2H), 0.39-0.25 (m, 2H). MS (ESI) m/z=226.0, 228.0 [M+H]⁺.

Ethyl 1-(3-((1-(tert-butoxy)-2-methyl-1-oxopropan-2-yl)oxy)phenyl) piperidine-3-carboxylate (36). 1H NMR (500 MHz, Chloroform-d) δ 7.04 (t, J=8.2 Hz, 1H), 6.54 (dd, J=8.1, 2.3 Hz, 1H), 6.45 (t, J=2.4 Hz, 1H), 6.31-6.21 (m, 1H), 4.12 (q, J=7.1 Hz, 2H), 3.67 (ddt, J=12.4, 3.5, 1.5 Hz, 1H), 3.42 (ddd, J=12.3, 4.9, 3.1 Hz, 1H), 2.95 (dd, J=12.4, 9.9 Hz, 1H), 2.80-2.68 (m, 1H), 2.60 (tt, J=10.0, 3.9 Hz, 1H), 2.05-1.93 (m, 1H), 1.74 (th, J=9.2, 3.1 Hz, 1H), 1.69-1.59 (m, 2H), 1.53 (s, 6H), 1.41 (s, 9H), 1.24 (t, J=7.2 Hz, 3H). MS (ESI) m/z=392.3 [M+H]⁺, 414.3 [M+Na]⁺.

tert-Butyl 2-(3-(3-((4-bromobenzyl) (cyclopropyl) carbamoyl) piperidin-1-yl)phenoxy)-2-methylpropanoate (37). 1H NMR (500 MHZ, Chloroform-d) δ 7.42 (d, J=8.3 Hz, 2H), 7.11 (d, J=8.3 Hz, 2H), 7.07 (t, J=8.2 Hz, 1H), 6.54 (d, J=8.2 Hz, 1H), 6.48 (s, 1H), 6.30 (d, J=8.1 Hz, 1H), 4.59 (d, J=14.7 Hz, 1H), 4.49 (d, J=14.7 Hz, 1H), 3.67 (d, J=12.1 Hz, 2H), 3.42 (s, 1H), 2.97 (t, J=11.6 Hz, 1H), 2.76 (t, J=11.0 Hz, 1H), 2.58 (s, 1H), 1.91 (d, J=8.5 Hz, 1H), 1.76 (dt, J=32.5, 8.6 Hz, 3H), 1.55 (d, J=1.9 Hz, 6H), 1.43 (d, J=2.0 Hz, 9H), 0.91-0.74 (m, 4H). MS (ESI) m/z=571.2, 573.2 [M+H]⁺.

tert-Butyl 2-(3-(3-(cyclopropyl(4-(thiophen-2-yl)benzyl)carbamoyl)piperidin-1-yl)phenoxy)-2-methylpropanoate (38e). 1H NMR (500 MHZ, Chloroform-d) δ 7.50-7.43 (m, 2H), 7.19 (dd, J=3.6, 1.2 Hz, 1H), 7.18 (s, 3H), 7.02-6.92 (m, 2H), 6.47 (dd, J=8.3, 2.3 Hz, 1H), 6.41 (t, J=2.4 Hz, 1H), 6.22 (dd, J=8.1, 2.2 Hz, 1H), 4.58 (d, J=14.7 Hz, 1H), 4.47 (d, J=14.7 Hz, 1H), 3.66-3.55 (m, 2H), 3.36 (tt, J=10.9, 3.4 Hz, 1H), 2.92 (dd, J=12.5, 10.9 Hz, 1H), 2.68 (td, J=12.1, 2.7 Hz, 1H), 2.52 (tt, J=6.9, 4.1 Hz, 1H), 1.89-1.83 (m, 1H), 1.74-1.59 (m, 3H), 1.46 (s, 6H), 1.34 (s, 9H), 0.83-0.65 (m, 4H). MS (ESI) m/z=575.4 [M+H]⁺.

2-(3-(3-(Cyclopropyl(4-(thiophen-2-yl)benzyl)carbamoyl)piperidin-1-yl)phenoxy)-2-methylpropanoic acid (12). 1H NMR (500 MHZ, Chloroform-d) δ 7.50-7.45 (m, 2H), 7.21 (dd, J=3.6, 1.2 Hz, 1H), 7.19-7.17 (m, 1H), 7.14 (d, J=8.0 Hz, 2H), 7.02-6.97 (m, 2H), 6.56 (dd, J=8.2, 2.3 Hz, 1H), 6.50 (d, J=2.4 Hz, 1H), 6.33 (dd, J=8.1, 2.3 Hz, 1H), 4.64 (d, J=14.7 Hz, 1H), 4.42 (d, J=14.7 Hz, 1H), 3.56 (d, J=11.6 Hz, 2H), 3.41 (tt, J=11.4, 3.6 Hz, 1H), 2.93 (t, J=11.6 Hz, 1H), 2.65 (td, J=11.9, 3.3 Hz, 1H), 2.51 (tt, J=6.9, 4.0 Hz, 1H), 1.85 (d, J=7.8 Hz, 1H), 1.74-1.62 (m, 3H), 1.50 (s, 6H), 0.83-0.71 (m, 4H). ¹³C NMR (126 MHZ, Chloroform-d) δ 177.23, 177.06, 155.80, 152.12, 144.06, 137.48, 133.36, 129.49, 128.27, 128.03, 126.11, 124.74, 123.03, 111.51, 111.17, 109.14, 79.44, 52.36, 50.56, 49.60, 39.68, 29.87, 27.58, 25.27, 25.19, 24.35, 9.56, 8.98. HRMS (ESI) Calcd for C₃₀H₃₄N₂O₄S (M−H)⁻ 517.2167, found 517.2176. HPLC purity 99.2%, t_{R}=13.56 min.

Methyl 2-(3-(3-(cyclopropyl(4-(thiophen-2-yl)benzyl)carbamoyl)piperidin-1-yl)phenoxy)-2-methylpropanoate (16). ¹H NMR (500 MHZ, Chloroform-d) δ 7.58-7.53 (m, 2H), 7.29-7.24 (m, 4H), 7.09-7.04 (m, 2H), 6.64-6.54 (m, 1H), 6.48 (s, 1H), 6.25 (dd, J=8.2, 2.2 Hz, 1H), 4.67 (d, J=14.7 Hz, 1H), 4.57 (d, J=14.8 Hz, 1H), 3.74 (s, 3H), 3.71-3.66 (m, 2H), 3.45 (s, 1H), 3.01 (t, J=11.7 Hz, 1H), 2.82-2.74 (m, 1H), 2.61 (ddt, J=10.8, 6.9, 4.0 Hz, 1H), 2.01-1.93 (m, 1H), 1.77 (ddd, J=20.6, 17.2, 8.3 Hz, 3H), 1.58 (s, 6H), 0.91-0.82 (m, 4H). ¹³C NMR (126 MHZ, Chloroform-d) δ 176.82, 175.03, 156.36, 144.11, 137.69, 133.32, 129.43, 128.28, 128.01, 126.11, 124.71, 123.00, 110.67, 109.34, 107.94, 78.95, 52.47, 52.27, 50.07, 49.50, 39.80, 29.77, 27.78, 25.47, 25.39, 24.42, 9.47, 9.14. HRMS (ESI) Calcd for C31H36N2O4S (M+H)⁺533.2469, found 533.2469. HPLC purity 99.0%, t_{R}=14.44 min.

tert-Butyl 2-(3-(3-(cyclopropyl(4-(thiophen-2-yl)benzyl)carbamoyl)piperidin-1-yl)phenoxy)-2-methylpropanoate (17). 1H NMR (500 MHZ, Chloroform-d) δ 7.53-7.42 (m, 2H), 7.24-7.12 (m, 4H), 7.04-6.93 (m, 2H), 6.48 (dd, J=8.2, 2.3 Hz, 1H), 6.41 (t, J=2.3 Hz, 1H), 6.22 (dd, J=8.1, 2.3 Hz, 1H), 4.59 (d, J=14.7 Hz, 1H), 4.48 (d, J=14.7 Hz, 1H), 3.69-3.51 (m, 2H), 3.37 (tt, J=10.9, 3.0 Hz, 1H), 2.99-2.82 (m, 1H), 2.70 (td, J=12.2, 2.8 Hz, 1H), 2.59-2.44 (m, 1H), 1.86 (d, J=11.3 Hz, 1H), 1.75-1.63 (m, 3H), 1.47 (s, 6H), 1.35 (s, 9H), 0.85-0.66 (m, 4H). ¹³C NMR (126 MHZ, Chloroform-d) δ 176.89, 173.53, 156.73, 152.41, 144.12, 137.74, 133.29, 129.18, 128.28, 128.02, 126.10, 124.71, 122.99, 110.17, 109.15, 107.60, 81.52, 79.24, 52.25, 49.98, 49.51, 39.85, 29.79, 27.86, 27.83, 25.53, 25.47, 24.48, 9.46, 9.14. HRMS (ESI) Calcd for C34H42N2O4S (M+H)⁺575.2938, found 575.2925. HPLC purity 100%, t_{R}=15.31 min.

N-Cyclopropyl-1-(3-((2-methyl-1-(methylsulfonamido)-1-oxopropan-2-yl)oxy)phenyl)-N-(4-(thiophen-2-yl)benzyl) piperidine-3-carboxamide (18). 1H NMR (500 MHZ, Chloroform-d) δ 8.93 (s, 1H), 7.52-7.45 (m, 2H), 7.22 (dd, J=3.6, 1.2 Hz, 1H), 7.20-7.16 (m, 3H), 7.08 (t, J=8.2 Hz, 1H), 7.00 (dd, J=5.1, 3.5 Hz, 1H), 6.70-6.56 (m, 1H), 6.41 (t, J=2.4 Hz, 1H), 6.28 (dd, J=8.2, 2.2 Hz, 1H), 4.67 (d, J=14.7 Hz, 1H), 4.43 (d, J=14.7 Hz, 1H), 3.68-3.55 (m, 2H), 3.43-3.30 (m, 1H), 3.25 (s, 3H), 2.97 (t, J=11.6 Hz, 1H), 2.73 (t, J=10.6 Hz, 1H), 2.55 (tt, J=7.0, 4.1 Hz, 1H), 1.91 (d, J=9.8 Hz, 1H), 1.81-1.75 (m, 1H), 1.72-1.63 (m, 2H), 1.47 (s, 6H), 0.86-0.74 (m, 4H). ¹³C NMR (126 MHZ, Chloroform-d) δ 176.77, 174.17, 154.14, 152.74, 144.06, 137.64, 133.35, 129.75, 128.29, 128.03, 126.10, 124.75, 123.02, 112.34, 111.53, 109.81, 81.50, 51.89, 49.93, 49.55, 41.32, 39.82, 29.79, 27.80, 24.55, 24.52, 9.64, 8.98. HRMS (ESI) Calcd for C₃₁H37N3O5S2 (M−H) 594.2102, found 594.2107. HPLC purity 97.3%, t_{R}=13.86 min.

1-(3-((1-(Cyclopropanesulfonamido)-2-methyl-1-oxopropan-2-yl)oxy)phenyl)-N-cyclopropyl-N-(4-(thiophen-2-yl)benzyl) piperidine-3-carboxamide (19). 1H NMR (500 MHz, Chloroform-d) δ 8.87 (s, 1H), 7.52-7.46 (m, 2H), 7.23-7.16 (m, 4H), 7.07 (t, J=8.2 Hz, 1H), 7.00 (dd, J=5.1, 3.5 Hz, 1H), 6.67-6.56 (m, 1H), 6.42 (s, 1H), 6.33-6.24 (m, 1H), 4.67 (d, J=14.6 Hz, 1H), 4.42 (d, J=14.7 Hz, 1H), 3.67-3.54 (m, 2H), 3.38 (d, J=11.7 Hz, 1H), 3.01-2.88 (m, 2H), 2.72 (t, J=11.0 Hz, 1H), 2.54 (td, J=6.6, 5.9, 3.4 Hz, 1H), 1.90 (s, 1H), 1.81-1.75 (m, 1H), 1.67 (td, J=11.6, 5.7 Hz, 2H), 1.47 (s, 6H), 1.35-1.32 (m, 2H), 1.08-1.02 (m, 2H), 0.85-0.73 (m, 4H). ¹³C NMR (126 MHz, Chloroform-d) δ 176.78, 173.76, 154.33, 152.74, 144.06, 137.64, 133.34, 129.68, 128.27, 128.04, 126.10, 124.75, 123.02, 112.17, 111.28, 109.61, 81.43, 51.89, 49.98, 49.55, 39.82, 31.06, 29.80, 27.80, 24.65, 24.55, 9.65, 8.98, 6.20. HRMS (ESI) Calcd for C₃₃H39N305S2 (M−H) 620.2258, found 620.2262. HPLC purity 96.9%, t_{R}=14.26 min.

N-Cyclopropyl-1-(3-((2-methyl-1-((1-methylethyl) sulfonamido)-1-oxopropan-2-yl)oxy)phenyl)-N-(4-(thiophen-2-yl)benzyl) piperidine-3-carboxamide (20). ¹H NMR (500 MHZ, Chloroform-d) δ 8.72 (s, 1H), 7.54-7.45 (m, 2H), 7.23-7.16 (m, 4H), 7.07 (t, J=8.2 Hz, 1H), 7.00 (dd, J=5.1, 3.6 Hz, 1H), 6.67-6.56 (m, 1H), 6.42 (s, 1H), 6.29 (dd, J=8.2, 2.2 Hz, 1H), 4.67 (d, J=14.7 Hz, 1H), 4.43 (d, J=14.7 Hz, 1H), 3.77 (hept, J=6.9 Hz, 1H), 3.66-3.56 (m, 2H), 3.37 (t, J=10.7 Hz, 1H), 2.97 (t, J=11.7 Hz, 1H), 2.76-2.68 (m, 1H), 2.59-2.51 (m, 1H), 1.91 (d, J=10.0 Hz, 1H), 1.82-1.74 (m, 1H), 1.68 (td, J=10.2, 8.8, 3.1 Hz, 2H), 1.47 (s, 6H), 1.35 (dd, J=6.9, 1.4 Hz, 6H), 0.86-0.73 (m, 4H). ¹³C NMR (126 MHZ, Chloroform-d) δ 176.79, 174.06, 154.19, 152.72, 144.07, 137.65, 133.33, 129.74, 128.27, 128.03, 126.10, 124.74, 123.02, 112.27, 111.58, 109.76, 81.57, 54.08, 51.93, 49.92, 49.55, 39.82, 29.80, 27.81, 24.64, 24.50, 15.96, 15.93, 9.63, 8.99. HRMS (ESI) Calcd for C33H41N3O5S2 (M−H)⁻622.2415, found 622.2419. HPLC purity 96.9%, t_{R}=14.33 min.

N-Cyclopropyl-1-(3-((2-methyl-1-oxo-1-(phenylsulfonamido) propan-2-yl)oxy)phenyl)-N-(4-(thiophen-2-yl)benzyl) piperidine-3-carboxamide (21). ¹H NMR (500 MHZ, Chloroform-d) δ 9.04 (s, 1H), 8.02-7.94 (m, 2H), 7.64-7.57 (m, 1H), 7.48 (dt, J=7.8, 3.8 Hz, 4H), 7.22-7.16 (m, 4H), 7.03-6.99 (m, 1H), 6.91 (t, J=8.2 Hz, 1H), 6.62-6.51 (m, 1H), 6.35 (t, J=2.5 Hz, 1H), 5.99 (dd, J=8.0, 2.3 Hz, 1H), 4.67 (d, J=14.6 Hz, 1H), 4.43 (d, J=14.7 Hz, 1H), 3.64-3.52 (m, 2H), 3.36 (t, J=10.9 Hz, 1H), 2.94 (t, J=11.7 Hz, 1H), 2.67 (t, J=11.1 Hz, 1H), 2.55 (tt, J=7.2, 4.1 Hz, 1H), 1.90 (d, J=10.8 Hz, 1H), 1.77-1.63 (m, 3H), 1.36 (s, 6H), 0.84-0.74 (m, 4H). ¹³C NMR (126 MHZ, Chloroform-d) δ 176.79, 172.88, 154.30, 152.69, 144.06, 138.17, 137.64, 134.07, 133.34, 129.65, 128.98, 128.49, 128.27, 128.04, 126.11, 124.75, 123.02, 111.98, 110.64, 109.27, 81.21, 51.90, 49.83, 49.56, 39.80, 29.80, 27.80, 24.45, 24.39, 9.64, 8.99. HRMS (ESI) Calcd for C₃₆H39N3O5S2 (M−H) 656.2258, found 656.2252. HPLC purity 96.2%, t_{R}=14.70 min. N-Cyclopropyl-1-(3-((1-((3-methoxyphenyl) sulfonamido)-2-methyl-1-oxopropan-2-yl)oxy)phenyl)-N-(4-(thiophen-2-yl)benzyl) piperidine-3-carboxamide (22). ¹H NMR (500 MHZ, Chloroform-d) δ 9.05 (s, 1H), 7.55 (dt, J=7.8, 1.2 Hz, 1H), 7.50-7.46 (m, 3H), 7.39-7.35 (m, 1H), 7.22-7.15 (m, 4H), 7.11 (ddd, J=8.3, 2.7, 0.9 Hz, 1H), 6.99 (dd, J=5.1, 3.6 Hz, 1H), 6.91 (t, J=8.2 Hz, 1H), 6.56 (dd, J=8.3, 2.3 Hz, 1H), 6.39-6.28 (m, 1H), 5.99 (dd, J=8.1, 2.2 Hz, 1H), 4.66 (d, J=14.7 Hz, 1H), 4.43 (d, J=14.6 Hz, 1H), 3.77 (s, 3H), 3.62-3.52 (m, 2H), 3.35 (dq, J=11.4, 5.8, 4.3 Hz, 1H), 2.94 (t, J=11.6 Hz, 1H), 2.70-2.63 (m, 1H), 2.54 (tt, J=6.9, 4.1 Hz, 1H), 1.89 (d, J=10.9 Hz, 1H), 1.76-1.63 (m, 3H), 1.36 (s, 6H), 0.85-0.73 (m, 4H). ¹³C NMR (126 MHZ, Chloroform-d) δ 176.80, 172.86, 159.68, 154.34, 152.67, 144.06, 139.27, 137.65, 133.34, 129.99, 129.64, 128.26, 128.03, 126.10, 124.75, 123.02, 120.91, 120.55, 112.65, 111.90, 110.56, 109.18, 81.18, 55.73, 51.90, 49.81, 49.56, 39.79, 29.81, 27.79, 24.46, 24.42, 9.62, 8.98. HRMS (ESI) Calcd for C37H41N3O6S2 (M−H) 686.2364, found 686.2360. HPLC purity 99.6%, t_{R}=14.75 min.

N-Cyclopropyl-1-(3-((1-((4-methoxyphenyl) sulfonamido)-2-methyl-1-oxopropan-2-yl)oxy)phenyl)-N-(4-(thiophen-2-yl)benzyl) piperidine-3-carboxamide (23). ¹H NMR (500 MHz, Chloroform-d) δ 9.07 (s, 1H), 8.02-7.95 (m, 2H), 7.56 (d, J=8.0 Hz, 2H), 7.31-7.23 (m, 4H), 7.07 (dd, J=5.1, 3.6 Hz, 1H), 7.02-6.97 (m, 3H), 6.64 (dd, J=8.6, 2.2 Hz, 1H), 6.41 (t, J=2.4 Hz, 1H), 6.08 (dd, J=8.1, 2.2 Hz, 1H), 4.74 (d, J=14.7 Hz, 1H), 4.51 (d, J=14.7 Hz, 1H), 3.89 (s, 3H), 3.65 (t, J=12.7 Hz, 2H), 3.42 (d, J=10.8 Hz, 1H), 3.01 (t, J=11.7 Hz, 1H), 2.75 (t, J=11.1 Hz, 1H), 2.62 (tt, J=7.1, 4.0 Hz, 1H), 1.97 (d, J=10.8 Hz, 1H), 1.83 (d, J=11.1 Hz, 1H), 1.68 (d, J=35.2 Hz, 2H), 1.43 (s, 6H), 0.91-0.82 (m, 4H). ¹³C NMR (126 MHZ, Chloroform-d) δ 176.79, 172.88, 163.99, 154.38, 152.66, 144.07, 137.66, 133.34, 130.88, 129.63, 129.55, 128.63, 128.26, 128.03, 126.10, 124.74, 123.02, 114.12, 111.92, 110.68, 109.20, 81.17, 55.72, 51.90, 49.83, 39.79, 29.81, 27.80, 24.47, 24.43, 9.61, 8.99. HRMS (ESI) Calcd for C37H41N3O6S2 (M−H)⁻686.2364, found 686.2367. HPLC purity 96.5%, t_{R}=14.69 min.

N-Cyclopropyl-1-(3-((1-((3-hydroxyphenyl) sulfonamido)-2-methyl-1-oxopropan-2-yl)oxy)phenyl)-N-(4-(thiophen-2-yl)benzyl) piperidine-3-carboxamide (24). ¹H NMR (500 MHz, Chloroform-d) δ 7.53-7.48 (m, 3H), 7.37 (t, J=2.1 Hz, 1H), 7.29 (t, J=8.0 Hz, 1H), 7.22-7.15 (m, 4H), 7.07-7.04 (m, 1H), 7.00-6.95 (m, 2H), 6.64-6.53 (m, 1H), 6.23 (s, 1H), 6.14 (dd, J=8.0, 2.2 Hz, 1H), 4.72 (d, J=14.7 Hz, 1H), 4.42 (d, J=14.7 Hz, 1H), 3.53-3.38 (m, 3H), 2.86 (t, J=11.5 Hz, 1H), 2.59-2.50 (m, 2H), 1.90 (d, J=8.1 Hz, 1H), 1.75 (dt, J=9.2, 2.9 Hz, 1H), 1.67-1.60 (m, 2H), 1.41 (s, 3H), 1.32 (s, 3H), 0.87-0.77 (m, 4H). ¹³C NMR (126 MHz, Chloroform-d) δ 177.64, 173.00, 157.14, 154.65, 152.70, 143.97, 138.98, 137.07, 133.50, 130.09, 129.78, 128.23, 128.05, 126.18, 124.80, 123.10, 122.21, 120.30, 114.63, 113.10, 112.04, 108.51, 81.33, 51.97, 50.31, 39.93, 30.08, 27.58, 25.38, 24.52, 23.91, 9.71, 8.86. HRMS (ESI) Calcd for C36H39N3O6S2 (M−H)⁻672.2208, found 672.2201. HPLC purity 99.4%, t_{R}=14.07 min. 1-(3-((1-((3-Cyanophenyl) sulfonamido)-2-methyl-1-oxopropan-2-yl)oxy)phenyl)-N-cyclopropyl-N-(4-(thiophen-2-yl)benzyl) piperidine-3-carboxamide (25). ¹H NMR (500 MHz, Chloroform-d) δ 8.30 (dq, J=4.6, 1.4 Hz, 2H), 7.93 (dt, J=7.8, 1.4 Hz, 1H), 7.70 (t, J=8.2 Hz, 1H), 7.58-7.52 (m, 2H), 7.29 (dd, J=3.6, 1.2 Hz, 1H), 7.27-7.23 (m, 3H), 7.07 (dd, J=5.1, 3.6 Hz, 1H), 7.01 (t, J=8.2 Hz, 1H), 6.74-6.62 (m, 1H), 6.42 (s, 1H), 6.12-5.97 (m, 1H), 4.74 (d, J=14.6 Hz, 1H), 4.51 (d, J=14.7 Hz, 1H), 3.70-3.62 (m, 2H), 3.43 (s, 1H), 3.02 (t, J=11.7 Hz, 1H), 2.80-2.71 (m, 1H), 2.62 (tt, J=6.9, 4.1 Hz, 1H), 2.01-1.95 (m, 1H), 1.86-1.81 (m, 1H), 1.74 (td, J=10.7, 9.1, 3.2 Hz, 2H), 1.45 (s, 6H), 0.93-0.82 (m, 4H). ¹³C NMR (126 MHZ, Chloroform-d) δ 176.75, 173.07, 154.15, 152.76, 144.05, 139.78, 137.62, 137.06, 133.34, 132.66, 132.11, 130.02, 129.67, 128.27, 128.03, 126.10, 124.75, 123.02, 116.91, 113.71, 112.17, 110.31, 109.10, 81.17, 51.82, 49.79, 49.56, 39.78, 29.80, 27.78, 24.47, 24.43, 24.38, 9.63, 8.97.

HRMS (ESI) Calcd for C37H38N405S2 (M−H)⁻681.2211, found 681.2219. HPLC purity 98.2%, t_{R}=14.54 min.

1-(3-((1-((4-Cyanophenyl) sulfonamido)-2-methyl-1-oxopropan-2-yl)oxy)phenyl)-N-cyclopropyl-N-(4-(thiophen-2-yl)benzyl) piperidine-3-carboxamide (26). ¹H NMR (500 MHz, Chloroform-d) δ 8.18-8.14 (m, 2H), 7.85-7.82 (m, 2H), 7.58-7.53 (m, 2H), 7.30-7.23 (m, 4H), 7.07 (dd, J=5.1, 3.5 Hz, 1H), 7.01 (t, J=8.2 Hz, 1H), 6.67 (d, J=8.4 Hz, 1H), 6.42 (s, 1H), 6.08 (d, J=8.0 Hz, 1H), 4.74 (d, J=14.7 Hz, 1H), 4.51 (d, J=14.7 Hz, 1H), 3.65 (d, J=11.7 Hz, 2H), 3.40 (d, J=34.3 Hz, 1H), 3.02 (t, J=11.7 Hz, 1H), 2.75 (t, J=11.5 Hz, 1H), 2.62 (tt, J=6.9, 4.1 Hz, 1H), 1.98 (d, J=9.5 Hz, 1H), 1.88-1.83 (m, 1H), 1.74 (dd, J=14.5, 5.8 Hz, 2H), 1.44 (s, 6H), 0.94-0.82 (m, 4H). ¹³C NMR (126 MHz, Chloroform-d) δ 176.74, 173.06, 154.13, 152.81, 144.03, 142.17, 137.60, 133.35, 132.73, 129.67, 129.21, 128.27, 128.04, 126.10, 124.77, 123.03, 117.70, 117.12, 112.28, 110.66, 109.27, 81.26, 51.88, 49.86, 49.57, 39.82, 29.80, 27.74, 24.52, 24.42, 24.35, 9.66, 8.94. HRMS (ESI) Calcd for C37H38N405S2 (M−H)⁻681.2211, found 681.2212. HPLC purity 98.7%, t_{R}=14.62 min.

Ethyl(R)-1-(3-((1-(tert-butoxy)-2-methyl-1-oxopropan-2-yl)oxy)phenyl) piperidine-3-carboxylate (39). ¹H NMR (500 MHZ, Chloroform-d) δ 7.04 (t, J=8.2 Hz, 1H), 6.54 (dd, J=8.1, 2.3 Hz, 1H), 6.45 (t, J=2.4 Hz, 1H), 6.31-6.21 (m, 1H), 4.12 (q, J=7.1 Hz, 2H), 3.67 (ddt, J=12.4, 3.5, 1.5 Hz, 1H), 3.42 (ddd, J=12.3, 4.9, 3.1 Hz, 1H), 2.95 (dd, J=12.4, 9.9 Hz, 1H), 2.80-2.68 (m, 1H), 2.60 (tt, J=10.0, 3.9 Hz, 1H), 2.05-1.93 (m, 1H), 1.74 (th, J=9.2, 3.1 Hz, 1H), 1.69-1.59 (m, 2H), 1.53 (s, 6H), 1.41 (s, 9H), 1.24 (t, J=7.2 Hz, 3H). ¹³C NMR (126 MHZ, Chloroform-d) δ 173.71, 173.43, 156.64, 152.48, 129.08, 110.40, 109.33, 107.56, 81.40, 79.15, 60.40, 52.05, 49.66, 41.34, 27.78, 26.96, 25.49, 25.42, 24.09, 14.24. MS (ESI) m/z=392.3 [M+H]⁺, 414.3 [M+Na]⁺. tert-Butyl(R)-2-(3-(3-((4-bromobenzyl) (cyclopropyl) carbamoyl) piperidin-1-yl)phenoxy)-2-methylpropanoate (40). ¹H NMR (500 MHZ, Chloroform-d) δ 7.46-7.39 (m, 2H), 7.13-7.03 (m, 3H), 6.54 (dd, J=8.3, 2.3 Hz, 1H), 6.47 (t, J=2.3 Hz, 1H), 6.29 (dd, J=8.0, 2.2 Hz, 1H), 4.59 (d, J=14.7 Hz, 1H), 4.49 (d, J=14.7 Hz, 1H), 3.72-3.60 (m, 2H), 3.41 (tt, J=11.0, 3.5 Hz, 1H), 2.96 (dd, J=12.5, 10.9 Hz, 1H), 2.76 (td, J=12.0, 2.9 Hz, 1H), 2.58 (tt, J=7.0, 4.0 Hz, 1H), 1.94-1.89 (m, 1H), 1.79 (ddd, J=12.4, 6.7, 3.4 Hz, 1H), 1.77-1.67 (m, 2H), 1.55 (s, 6H), 1.43 (s, 9H), 0.91-0.75 (m, 4H). MS (ESI) m/z=571.2, 573.2 [M+H]⁺.

tert-Butyl(R)-2-(3-(3-(cyclopropyl(4-(thiophen-2-yl)benzyl)carbamoyl)piperidin-1-yl)phenoxy)-2-methylpropanoate (41). ¹H NMR (500 MHZ, Chloroform-d) δ 7.53-7.42 (m, 2H), 7.24-7.12 (m, 4H), 7.04-6.93 (m, 2H), 6.48 (dd, J=8.2, 2.3 Hz, 1H), 6.41 (t, J=2.3 Hz, 1H), 6.22 (dd, J=8.1, 2.3 Hz, 1H), 4.59 (d, J=14.7 Hz, 1H), 4.48 (d, J=14.7 Hz, 1H), 3.69-3.51 (m, 2H), 3.37 (tt, J=10.9, 3.0 Hz, 1H), 2.99-2.82 (m, 1H), 2.70 (td, J=12.2, 2.8 Hz, 1H), 2.59-2.44 (m, 1H), 1.86 (d, J=11.3 Hz, 1H), 1.75-1.63 (m, 3H), 1.47 (s, 6H), 1.35 (s, 9H), 0.85-0.66 (m, 4H).

(R)—N-Cyclopropyl-1-(3-((1-((3-methoxyphenyl) sulfonamido)-2-methyl-1-oxopropan-2-yl)oxy)phenyl)-N-(4-(thiophen-2-yl)benzyl) piperidine-3-carboxamide (27). ¹H NMR (500 MHZ, Chloroform-d) δ 9.08 (s, 1H), 7.63 (dt, J=7.8, 1.2 Hz, 1H), 7.58-7.53 (m, 3H), 7.45 (t, J=8.0 Hz, 1H), 7.29-7.23 (m, 4H), 7.19 (ddd, J=8.3, 2.6, 0.9 Hz, 1H), 7.07 (dd, J=5.1, 3.6 Hz, 1H), 6.99 (t, J=8.2 Hz, 1H), 6.64 (d, J=8.3 Hz, 1H), 6.42 (s, 1H), 6.12-6.01 (m, 1H), 4.74 (d, J=14.7 Hz, 1H), 4.50 (d, J=14.7 Hz, 1H), 3.85 (s, 3H), 3.69-3.60 (m, 2H), 3.43 (s, 1H), 3.02 (t, J=11.6 Hz, 1H), 2.75 (t, J=11.7 Hz, 1H), 2.62 (tt, J=6.8, 4.0 Hz, 1H), 1.97 (d, J=10.8 Hz, 1H), 1.83 (d, J=11.0 Hz, 1H), 1.75-1.72 (m, 1H), 1.64 (s, 1H), 1.44 (s, 6H), 0.93-0.81 (m, 4H). ¹³C NMR (126 MHZ, Chloroform-d) δ 176.78, 172.82, 159.68, 154.32, 152.68, 144.06, 139.25, 137.64, 133.35, 130.24, 129.99, 129.64, 128.26, 128.03, 126.10, 124.74, 123.02, 120.93, 120.56, 119.18, 118.54, 112.65, 111.92, 111.14, 110.57, 109.20, 81.20, 55.73, 55.68, 51.90, 49.82, 49.56, 39.79, 29.80, 27.79, 24.45, 24.42, 9.62, 8.98. HRMS (ESI) Calcd for C37H41N3O6S2 (M−H)⁻686.2364, found 686.2348. HPLC purity 96.3%, t_{R}=14.70 min.

(R)-1-(3-((1-((4-Cyanophenyl) sulfonamido)-2-methyl-1-oxopropan-2-yl)oxy)phenyl)-N-cyclopropyl-N-(4-(thiophen-2-yl)benzyl) piperidine-3-carboxamide (29). ¹H NMR (500 MHz, Chloroform-d) δ 8.20-8.12 (m, 2H), 7.88-7.81 (m, 2H), 7.62-7.52 (m, 2H), 7.29 (dd, J=3.6, 1.2 Hz, 1H), 7.28-7.26 (m, 1H), 7.24 (d, J=8.1 Hz, 2H), 7.11-6.95 (m, 2H), 6.67 (d, J=8.3 Hz, 1H), 6.42 (s, 1H), 6.08 (d, J=8.0 Hz, 1H), 4.74 (d, J=14.7 Hz, 1H), 4.51 (d, J=14.7 Hz, 1H), 3.65 (d, J=12.1 Hz, 2H), 3.43 (s, 1H), 3.02 (t, J=11.6 Hz, 1H), 2.76 (t, J=11.9 Hz, 1H), 2.62 (tt, J=6.7, 4.1 Hz, 1H), 1.98 (d, J=9.7 Hz, 1H), 1.85 (d, J=9.7 Hz, 1H), 1.73 (d, J=8.7 Hz, 1H), 1.57 (d, J=7.9 Hz, 1H), 1.44 (s, 6H), 0.95-0.82 (m, 4H). ¹³C NMR (126 MHZ, Chloroform-d) δ 176.73, 173.02, 154.10, 144.03, 142.15, 137.59, 133.36, 132.73, 129.68, 129.22, 128.28, 128.04, 126.10, 124.77, 123.03, 117.73, 117.11, 112.30, 110.66, 109.29, 81.28, 51.87, 49.87, 49.57, 39.82, 29.80, 27.75, 24.53, 24.43, 24.35, 9.66, 8.94. HRMS (ESI) Calcd for C37H38N405S2 (M−H)⁻681.2211, found 681.2217. HPLC purity 96.0%, t_{R}=14.62 min.

Ethyl(S)-1-(3-((1-(tert-butoxy)-2-methyl-1-oxopropan-2-yl)oxy)phenyl) piperidine-3-carboxylate (42). ¹H NMR (500 MHZ, Chloroform-d) δ 7.07 (t, J=8.2 Hz, 1H), 6.57 (ddd, J=8.3, 2.4, 0.8 Hz, 1H), 6.48 (t, J=2.4 Hz, 1H), 6.30 (ddd, J=8.1, 2.3, 0.8 Hz, 1H), 4.20-4.11 (m, 2H), 3.70 (ddt, J=12.3, 3.5, 1.5 Hz, 1H), 3.51-3.42 (m, 1H), 2.97 (dd, J=12.4, 10.0 Hz, 1H), 2.77 (tdd, J=10.2, 4.5, 3.1 Hz, 1H), 2.66-2.56 (m, 1H), 2.06-1.95 (m, 1H), 1.82-1.72 (m, 1H), 1.72-1.62 (m, 2H), 1.55 (s, 6H), 1.44 (s, 9H), 1.27 (t, J=7.1 Hz, 3H). ¹³C NMR (126 MHz, Chloroform-d) δ 173.84, 173.52, 156.65, 152.50, 129.10, 110.43, 109.33, 107.63, 81.51, 79.21, 60.47, 52.06, 49.69, 41.41, 27.82, 27.00, 25.51, 25.44, 24.14, 14.26. MS (ESI) m/z=392.2 [M+H]⁺.

tert-Butyl(S)-2-(3-(3-((4-bromobenzyl) (cyclopropyl) carbamoyl) piperidin-1-yl)phenoxy)-2-methylpropanoate (43). ¹H NMR (500 MHZ, CDCl₃) δ 7.37-7.33 (m, 2H), 7.08-6.91 (m, 3H), 6.47 (dd, J=8.1, 2.4 Hz, 1H), 6.40 (t, J=2.3 Hz, 1H), 6.27-6.03 (m, 1H), 4.52 (d, J=14.7 Hz, 1H), 4.47 (s, 1H), 3.63-3.51 (m, 2H), 3.34 (tt, J=10.8, 3.5 Hz, 1H), 2.90 (dd, J=12.5, 10.9 Hz, 1H), 2.69 (td, J=12.1, 2.8 Hz, 1H), 2.51 (tt, J=6.9, 4.1 Hz, 1H), 1.84-1.62 (m, 4H), 1.48 (s, 6H), 1.36 (s, 9H), 0.85-0.65 (m, 4H). MS (ESI) m/z=571.2, 573.2 [M+H]⁺.

tert-Butyl(S)-2-(3-(3-(cyclopropyl(4-(thiophen-2-yl)benzyl)carbamoyl)piperidin-1-yl)phenoxy)-2-methylpropanoate (44). ¹H NMR (500 MHZ, CDCl₃) δ 7.61-7.43 (m, 2H), 7.31-7.19 (m, 4H), 7.10-7.00 (m, 2H), 6.56 (dd, J=7.9, 2.3 Hz, 1H), 6.49 (t, J=2.3 Hz, 1H), 6.33-6.15 (m, 1H), 4.67 (d, J=14.7 Hz, 1H), 4.56 (d, J=14.7 Hz, 1H), 3.77-3.60 (m, 2H), 3.45 (tt, J=11.0, 3.5 Hz, 1H), 3.00 (dd, J=12.4, 11.0 Hz, 1H), 2.77 (td, J=12.2, 2.8 Hz, 1H), 2.70-2.45 (m, 1H), 2.01-1.87 (m, 1H), 1.83-1.74 (m, 2H), 1.55 (s, 6H), 1.43 (s, 9H), 0.97-0.73 (m, 4H).

(S)—N-Cyclopropyl-1-(3-((1-((3-methoxyphenyl) sulfonamido)-2-methyl-1-oxopropan-2-yl)oxy)phenyl)-N-(4-(thiophen-2-yl)benzyl) piperidine-3-carboxamide (28). ¹H NMR (500 MHZ, Chloroform-d) δ 9.06 (s, 1H), 7.63 (ddd, J=7.8, 1.8, 1.0 Hz, 1H), 7.59-7.49 (m, 3H), 7.47-7.43 (m, 1H), 7.29 (dd, J=3.6, 1.2 Hz, 1H), 7.27 (d, J=6.1 Hz, 1H), 7.24 (d, J=8.0 Hz, 2H), 7.19 (ddd, J=8.3, 2.6, 0.9 Hz, 1H), 7.07 (dd, J=5.1, 3.6 Hz, 1H), 6.99 (t, J=8.2 Hz, 1H), 6.64 (dd, J=8.1, 2.4 Hz, 1H), 6.42 (t, J=2.4 Hz, 1H), 6.05 (dd, J=7.8, 2.3 Hz, 1H), 4.74 (d, J=14.7 Hz, 1H), 4.50 (d, J=14.6 Hz, 1H), 3.85 (d, J=1.4 Hz, 3H), 3.69-3.60 (m, 2H), 3.43 (tt, J=11.0, 3.7 Hz, 1H), 3.01 (dd, J=12.4, 11.0 Hz, 1H), 2.75 (td, J=12.2, 2.9 Hz, 1H), 2.66-2.58 (m, 1H), 1.97 (d, J=11.0 Hz, 1H), 1.83 (dq, J=11.8, 3.2, 2.7 Hz, 1H), 1.73 (d, J=9.2 Hz, 1H), 1.62 (d, J=6.9 Hz, 1H), 1.44 (s, 6H), 0.93-0.81 (m, 4H). ¹³C NMR (126 MHZ, CDCl₃) δ 176.80, 172.84, 159.68, 154.29, 152.69, 144.07, 139.21, 137.65, 133.35, 130.02, 129.65, 128.29, 128.06, 126.12, 124.77, 123.03, 120.98, 120.59, 112.61, 111.94, 110.57, 109.22, 81.20, 55.74, 51.88, 49.84, 49.55, 39.79, 29.79, 27.81, 24.46, 24.43, 9.65, 8.99, 0.03. HRMS (ESI) Calcd for C37H41N3O6S2 (M−H)⁻686.2364, found 686.2345. HPLC purity 97.3%, t_{R}=14.69 min.

(S)-((4-Cyanophenyl) sulfonyl) (2-(3-(3-(cyclopropyl(4-(thiophen-2-yl)benzyl) carbamoyl) piperidin-1-yl)phenoxy)-2-methylpropanoyl)amide (30). ¹H NMR (500 MHz, Chloroform-d) δ 8.20-8.12 (m, 2H), 7.88-7.80 (m, 2H), 7.60-7.50 (m, 2H), 7.29 (dd, J=3.5, 1.2 Hz, 1H), 7.28-7.26 (m, 1H), 7.24 (d, J=7.9 Hz, 2H), 7.12-6.97 (m, 2H), 6.67 (d, J=8.3 Hz, 1H), 6.42 (s, 1H), 6.08 (d, J=8.0 Hz, 1H), 4.74 (d, J=14.7 Hz, 1H), 4.51 (d, J=14.7 Hz, 1H), 3.65 (dd, J=11.8, 3.6 Hz, 2H), 3.45 (d, J=12.3 Hz, 1H), 3.02 (t, J=11.6 Hz, 1H), 2.76 (t, J=11.6 Hz, 1H), 2.62 (tt, J=6.9, 4.1 Hz, 1H), 1.98 (d, J=9.9 Hz, 1H), 1.89-1.81 (m, 1H), 1.75 (d, J=8.9 Hz, 1H), 1.62 (s, 1H), 1.44 (s, 6H), 0.93-0.81 (m, 4H). ¹³C NMR (126 MHz, Chloroform-d) δ 176.73, 173.02, 154.10, 152.83, 144.03, 142.15, 137.60, 133.36, 132.73, 129.68, 129.22, 128.28, 128.04, 126.10, 124.77, 123.03, 117.73, 117.11, 112.30, 110.66, 109.28, 81.28, 51.87, 49.87, 39.82, 29.80, 27.75, 24.53, 24.43, 24.35, 9.66, 8.94. HRMS (ESI) Calcd for C37H38N405S2 (M−H) 681.2211, found 681.2199. HPLC purity 97.0%, t_{R}=14.61 min.

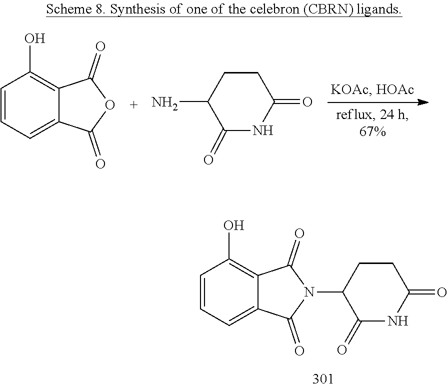 [SMILES: O=C1OC(=O)c2c(O)cccc21 ; NC1CCC(=O)NC1=O ; O=C1CCC(N2C(=O)c3cccc(O)c3C2=O)C(=O)N1]

2-(2,6-dioxopiperidin-3-yl)-4-hydroxyisoindoline-1,3-dione (301). In a round-bottom flask, 3-hydroxyphthalic anhydride (2.0 g, 12.2 mmol, 1.0 eq), KOAc (3.72 g, 37.82 mmol, 3.1 eq) and 3-aminoperidine-2,6-dione hydrochloride (2.2 g, 13.4 mmol, 1.1 eq) were mixed in acetic acid (40 mL). The resulting reaction mixture was heated to reflux for 24 h. After cooling to ambient temperature, solvent was evaporated and washed with water to yield brown solid which was purified by recrystallization with MeOH with to give the desired product 301 (2.23 g, 67%). ¹H NMR (500 MHZ, DMSO-d₆) δ 11.17 (s, 1H), 11.08 (s, 1H), 7.65 (dd, J=8.4, 7.2 Hz, 1H), 7.36-7.29 (m, 1H), 7.25 (dd, J=8.4, 0.8 Hz, 1H), 5.07 (dd, J=12.8, 5.5 Hz, 1H), 2.88 (ddd, J=16.9, 13.8, 5.5 Hz, 1H), 2.62-2.54 (m, 2H), 2.02 (ddq, J=10.5, 5.5, 3.1, 2.5 Hz, 1H).¹³C NMR (126 MHz, DMSO-d₆) δ 170.01, 167.02, 165.81, 155.46, 136.39, 133.16, 123.56, 114.37, 114.30, 48.65, 30.97, 22.04.

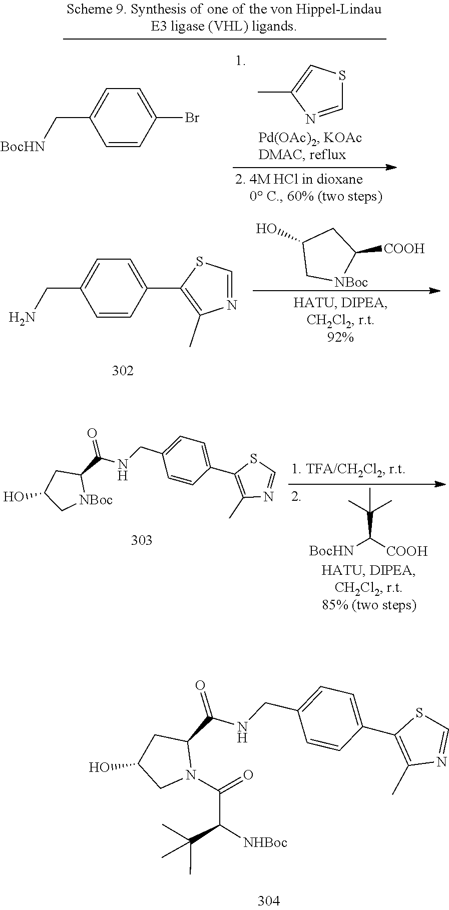

(4-(4-methylthiazol-5-yl)phenyl) methanamine (302). A solution of tert-butyl(4-bromobenzyl) carbamate (1 eq), 4-methylthiazole (2 eq), Pd(OAc) 2 (1%), and KOAc (2 eq) in N,N-dimethylacetamide (DMAc) was refluxed under Ar overnight. The mixture was concentrated and purified by flash chromatography to yield pure compound, which was added to 4 M HCl in dioxane and stirred at 0° C. for 4 h. The mixture was concentrated and ethyl acetate and saturated NaHCO₃were added. The organic phase was washed by brine and dried over Na₂SO₄ and concentrated to yield compound 302 (yield 60%). ¹H NMR (500 MHz, CD₃OD) δ 8.87 (s, 1H), 7.43 (s, 4H), 3.84 (s, 2H), 2.47 (s, 3H). MS (ESI) 205.2 (M+H)⁺.

tert-butyl(2S,4R)-4-hydroxy-2-((4-(4-methylthiazol-5-yl)benzyl) carbamoyl) pyrrolidine-1-carboxylate (303). To a solution of 302 (1 eq) and (2S,4R)-1-(tert-butoxycarbonyl)-4-hydroxypyrrolidine-2-carboxylic acid (1 eq) in CH₂Cl₂ were added HATU (1.5 eq) and DIPEA (2 eq). The mixture was stirred at room temperature overnight. Water was added to the mixture and the organic phase was concentrated and purified by flash chromatography to yield 303 (yield 92%). ¹H NMR (400 MHZ, CDCl₃) δ 8.67 (s, 1H), 7.42-7.29 (m, 4H), 4.48 (d, J=16.7 Hz, 4H), 3.51 (dd, J=11.0, 4.7 Hz, 2H), 2.59-2.44 (m, 4H), 2.03 (d, J=7.4 Hz, 2H), 1.43 (s, 9H). MS (ESI) 418.3 (M+H)+.

tert-butyl ((S)-1-((2S,4R)-4-hydroxy-2-((4-(4-methylthiazol-5-yl)benzyl) carbamoyl) pyrrolidin-1-yl)-3,3-dimethyl-1-oxobutan-2-yl) carbamate (304). Compound 303 (1 eq) was dissolved in TFA/CH₂Cl₂ (1/10) and stirred at room temperature for 6 h. The mixture was concentrated and redissolved in CH₂Cl₂. (S)-2-((tert-butoxycarbonyl)amino)-3,3-dimethylbutanoic acid (1 eq), HATU (1.5 eq), and DIPEA (2 eq) were added to the mixture and stirred at room temperature overnight. Water was added and organic phase was concentrated and purified by flash chromatography to produce 304 (yield 85%). ¹H NMR (500 MHZ, CDCl₃) δ 8.61 (s, 1H), 7.28 (q, J=8.2 Hz, 4H), 5.08 (d, J=9.0 Hz, 1H), 4.71 (t, J=7.9 Hz, 1H), 4.57-4.41 (m, 2H), 4.25 (dd, J=14.9, 5.1 Hz, 1H), 4.06 (t, J=10.8 Hz, 2H), 3.49 (dd, J=11.4, 3.5 Hz, 1H), 2.55 (ddd, J=12.8, 7.8, 4.6 Hz, 1H), 2.45 (s, 3H), 2.06 (dd, J=13.6, 8.3 Hz, 1H), 1.34 (s, 9H), 0.84 (s, 9H).

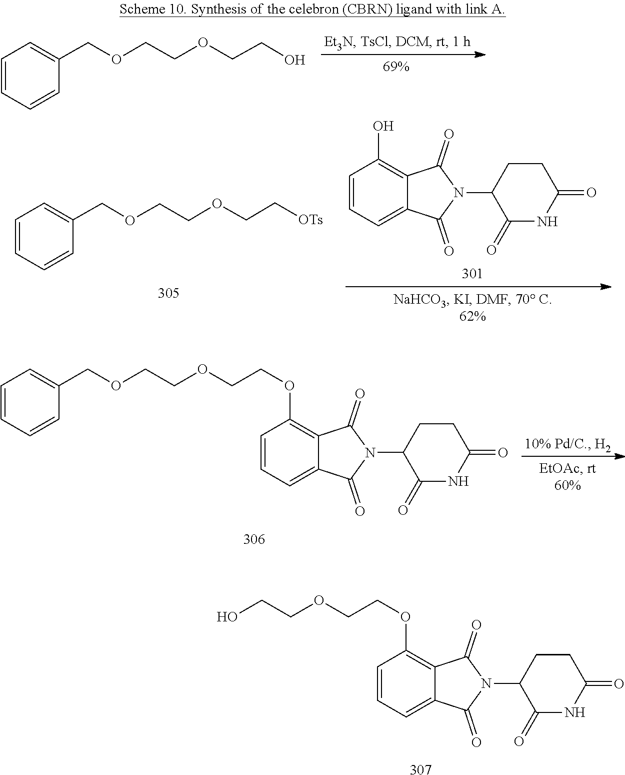 [SMILES: OCCOCCOCc1ccccc1 ; Cc1ccc(S(=O)(=O)OCCOCCOCc2ccccc2)cc1 ; O=C1CCC(N2C(=O)c3cccc(O)c3C2=O)C(=O)N1 ; O=C1CCC(N2C(=O)c3cccc(OCCOCCOCc4ccccc4)c3C2=O)C(=O)N1 ; O=C1CCC(N2C(=O)c3cccc(OCCOCCO)c3C2=O)C(=O)N1 ; "307"]

2-(2-(benzyloxy) ethoxy)ethyl 4-methylbenzenesulfonate (305). To a solution of alcohol (1.0 eq) was added Et₃N (3.0 eq) and TsCl (1.5 eq) at 0° C. The mixture was stirred at room temperature for 1 h. Then mixture was concentrated under reduced pressure to remove the solvent. The residue was purified by column chromatography. The title compound 305 (2.44 g, 69%) was synthesized. ¹H NMR (500 MHZ, Chloroform-d) δ 7.79 (d, J=8.3 Hz, 2H), 7.37-7.25 (m, 7H), 4.53 (s, 2H), 4.20-4.14 (m, 2H), 3.72-3.67 (m, 2H), 3.63-3.59 (m, 2H), 3.59-3.54 (m, 2H), 2.42 (s, 3H).¹³C NMR (126 MHZ, Chloroform-d) δ 144.77, 138.12, 133.00, 129.81, 128.39, 127.97, 127.72, 127.65, 73.28, 70.81, 69.37, 69.27, 68.70, 21.63.

4-(2-(2-(benzyloxy) ethoxy) ethoxy)-2-(2,6-dioxopiperidin-3-yl) isoindoline-1,3-dione (306). To a solution of compound 301 (0.14 g, 0.5 mmol), compound 305 (1.0 mmol) in DMF (4.0 mL) was added NaHCO₃ (84 mg, 1.0 mmol) and KI (10 mg). The reaction mixture was stirred at 70° C. for 12 h prior to being taken up in ethyl acetate and water. The organic layer was separated, dried, and evaporated. The residue was purified by column chromatography to afford the desired product. The title compound 306 (0.14 g, 62%) was synthesized. ¹H NMR (500 MHZ, Chloroform-d) δ 8.11 (s, 1H), 7.37 (dd, J=8.5, 7.3 Hz, 1H), 7.18 (dd, J=7.3, 0.7 Hz, 1H), 7.07-7.05 (m, 3H), 7.02-6.98 (m, 2H), 4.68 (dd, J=12.3, 5.4 Hz, 1H), 4.29 (s, 2H), 4.09 (dd, J=5.3, 4.3 Hz, 2H), 3.72-3.65 (m, 2H), 3.57-3.50 (m, 2H), 3.42-3.35 (m, 2H), 2.63-2.40 (m, 3H), 1.87-1.76 (m, 1H). ¹³C NMR (126 MHz, Chloroform-d) δ 171.13, 168.17, 167.01, 165.59, 156.46, 138.23, 136.44, 133.73, 128.35, 127.75, 127.60, 119.62, 117.32, 116.14, 73.23, 71.19, 69.54, 69.45, 69.35, 49.10, 31.38, 22.59.

2-(2,6-dioxopiperidin-3-yl)-4-(2-(2-hydroxyethoxy) ethoxy) isoindoline-1,3-dione (307). To a solution of alcohol with compound 306 in ethyl acetate was added 10% Pd/C (0.1 eq, weight). After evacuating and flushing the flask with hydrogen gas three times, the reaction mixture was stirred under hydrogen atmosphere for 3 h. Upon completion, Pd/C was removed via filtration. The filtrate was concentrated under vacuum and the crude product was purified via column chromatography to give the desired product. The title compound 307 (0.024 g, 60%) was synthesized. ¹H NMR (500 MHZ, Chloroform-d) δ 8.39 (s, 1H), 7.67 (dd, J=8.5, 7.3 Hz, 1H), 7.46 (dd, J=7.3, 0.7 Hz, 1H), 7.23 (dd, J=8.5, 0.7 Hz, 1H), 4.97 (dd, J=12.4, 5.3 Hz, 1H), 4.37-4.30 (m, 2H), 4.00-3.95 (m, 2H), 3.77 (t, J=4.8 Hz, 2H), 3.75-3.70 (m, 2H), 2.90-2.70 (m, 4H), 2.13-2.07 (m, 1H). ¹³C NMR (126 MHz, Chloroform-d) δ 171.12, 168.20, 166.98, 165.93, 156.37, 136.61, 133.78, 119.23, 117.40, 116.29, 72.75, 69.05, 68.98, 61.77, 49.19, 31.42, 22.61.

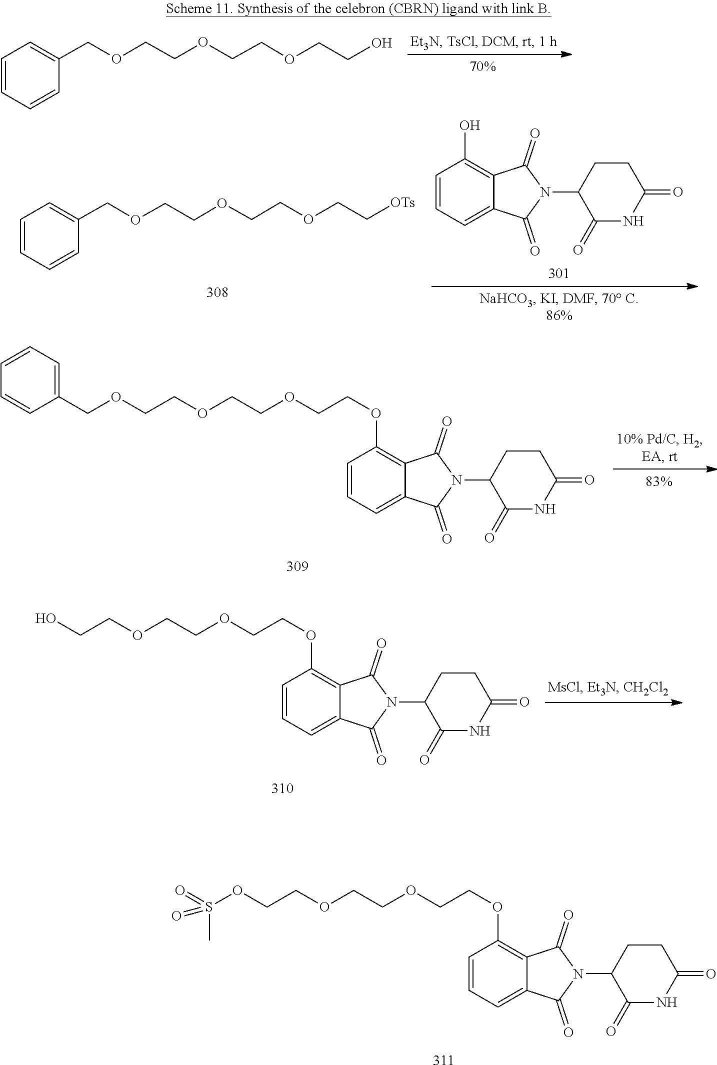

2-(2-(2-(benzyloxy) ethoxy) ethoxy)ethyl 4-methylbenzenesulfonate (308). To a solution of alcohol (1.0 eq) was added Et₃N (3.0 eq) and TsCl (1.5 eq) at 0° C. The mixture was stirred at room temperature for 1 h. Then mixture was concentrated under reduced pressure to remove the solvent. The residue was purified by column chromatography. The title compound 308 (5.5 g, 70%) was synthesized. ¹H NMR (500 MHZ, Chloroform-d) δ 7.70-7.64 (m, 2H), 7.24-7.14 (m, 7H), 4.44 (s, 2H), 4.06-4.01 (m, 2H), 3.59-3.55 (m, 2H), 3.54-3.51 (m, 2H), 3.51-3.48 (m, 2H), 3.47 (s, 4H), 2.31 (s, 3H). ¹³C NMR (126 MHZ, Chloroform-d) δ 144.78, 138.25, 133.04, 129.82, 128.38, 127.99, 127.75, 127.62, 73.25, 70.79, 70.70, 70.60, 69.43, 69.26, 68.71, 21.65.

4-(2-(2-(2-(benzyloxy) ethoxy) ethoxy) ethoxy)-2-(2,6-dioxopiperidin-3-yl) isoindoline-1,3-dione (309). To a solution of compound 301 (0.14 g, 0.5 mmol), compound 308 (1.0 mmol) in DMF (4.0 mL) was added NaHCO₃ (84 mg, 1.0 mmol) and KI (10 mg). The reaction mixture was stirred at 70° C. for 12 h prior to being taken up in ethyl acetate and water. The organic layer was separated, dried, and evaporated. The residue was purified by column chromatography to afford the desired product. The title compound 309 (1.5 g, 86%) was synthesized. ¹H NMR (500 MHZ, Chloroform-d) δ 8.09 (s, 1H), 7.67 (dd, J=8.5, 7.3 Hz, 1H), 7.49-7.46 (m, 1H), 7.36-7.34 (m, 4H), 7.30-7.28 (m, 2H), 5.01-4.90 (m, 1H), 4.58 (s, 2H), 4.37 (dd, J=5.7, 4.0 Hz, 2H), 3.99-3.94 (m, 2H), 3.83-3.78 (m, 2H), 3.71-3.68 (m, 4H), 3.67-3.63 (m, 3H), 2.93-2.72 (m, 3H), 2.16-2.09 (m, 1H). ¹³C NMR (126 MHz, Chloroform-d) δ 170.87, 167.99, 167.01, 165.59, 156.52, 138.31, 136.48, 133.77, 128.39, 128.37, 127.80, 127.76, 127.63, 119.64, 117.36, 116.18, 73.26, 71.19, 70.75, 70.68, 69.48, 69.46, 69.36, 53.47, 49.14, 31.42, 22.64.

2-(2,6-dioxopiperidin-3-yl)-4-(2-(2-(2-hydroxyethoxy) ethoxy) ethoxy) isoindoline-1,3-dione (310). To a solution of compound 309 in ethyl acetate was added 10% Pd/C (0.1 eq, weight). After evacuating and flushing the flask with hydrogen gas three times, the reaction mixture was stirred under hydrogen atmosphere for 3 h. Upon completion, Pd/C was removed via filtration. The filtrate was concentrated under vacuum and the crude product was purified via column chromatography to give the desired product. The title compound 310 (1.02 g, 83%) was synthesized. ¹H NMR (500 MHZ, Chloroform-d) δ 8.22 (s, 1H), 7.67 (dd, J=8.5, 7.3 Hz, 1H), 7.47 (dd, J=7.3, 0.7 Hz, 1H), 7.28-7.24 (m, 2H), 4.95 (dd, J=12.3, 5.4 Hz, 1H), 4.39-4.33 (m, 2H), 3.99-3.92 (m, 2H), 3.83-3.78 (m, 2H), 3.75-3.66 (m, 4H), 3.64-3.58 (m, 2H), 2.92-2.69 (m, 3H), 2.54 (t, J=6.2 Hz, 1H), 2.15-2.08 (m, 1H). ¹³C NMR (126 MHZ, Chloroform-d) δ 170.98, 168.12, 166.99, 165.72, 156.42, 136.55, 133.81, 119.47, 117.40, 116.28, 72.51, 71.26, 70.44, 69.36, 61.80, 49.18, 31.43, 22.66.

2-(2-(2-((2-(2,6-dioxopiperidin-3-yl)-1,3-dioxoisoindolin-4-yl)oxy) ethoxy) ethoxy)ethyl methanesulfonate (311). To a solution of compound 310 (0.04 g, 0.1 mmol) and MsCl (23 mg, 0.2 mmol) in dry DCM (2.0 mL) was added TEA (30 mg, 0.3 mmol) dropwise at 0° C. Then the mixture was stirred for 1 h at 0° C. The reaction mixture was stirred at 0° C. for 1 h prior to being taken up in DCM and water. The organic layer was separated, dried, and evaporated. The residue was purified by column chromatography to afford the desired product 311 (0.05 g). ¹H NMR (500 MHZ, Chloroform-d) δ 8.48 (s, 1H), 7.67 (dd, J=8.5, 7.2 Hz, 1H), 7.46 (dd, J=7.3, 0.7 Hz, 1H), 7.24 (d, J=8.5 Hz, 1H), 4.95 (dd, J=12.0, 5.4 Hz, 1H), 4.37-4.30 (m, 4H), 3.95-3.90 (m, 2H), 3.81-3.75 (m, 4H), 3.71-3.67 (m, 2H), 3.04 (s, 3H), 2.89-2.69 (m, 3H), 2.11 (ddd, J=7.8, 5.8, 2.1 Hz, 1H). ¹³C NMR (126 MHz, Chloroform-d) δ 171.21, 168.34, 166.98, 165.61, 156.32, 136.54, 133.82, 119.26, 117.30, 116.19, 71.17, 70.54, 69.41, 69.27, 69.21, 69.01, 49.15, 37.61, 31.38, 22.63.

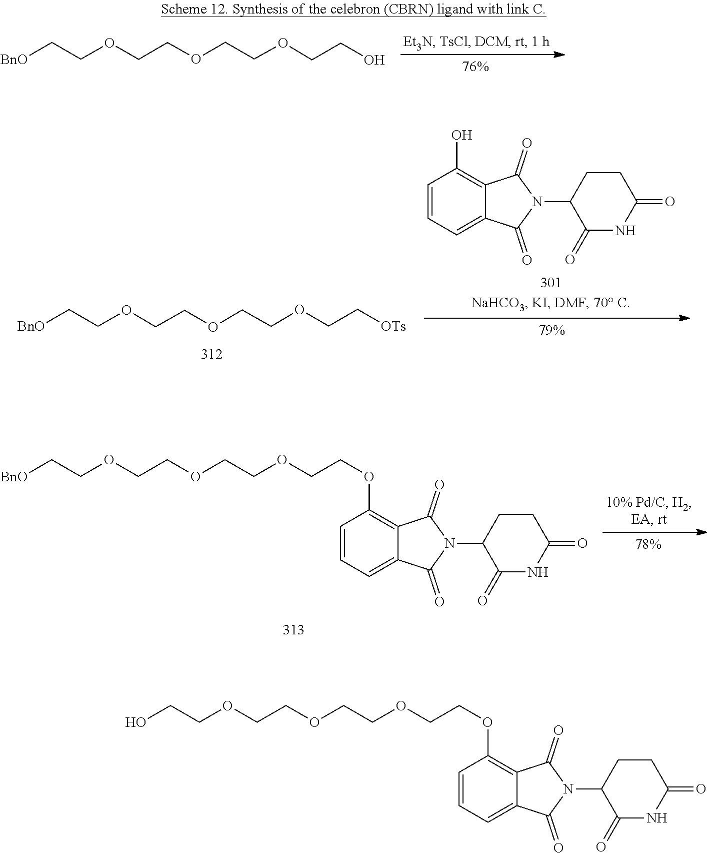 [SMILES: OCCOCCOCCOCCOCc1ccccc1 ; O=C1CCC(N2C(=O)c3cccc(O)c3C2=O)C(=O)N1 ; Cc1ccc(S(=O)(=O)OCCOCCOCCOCCOCc2ccccc2)cc1 ; O=C1CCC(N2C(=O)c3cccc(OCCOCCOCCOCCOCc4ccccc4)c3C2=O)C(=O)N1 ; O=C1CCC(N2C(=O)c3cccc(OCCOCCOCCOCCO)c3C2=O)C(=O)N1]

1-phenyl-2,5,8,11-tetraoxatridecan-13-yl 4-methylbenzenesulfonate (312). To a solution of alcohol (1.0 eq) was added Et₃N (3.0 eq) and TsCl (1.5 eq) at 0° C. The mixture was stirred at room temperature for 1 h. Then mixture was concentrated under reduced pressure to remove the solvent. The residue was purified by column chromatography. The title compound 312 (4.99 g, 75.85%) was synthesized. ¹H NMR (500 MHZ, Chloroform-d) δ 7.83-7.75 (m, 2H), 7.36-7.30 (m, 6H), 7.30-7.26 (m, 1H), 4.56 (s, 2H), 4.17-4.11 (m, 2H), 3.69-3.65 (m, 4H), 3.65-3.61 (m, 6H), 3.60-3.55 (m, 4H), 2.43 (s, 3H). ¹³C NMR (126 MHZ, Chloroform-d) δ 144.78, 138.31, 133.08, 129.82, 128.37, 128.00, 127.75, 127.60, 73.26, 70.78, 70.67, 70.66, 70.57, 69.48, 69.26, 68.70, 21.65.

2-(2,6-dioxopiperidin-3-yl)-4-((1-phenyl-2,5,8,11-tetraoxatridecan-13-yl)oxy) isoindoline-1,3-dione (313). To a solution of compound 301 (0.14 g, 0.5 mmol), compound 312 (1.0 mmol) in DMF (4.0 mL) was added NaHCO₃ (84 mg, 1.0 mmol) and KI (10 mg). The reaction mixture was stirred at 70° C. for 12 h prior to being taken up in ethyl acetate and water. The organic layer was separated, dried, and evaporated. The residue was purified by column chromatography to afford the desired product. The title compound 313 (1.51 g, 79%) was synthesized. ¹H NMR (500 MHZ, Chloroform-d) δ 8.21 (s, 1H), 7.65 (dd, J=8.4, 7.3 Hz, 1H), 7.45 (dd, J=7.3, 0.7 Hz, 1H), 7.35-7.31 (m, 4H), 7.27-7.24 (m, 2H), 4.93 (dd, J=12.3, 5.4 Hz, 1H), 4.55 (d, J=2.2 Hz, 2H), 4.36-4.31 (m, 2H), 3.95-3.91 (m, 2H), 3.79-3.75 (m, 2H), 3.68-3.64 (m, 8H), 3.64-3.61 (m, 2H), 2.90-2.68 (m, 3H), 2.14-2.07 (m, 1H). ¹³C NMR (126 MHz, Chloroform-d) δ 170.94, 168.04, 167.01, 165.57, 156.50, 138.29, 136.46, 133.78, 128.38, 128.36, 127.77, 127.75, 127.61, 119.63, 117.36, 116.16, 73.24, 71.18, 70.69, 70.66, 70.64, 70.60, 69.49, 69.42, 69.35, 49.13, 31.41, 22.65.

2-(2,6-dioxopiperidin-3-yl)-4-(2-(2-(2-(2-hydroxyethoxy) ethoxy) ethoxy) ethoxy) isoindoline-1,3-dione (314). To a solution of compound 313 in ethyl acetate was added 10% Pd/C (0.1 eq, weight). After evacuating and flushing the flask with hydrogen gas three times, the reaction mixture was stirred under hydrogen atmosphere for 3 h. Upon completion, Pd/C was removed via filtration. The filtrate was concentrated under vacuum and the crude product was purified via column chromatography to give the desired product. The title compound 314 (0.98 g, 78%) was synthesized. ¹H NMR (500 MHZ, Chloroform-d) δ 8.34 (s, 1H), 7.67 (dd, J=8.5, 7.3 Hz, 1H), 7.46 (d, J=7.3 Hz, 1H), 7.27 (d, J=8.8 Hz, 1H), 4.95 (dd, J=12.2, 5.3 Hz, 1H), 4.38-4.33 (m, 2H), 3.94 (dd, J=5.3, 4.2 Hz, 2H), 3.82-3.77 (m, 2H), 3.72-3.64 (m, 8H), 3.62-3.57 (m, 2H), 2.91-2.69 (m, 3H), 2.66 (d, J=6.5 Hz, 1H), 2.16-2.08 (m, 1H). 13C NMR (126 MHz, Chloroform-d) δ 171.06, 168.17, 167.02, 165.64, 156.48, 136.50, 133.80, 119.55, 117.38, 116.19, 72.50, 71.18, 70.62, 70.59, 70.38, 69.38, 61.75, 49.16, 31.42, 22.68.

Protein Expression and Purification. Full-length β-catenin (residues 1-781) were cloned into a pET-28b vector carrying a C-terminal 6× histidine (Novagen), and transformed into E. coli BL21 DE3 (Novagen). Cells were cultured in LB medium with 50 μg/mL kanamycin until the OD600 was approximately 0.8, and then protein expression was induced with 400 μM of IPTG at 20° C. overnight. Cells were lysed by sonication. The proteins were purified by three steps of chromatography, including Ni-NTA affinity chromatography (30210, Qiagen), HiTrap Q HP anion exchange chromatography (17-1154-01, GE Healthcare Life Science) and size-exclusion chromatography with a HiLoad 26/600 Superdex 200 pg column (28-9893-36, GE Healthcare Life Science) using an AKTA Pure FPLC (GE Healthcare Life Science) system. Protein was eluted in a buffer containing 20 mM of Tris (pH 8.5), 100 mM NaCl, and 2 mM DTT. The purity of β-catenin was greater than 95% as determined by SDS-PAGE gel analyses. Thermal shift assays were performed on an CFX96 Real Time System (Bio-Rad) to monitor protein stability and detect protein aggregation. Protein unfolding was evaluated through measurement of the fluorescence changes of fluorescent dye Sypro Orange when interacting with β-catenin proteins. A temperature increment of 1°/min was applied. All proteins were stable and no aggregation was observed under storage or assay conditions. Proteins were aliquoted and stored at −80° C.

BCL9 Peptide Synthesis and Purification. Human BCL9 (residues 350-375) and N-terminally biotinylated human BCL9 (residues 350-375) were synthesized by InnoPep Inc. (San Diego, CA, www.innopep.com). The synthesized peptides were purified by HPLC with purity >95%. The structures were validated by LC/MS. The sequences of the peptides are as follows (Ahx, 6-aminohexanoic acid).

| Peptide | Sequence |
|---|---|
| BCL9 26-mer | H-³⁵⁰GLSQEQLEHRERSLQTLRDIQRMLFP³⁷⁵-NH₂ |
| | (SEQ ID NO. 1) |
| Biotinylated | Biotin-Ahx-³⁵⁰GLSQEQLEHRERSLQTLRDIQRM |
| BCL9 26-mer | LFP³⁷⁵-NH₂ |
| | (SEQ ID NO. 2) |

AlphaScreen Assays of β-Catenin and BCL9 Interaction. Experiments were performed in white opaque 384-well plates from PerkinElmer (Waltham, MA), and the samples were read on a Biotek Synergy 2 plate reader (Winooski, VT) with excitation at 680 nm and emission at 570 nm. The standard AlphaScreen protocol was used with a sensitivity setting of 200. All dilutions were made in 1x assay buffer containing 25 mM Hepes (pH 7.4), 100 mM NaCl, 0.01% Triton X-100 and 0.1% BSA to minimize nonspecific interactions. For the competitive inhibition assays of the β-catenin/BCL9 PPI, the negative control (equivalent to 0% inhibition) refers to 5.0 nM biotinylated BCL9, 50 nM His6-tagged β-catenin, and 10 μg/mL donor and acceptor beads in a final volume of 25 μL assay buffer, but no tested inhibitor present. The positive control (equivalent to 100% inhibition) refers to 5.0 nM biotinylated BCL9 and 10 μg/mL donor and acceptor beads in a final volume of 25 μL assay buffer.

For the β-catenin/BCL9 assay, 5 nM biotinylated BCL9, and 50 nM His6-tagged 8-catenin were incubated in assay buffer for 30 min. Different concentrations of the tested inhibitor were added and incubated in 20 μL assay buffer for another 1 h. All of the assay plates were covered and gently mixed on an orbital shaker. Donor and acceptor beads were then added to the plates to a final concentration of 10 μg/mL in 25 μL assay buffer. The mixture was incubated for 1 h before detection. The IC₅₀ value was determined by nonlinear least-square analysis of GraphPad Prism 8.0. The Kᵢ values were derived from the IC₅₀ values using a method reported by Wang and coworkers.⁴⁶ The equation used is Kᵢ= [I]₅₀/([L]₅₀/K_{d}+[P]₀/K_{d}+1) (Where [I]₅₀ denotes the concentration of the free inhibitor at 50% inhibition, [L]₅₀ is the concentration of the free labeled ligand at 50% inhibition, [P]₀ is the concentration of the free protein at 0% inhibition, and K_{d} is the dissociation constant of the protein-ligand complex). All of the experiments were performed in triplicate and carried out in the presence of 1% DMSO for small-molecule inhibitors. Each compound was assayed at least by three independent experiments. The results were expressed as mean±standard deviation.

C₀-Immunoprecipitation (C₀-IP) Experiments. HCT116 cancer cells with hyperactive 8-catenin signaling at 1×10⁶ cells/mL were treated with different concentrations of the inhibitor for 24 h. Cells were then lysed in buffer containing 50 mM Tris, pH 7.4, 150 mM NaCl, 1% Nonidet P-40, 2 mM EDTA, and protease inhibitors. The cell lysates were pre-adsorbed to A/G plus agarose (sc-2003, Santa Cruz Biotechnology) at 4° C. for 1 h. Pre-adsorbed lysates were incubated with a specific primary antibody against β-catenin (610153, BD Biosciences) overnight at 4° C. A/G plus agarose was then added to the lysate mixture and incubated for 3 h. The beads were washed four times with the lysis buffer at 4° C. The bound protein was eluted by boiling in the SDS sample buffer and loaded onto 8% SDS polyacrylamide gel for electrophoretic analysis. Separated proteins were transferred onto nitrocellulose membranes for immunoblot analysis. The antibodies against BCL9 (ab37305, Abcam) and E-cadherin (610404, BD Biosciences), were incubated with the membranes, respectively. IRDye 680LT goat anti mouse IgG (827-11080, LiCOR) and IRDye 800CW goat anti rabbit IgG (926-32211, LiCOR) were used as the secondary antibodies. The images were detected by the Odyssey Infrared Imaging System (LiCOR). Experiments were performed in triplicate.

Cell Transfection and Luciferase Assay. FuGENE6 (E269A, Promega) 96 well plate format was used for the transfection of SW480 cells according to the manufacturer's instruction. SW480 cells were co-transfected with 60 ng the TOPFlash or FOPFlash firefly luciferase reporter gene and 40 ng renilla luciferase pCMV-RL normalization reporter gene. Cells were cultured in DMEM and 10% FBS at 37° C. for 24 h, and different concentrations of inhibitors or DMSO was added. After 24 h, the luciferase reporter activity was measured using the Dual-Glo system (E2940, Promega). Normalized luciferase activity in response to the treatment with inhibitors was compared with that obtained from the cells treated with DMSO. Experiments were performed in triplicate.

Quantitative Real Time PCR analysis. SW480 cells at 1×10⁶ cells/mL were treated with inhibitors at different concentrations for 24 h. MDA-MB-231 cells at 1×10⁶ cells/mL were treated with 100 ng/mL recombinant Wnt 3a (rmW3aL-010, Time BioScience) for 30 min, then the inhibitors at different concentration were added and incubated for 24 h. Total RNAs were extracted with TRIzol (15596026, Life Technologies), and the cDNA was synthesized with the superscript III first-strand kit (18080-051, Invitrogen). qPCR experiments were performed using the iQTM SYBR green supermix kit (170-8880, BIO-RAD) on an CFX96 Real Time System (BIO-RAD). The threshold cycle (CT) values were normalized to that of internal reference GAPDH. Experiments were performed in triplicate. The primer pairs are shown below

| | | |
|---|---|---|
| human | forward | 5′-GAAGGTGAAGGTCGGAGTC-3′ |
| GAPDH | | (SEQ ID NO. 3) |
| | reverse | 5′-GAAGATGGTGATGGGATTTC-3′ |
| | | (SEQ ID NO. 4) |
| human | forward | 5′-GCTATAAATTCTTTGCTGACCTGCTG-3′ |
| HPRT | | (SEQ ID NO. 5) |
| | reverse | 5′-AATTACTTTTATGTCCCCTGTTGACTGG-3′ |
| | | (SEQ ID NO. 6) |
| human | forward | 5′-AGTGTGAGGTCCACGGAAAC-3′ |
| AXIN2 | | (SEQ ID NO. 7) |
| | reverse | 5′-CTTCACACTGCGATGCATTT-3′ |
| | | (SEQ ID NO. 8) |
| human | forward | 5′-GACGAGATGATCCCCTTCAA-3′ |
| LEF1 | | (SEQ ID NO. 9) |
| | reverse | 5′-AGGGCTCCT GAGAGGTTTGT-3′ |
| | | (SEQ ID NO. 10) |
| human | forward | 5′-ACAAACAGATCATCCGCAAACAC-3′ |
| cyclin D1 | | (SEQ ID NO. 11) |
| | reverse | 5′-TGTTGGGGCTCCTCAGGTTC-3′ |
| | | (SEQ ID NO. 12) |

Western Blotting of Wnt Target Genes. SW480 cells at 1×10⁶ cells/mL were treated with different concentrations of inhibitors for 24 h. Cells were lysed in buffer containing 50 mM Tris, pH 7.4, 150 mM NaCl, 1% Nonidet P-40, 2 mM EDTA, and protease inhibitors. After centrifugation at 12,000 rpm for 20 min at 4° C., the supernatant was loaded onto an 8% SDS polyacrylamide gel for electrophoretic analysis. Separated proteins were transferred onto nitrocellulose membranes for immunoblot analysis. The antibodies against Axin2 (MA5-15015, Thermo Fisher), cyclin D1 (sc-853, Santa Cruz Biotechnology), and β-tubulin (sc-55529, Santa Cruz Biotechnology) were incubated with the membranes overnight at 4° C., respectively. IRDye 680LT goat anti-mouse IgG (827-11080, LiCOR) or IRDye 800CW goat anti-rabbit IgG (827-08365, LiCOR) was used as the secondary antibody. The images were detected by the Odyssey Infrared Imaging System (Odyssey Fc, LiCOR). Experiments were performed in duplicate.

MTS Cell Growth Inhibition Experiments. Colorectal cancer cells (SW480 and HCT116) and triple negative breast cancer cells (MDA-MB-231 and MDA-MB-468) were seeded in 96-well plates at 5×10³ cells/well in DMEM with 10% FCS, maintained overnight at 37° C., and incubated with the tested compounds at various concentrations. Human mammary epithelial cell MCF10A were seeded in 96-well plates at 1×10⁴ cells/well in MEGM (CC-3150, Lonza) with 100 ng/mL cholera toxin, maintained overnight at 37° C., and incubated with the tested compounds at various concentrations. Cell viability was monitored after 72 h using a freshly prepared mixture of 1 part phenazine methosulfate (PMS, Sigma) solution (0.92 mg/mL) and 19 parts 3-(4,5-dimethylthiazol-2-yl)-5-(3-carboxymethoxyphenyl)-2-(4-sulfophenyl)-2H-tetrazolium (MTS, Promega) solution (2 mg/mL). Cells were incubated in 10 μL of this solution at 37° C. for 3 h, and A490 was measured. The effect of each compound is expressed as the concentration required to reduce A490 by 50% (IC₅₀) relative to DMSO-treated cells. Experiments were performed in triplicate.

### Abbreviations

BCL9, B-cell lymphoma 9; PPI, protein-protein interaction; APC, adenomatous polyposis coli; GSK38, glycogen synthase kinase 38; CK1α, casein kinase 1α; PP2A, protein phosphatase 2A; TCF, T-cell factor; LEF, lymphoid enhancer-binding factor; BCL9L, BCL9-like; Pygo, Pygopus; CBP, CREB-binding protein; Fzd, Frizzled; GPCRs, G-protein-coupled receptors; Dvl, Dishevelled; PNPB, 3-(4-fluorophenyl)-N-phenylbenzamide; Kᵢ, inhibition constant; qPCR, quantitative real-time PCR; co-IP, co-immunoprecipitation; PAINS, pan-assay interference compounds.

### REFERENCES

(1) Nusse, R.; Clevers, H. Wnt/β-Catenin Signaling, Disease, and Emerging Therapeutic Modalities. Cell 2017, 169, 985-999.
(2) Anastas, J. N.; Moon, R. T. WNT signalling pathways as therapeutic targets in cancer. Nature reviews. Cancer 2013, 13, 11-26.
(3) Rubinfeld, B.; Albert, I.; Porfiri, E.; Fiol, C.; Munemitsu, S.; Polakis, P. Binding of GSK3beta to the APC-beta-catenin complex and regulation of complex assembly. Science (New York, N. Y.) 1996, 272, 1023-1026.
(4) Clevers, H.; Nusse, R. Wnt/β-catenin signaling and disease. Cell 2012, 149, 1192-1205.
(5) Polakis, P. Wnt signaling in cancer. Cold Spring Harbor perspectives in biology 2012, 4.
(6) van de Wetering, M.; Cavallo, R.; Dooijes, D.; van Beest, M.; van Es, J.; Loureiro, J.; Ypma, A.; Hursh, D.; Jones, T.; Bejsovec, A.; Peifer, M.; Mortin, M.; Clevers, H. Armadillo coactivates transcription driven by the product of the Drosophila segment polarity gene dTCF. Cell 1997, 88, 789-799.
(7) Hecht, A.; Vleminckx, K.; Stemmler, M. P.; van Roy, F.; Kemler, R. The p300/CBP acetyltransferases function as transcriptional coactivators of beta-catenin in vertebrates. The EMBO journal 2000, 19, 1839-1850.
(8) Bienz, M.; Clevers, H. Linking colorectal cancer to Wnt signaling. Cell 2000, 103, 311-320.
(9) Nakajima, M.; Fukuchi, M.; Miyazaki, T.; Masuda, N.; Kato, H.; Kuwano, H. Reduced expression of Axin correlates with tumour progression of oesophageal squamous cell carcinoma. British journal of cancer 2003, 88, 1734-1739.
(10) Liu, W.; Dong, X.; Mai, M.; Seelan, R. S.; Taniguchi, K.; Krishnadath, K. K.; Halling, K. C.; Cunningham, J. M.; Boardman, L. A.; Qian, C.; Christensen, E.; Schmidt, S. S.; Roche, P. C.; Smith, D. I.; Thibodeau, S. N. Mutations in AXIN2 cause colorectal cancer with defective mismatch repair by activating beta-catenin/TCF signalling. Nature genetics 2000, 26, 146-147.
(11) Kahn, M. Can we safely target the WNT pathway? Nature reviews. Drug discovery 2014, 13, 513-532.
(12) Teeuwssen, M.; Fodde, R. Wnt Signaling in Ovarian Cancer Stemness, EMT, and Therapy Resistance. Journal of clinical medicine 2019, 8.
(13) Herr, P.; Hausmann, G.; Basler, K. WNT secretion and signalling in human disease. Trends in molecular medicine 2012, 18, 483-493.
(14) Bafico, A.; Liu, G.; Goldin, L.; Harris, V.; Aaronson, S. A. An autocrine mechanism for constitutive Wnt pathway activation in human cancer cells. Cancer cell 2004, 6, 497-506.
(15) Caldwell, G. M.; Jones, C.; Gensberg, K.; Jan, S.; Hardy, R. G.; Byrd, P.; Chughtai, S.; Wallis, Y.; Matthews, G. M.; Morton, D. G. The Wnt antagonist sFRP1 in colorectal tumorigenesis. Cancer research 2004, 64, 883-888.
(16) Suzuki, H.; Watkins, D. N.; Jair, K. W.; Schuebel, K. E.; Markowitz, S. D.; Chen, W. D.; Pretlow, T. P.; Yang, B.; Akiyama, Y.; Van Engeland, M.; Toyota, M.; Tokino, T.; Hinoda, Y.; Imai, K.; Herman, J. G.; Baylin, S. B. Epigenetic inactivation of SFRP genes allows constitutive WNT signaling in colorectal cancer. Nature genetics 2004, 36, 417-422.
(17) Fukui, T.; Kondo, M.; Ito, G.; Maeda, O.; Sato, N.; Yoshioka, H.; Yokoi, K.; Ueda, Y.; Shimokata, K.; Sekido, Y. Transcriptional silencing of secreted frizzled related protein 1 (SFRP 1) by promoter hypermethylation in non-small-cell lung cancer. Oncogene 2005, 24, 6323-6327.
(18) Milovanovic, T.; Planutis, K.; Nguyen, A.; Marsh, J. L.; Lin, F.; Hope, C.; Holcombe, R. F. Expression of Wnt genes and frizzled 1 and 2 receptors in normal breast epithelium and infiltrating breast carcinoma. International journal of oncology 2004, 25, 1337-1342.
(19) Spranger, S.; Bao, R.; Gajewski, T. F. Melanoma-intrinsic β-catenin signalling prevents anti-tumour immunity. Nature 2015, 523, 231-235.
(20) Luke, J. J.; Bao, R.; Sweis, R. F.; Spranger, S.; Gajewski, T. F. WNT/β-catenin Pathway Activation Correlates with Immune Exclusion across Human Cancers. Clinical cancer research: an official journal of the American Association for Cancer Research 2019, 25, 3074-3083.
(21) Grasso, C. S.; Giannakis, M.; Wells, D. K.; Hamada, T.; Mu, X. J.; Quist, M.; Nowak, J. A.; Nishihara, R.; Qian, Z. R.; Inamura, K.; Morikawa, T.; Nosho, K.; Abril-Rodriguez, G.; Connolly, C.; Escuin-Ordinas, H.; Geybels, M. S.; Grady, W. M.; Hsu, L.; Hu-Lieskovan, S.; Huyghe, J. R.; Kim, Y. J.; Krystofinski, P.; Leiserson, M. D. M.; Montoya, D. J.; Nadel, B. B.; Pellegrini, M.; Pritchard, C. C.; Puig-Saus, C.; Quist, E. H.; Raphael, B. J.; Salipante, S. J.; Shin, D. S.; Shinbrot, E.; Shirts, B.; Shukla, S.; Stanford, J. L.; Sun, W.; Tsoi, J.; Upfill-Brown, A.; Wheeler, D. A.; Wu, C. J.; Yu, M.; Zaidi, S. H.; Zaretsky, J. M.; Gabriel, S. B.; Lander, E. S.; Garraway, L. A.; Hudson, T. J.; Fuchs, C. S.; Ribas, A.; Ogino, S.; Peters, U. Genetic Mechanisms of Immune Evasion in Colorectal Cancer. Cancer discovery 2018, 8, 730-749.
(22) Gurney, A.; Axelrod, F.; Bond, C. J.; Cain, J.; Chartier, C.; Donigan, L.; Fischer, M.; Chaudhari, A.; Ji, M.; Kapoun, A. M.; Lam, A.; Lazetic, S.; Ma, S.; Mitra, S.; Park, I. K.; Pickell, K.; Sato, A.; Satyal, S.; Stroud, M.; Tran, H.; Yen, W. C.; Lewicki, J.; Hoey, T. Wnt pathway inhibition via the targeting of Frizzled receptors results in decreased growth and tumorigenicity of human tumors. Proceedings of the National Academy of Sciences of the United States of America 2012,109, 11717-11722.
(23) Chen, B.; Dodge, M. E.; Tang, W.; Lu, J.; Ma, Z.; Fan, C. W.; Wei, S.; Hao, W.; Kilgore, J.; Williams, N. S.; Roth, M. G.; Amatruda, J. F.; Chen, C.; Lum, L. Small molecule-mediated disruption of Wnt-dependent signaling in tissue regeneration and cancer. Nature chemical biology 2009, 5, 100-107.
(24) Liu, J.; Pan, S.; Hsieh, M. H.; Ng, N.; Sun, F.; Wang, T.; Kasibhatla, S.; Schuller, A. G.; Li, A. G.; Cheng, D.; Li, J.; Tompkins, C.; Pferdekamper, A.; Steffy, A.; Cheng, J.; Kowal, C.; Phung, V.; Guo, G.; Wang, Y.; Graham, M. P.; Flynn, S.; Brenner, J. C.; Li, C.; Villarroel, M. C.; Schultz, P. G.; Wu, X.; McNamara, P.; Sellers, W. R.; Petruzzelli, L.; Boral, A. L.; Seidel, H. M.; Mclaughlin, M. E.; Che, J.; Carey, T. E.; Vanasse, G.; Harris, J. L. Targeting Wnt-driven cancer through the inhibition of Porcupine by LGK974. Proceedings of the National Academy of Sciences of the United States of America 2013, 110, 20224-20229.
(25) Huang, S. M.; Mishina, Y. M.; Liu, S.; Cheung, A.; Stegmeier, F.; Michaud, G. A.; Charlat, O.; Wiellette, E.; Zhang, Y.; Wiessner, S.; Hild, M.; Shi, X.; Wilson, C. J.; Mickanin, C.; Myer, V.; Fazal, A.; Tomlinson, R.; Serluca, F.; Shao, W.; Cheng, H.; Shultz, M.; Rau, C.; Schirle, M.; Schlegl, J.; Ghidelli, S.; Fawell, S.; Lu, C.; Curtis, D.; Kirschner, M. W.; Lengauer, C.; Finan, P. M.; Tallarico, J. A.; Bouwmeester, T.; Porter, J. A.; Bauer, A.; Cong, F. Tankyrase inhibition stabilizes axin and antagonizes Wnt signalling. Nature 2009, 461, 614-620.
(26) Thorne, C. A.; Hanson, A. J.; Schneider, J.; Tahinci, E.; Orton, D.; Cselenyi, C. S.; Jernigan, K. K.; Meyers, K. C.; Hang, B. I.; Waterson, A. G.; Kim, K.; Melancon, B.; Ghidu, V. P.; Sulikowski, G. A.; LaFleur, B.; Salic, A.; Lee, L. A.; Miller, D. M., 3rd; Lee, E. Small-molecule inhibition of Wnt signaling through activation of casein kinase 1α. Nature chemical biology 2010, 6, 829-836.
(27) Cheong, J. K.; Nguyen, T. H.; Wang, H.; Tan, P.; Voorhoeve, P. M.; Lee, S. H.; Virshup, D. M. IC261 induces cell cycle arrest and apoptosis of human cancer cells via CK1δ/∈ and Wnt/β-catenin independent inhibition of mitotic spindle formation. Oncogene 2011, 30, 2558-2569.
(28) Mani, M.; Carrasco, D. E.; Zhang, Y.; Takada, K.; Gatt, M. E.; Dutta-Simmons, J.; Ikeda, H.; Diaz-Griffero, F.; Pena-Cruz, V.; Bertagnolli, M.; Myeroff, L. L.; Markowitz, S. D.; Anderson, K. C.; Carrasco, D. R. BCL9 promotes tumor progression by conferring enhanced proliferative, metastatic, and angiogenic properties to cancer cells. Cancer research 2009, 69, 7577-7586.
(29) Deka, J.; Wiedemann, N.; Anderle, P.; Murphy-Seiler, F.; Bultinck, J.; Eyckerman, S.; Stehle, J. C.; André, S.; Vilain, N.; Zilian, O.; Robine, S.; Delorenzi, M.; Basler, K.; Aguet, M. Bc19/Bc191 are critical for Wnt-mediated regulation of stem cell traits in colon epithelium and adenocarcinomas. Cancer research 2010, 70, 6619-6628.
(30) Brembeck, F. H.; Wiese, M.; Zatula, N.; Grigoryan, T.; Dai, Y.; Fritzmann, J.; Birchmeier, W. BCL9-2 promotes early stages of intestinal tumor progression. Gastroenterology 2011, 141, 1359-1370, 1370.e1351-1353.
(31) Gay, D. M.; Ridgway, R. A.; Müller, M.; Hodder, M. C.; Hedley, A.; Clark, W.; Leach, J. D.; Jackstadt, R.; Nixon, C.; Huels, D. J.; Campbell, A. D.; Bird, T. G.; Sansom, O. J. Loss of BCL9/91 suppresses Wnt driven tumourigenesis in models that recapitulate human cancer. Nat Commun 2019, 10, 723.
(32) Kawamoto, S. A.; Coleska, A.; Ran, X.; Yi, H.; Yang, C. Y.; Wang, S. Design of triazole-stapled BCL9 a-helical peptides to target the β-catenin/B-cell CLL/lymphoma 9 (BCL9) protein-protein interaction. J Med Chem 2012, 55, 1137-1146.
(33) Takada, K.; Zhu, D.; Bird, G. H.; Sukhdeo, K.; Zhao, J. J.; Mani, M.; Lemieux, M.; Carrasco, D. E.; Ryan, J.; Horst, D.; Fulciniti, M.; Munshi, N. C.; Xu, W.; Kung, A. L.; Shivdasani, R. A.; Walensky, L. D.; Carrasco, D. R. Targeted disruption of the BCL9/β-catenin complex inhibits oncogenic Wnt signaling. Science translational medicine 2012, 4, 148ra117.
(34) Feng, M.; Jin, J. Q.; Xia, L.; Xiao, T.; Mei, S.; Wang, X.; Huang, X.; Chen, J.; Liu, M.; Chen, C.; Rafi, S.; Zhu, A. X.; Feng, Y. X.; Zhu, D. Pharmacological inhibition of β-catenin/BCL9 interaction overcomes resistance to immune checkpoint blockades by modulating T (reg) cells. Science advances 2019, 5, eaau5240.
(35) Sang, P.; Zhang, M.; Shi, Y.; Li, C.; Abdulkadir, S.; Li, Q.; Ji, H.; Cai, J. Inhibition of β-catenin/B cell lymphoma 9 protein-protein interaction using a-helix-mimicking sulfono-y-AApeptide inhibitors. Proceedings of the National Academy of Sciences of the United States of America 2019, 116, 10757-10762.
(36) de la Roche, M.; Rutherford, T. J.; Gupta, D.; Veprintsev, D. B.; Saxty, B.; Freund, S. M.; Bienz, M. An intrinsically labile a-helix abutting the BCL9-binding site of β-catenin is required for its inhibition by carnosic acid. Nat Commun 2012, 3, 680.
(37) de la Roche, M.; Ibrahim, A. E.; Mieszczanek, J.; Bienz, M. LEF1 and B9L shield β-catenin from inactivation by Axin, desensitizing colorectal cancer cells to tankyrase inhibitors. Cancer research 2014, 74, 1495-1505.
(38) Hoggard, L. R.; Zhang, Y.; Zhang, M.; Panic, V.; Wisniewski, J. A.; Ji, H. Rational design of selective small-molecule inhibitors for β-catenin/B-cell lymphoma 9 protein-protein interactions. Journal of the American Chemical Society 2015, 137, 12249-12260.
(39) Zhang, M.; Wang, Z.; Zhang, Y.; Guo, W.; Ji, H. Structure-Based Optimization of Small-Molecule Inhibitors for the β-Catenin/B-Cell Lymphoma 9 Protein-Protein Interaction. J Med Chem 2018, 61, 2989-3007.
(40) Wisniewski, J. A.; Yin, J.; Teuscher, K. B.; Zhang, M.; Ji, H. Structure-Based Design of 1,4-Dibenzoylpiperazines as β-Catenin/B-Cell Lymphoma 9 Protein-Protein Interaction Inhibitors. ACS Med Chem Lett 2016, 7, 508-513.
(41) Zhang, M.; Wisniewski, J. A.; Ji, H. AlphaScreen selectivity assay for-catenin/B-cell lymphoma 9 inhibitors. Analytical biochemistry 2015, 469, 43-53.
(42) Vacondio, F.; Silva, C.; Mor, M.; Testa, B. Qualitative structure-metabolism relationships in the hydrolysis of carbamates. Drug metabolism reviews 2010, 42, 551-589.
(43) Bagley, S. W.; Brandt, T. A.; Dugger, R. W.; Hada, W. A.; Hayward, C. M.; Liu, Z. Preparation of phenyl substituted piperidines as PPAR agonists, in particular PPARα agonists, and their pharmaceutical compositions and therapeutic use as hypolipemics, antidiabetics, etc. WO2004048334, 2004.
(44) Ammazzalorso, A.; De Filippis, B.; Giampietro, L.; Amoroso, R. N-acylsulfonamides: Synthetic routes and biological potential in medicinal chemistry. Chemical biology & drug design 2017, 90, 1094-1105.
(45) Sampietro, J.; Dahlberg, C. L.; Cho, U. S.; Hinds, T. R.; Kimelman, D.; Xu, W. Crystal structure of a β-catenin/BCL9/Tcf4 complex. Mol. Cell 2006, 24, 293-300.
(46) Nikolovska-Coleska, Z.; Wang, R.; Fang, X.; Pan, H.; Tomita, Y.; Li, P.; Roller, P. P.; Krajewski, K.; Saito, N. G.; Stuckey, J. A.; Wang, S. Development and optimization of a binding assay for the XIAP BIR3 domain using fluorescence polarization. Analytical biochemistry 2004, 332, 261-273.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

1. A compound of Formula I: 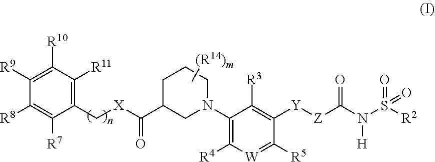
or a pharmaceutically acceptable salt thereof;
wherein:
W is CR⁶ or N;
X is selected from O or NR¹;
Y is absent or O;
Z is absent or C(R¹²)(R¹³);
m is 0, 1, or 2;
n is 1 or 2;
R¹ is selected from hydrogen, C₁-C₆alkyl, C₁-C₆haloalkyl, C₃-C₇cycloalkyl, 4- to 10-membered monocyclic or bicyclic heterocycle, 5- to 10-membered monocyclic or bicyclic aryl, 5- to 10-membered monocyclic or bicyclic heteroaryl, and R^{L}, each of which may be optionally substituted with one or more A groups as allowed by valency;
R² is selected from C₁-C₆alkyl, C₃-C₇cycloalkyl, (4- to 10-membered monocyclic and bicyclic heterocycle)-(C₀-C₆alkyl), (5- to 10-membered monocyclic or bicyclic aryl)-(C₀-C₆alkyl), (5- to 10-membered monocyclic or bicyclic heteroaryl)-(C₀-C₆alkyl), and R^{L}, each of which may be optionally substituted with one or more B groups as allowed by valency;
R³, R⁴, RS, and R⁶ are independently selected from hydrogen, halo, nitro, cyano, azido, C₁-C₆alkyl, C₁-C₆haloalkyl, C₂-C₆alkenyl, C₂-C₆alkynyl, C₃-C₇cycloalkyl, (4- to 10-membered monocyclic or bicyclic heterocycle)-(C₀-C₆alkyl), (5- to 10-membered monocyclic or bicyclic aryl)-(C₀-C₆alkyl), (5- to 10-membered monocyclic or bicyclic heteroaryl)-(C₀-C₆alkyl), —OR^{a}, —SR^{a}, —NR^{a}R^{b}, —C(O)R^{c}, —S(O)R^{c}, —S(O)₂R^{c}, and R^{L}, each of which may be optionally substituted with one or more A groups as allowed by valency;
R⁷, R⁸, R⁹, R¹⁰, and R¹¹ are independently selected from hydrogen, halo, nitro, cyano, azido, C₁-C₆alkyl, C₁-C₆haloalkyl, C₂-C₆alkenyl, C₂-C₆alkynyl, C₃-C₇cycloalkyl, (4- to 10-membered monocyclic or bicyclic heterocycle)-(C₀-C₆alkyl), (5- to 10-membered monocyclic or bicyclic aryl)-(C₀-C₆alkyl), (5- to 10-membered monocyclic or bicyclic heteroaryl)-(C₀-C₆alkyl), —OR^{a}, —SR^{a}, —NR^{a}R^{b}, —C(O)R^{c}, —S(O)R^{c}, —S(O)₂Re, and R^{L}, each of which may be optionally substituted with one or more A groups as allowed by valency;
R¹² and R¹³ are independently selected from hydrogen, halo, nitro, cyano, azido, C₁-C₀alkyl, C₁-C₆haloalkyl, C₂-C₆alkenyl, C₂-C₆alkynyl, C₃-C₇cycloalkyl, (4- to 10-membered monocyclic or bicyclic heterocycle)-(C₀-C₆alkyl), (5- to 10-membered monocyclic or bicyclic aryl)-(C₀-C₆alkyl), (5- to 10-membered monocyclic or bicyclic heteroaryl)-(C₀-C₆alkyl), —OR^{a}, —SR^{a}, —NR^{a}R^{b}, —C(O)R^{c}, —S(O)R^{c}, —S(O)₂R^{c}, and R^{L}, each of which may be optionally substituted with one or more A groups as allowed by valency;
or R¹² and R₁₃ are brought together with the carbon to which they are attached to form a C₃-C₇ cycloalkyl group or a 4- to 10-membered monocyclic or bicyclic heterocycle group;
R¹⁴ is independently selected at each occurrence from halo, nitro, cyano, azido, C₁-C₆alkyl, C₁-C₆haloalkyl, C₂-C₆alkenyl, C₂-C₆alkynyl, C₃-C₇cycloalkyl, (4- to 10-membered monocyclic or bicyclic heterocycle)-(C₀-C₆alkyl), (5- to 10-membered monocyclic or bicyclic aryl)-(C₀-C₆alkyl), (5- to 10-membered monocyclic or bicyclic heteroaryl)-(C₀-C₆alkyl), —OR^{a}, —SR^{a}, —NR^{a}R^{b}, —C(O)R^{c}, —S(O)R^{c}, —S(O)₂R^{c} and R^{L};
R^{a} and R^{b} are independently selected at each occurrence from hydrogen, C₁-C₆alkyl, C₁-C₆haloalkyl, C₂-C₆alkenyl, C₂-C₆alkynyl, C₃-C₇cycloalkyl, (4- to 10-membered monocyclic or bicyclic heterocycle)-(C₀-C₆alkyl), (5- to 10-membered monocyclic or bicyclic aryl)-(C₀-C₆alkyl), and (5- to 10-membered monocyclic or bicyclic heteroaryl)-(C₀-C₆alkyl), each of which may be optionally substituted with one or more A groups as allowed by valency;
Re is independently selected at each occurrence from hydrogen, halo, C₁-C₆alkyl, C₁-C₆haloalkyl, C₂-C₆alkenyl, C₂-C₆alkynyl, C₃-C₇cycloalkyl, (4- to 10-membered monocyclic or bicyclic heterocycle)-(C₀-C₆alkyl), (5- to 10-membered monocyclic or bicyclic aryl)-(C₁-C₆alkyl), (5- to 10-membered monocyclic or bicyclic heteroaryl)-(C₀-C₆alkyl), —OR^{a}, —SR^{a}, and —NR^{a}R^{b}, each of which may be optionally substituted with one or more A groups as allowed by valency;
A is selected at each occurrence from halo, nitro, cyano, azido, C₁-C₆alkyl, C₁-C₆haloalkyl, C₂-C₆alkenyl, C₂-C₆alkynyl, C₃-C₇cycloalkyl, (4- to 10-membered monocyclic or bicyclic heterocycle)-(C₀-C₆alkyl), (5- to 10-membered monocyclic or bicyclic aryl)-(C₀-C₆alkyl), (5- to 10-membered monocyclic or bicyclic heteroaryl)-(C₀-C₆alkyl), C₁-C₆alkoxy, C₁-C₆haloalkoxy, thiol, C₁-C₆thioalkyl, amino, (mono- or dialkyl)amino, —CHO, —COOH, —CONH₂, ester, carbamate, urea, sulfonamide, phosphate, phosphonate, and R^{L};
B is selected from halo, nitro, cyano, azido, C₁-C₆alkyl, C₁-C₆haloalkyl, C₂-C₆alkenyl, C₂-C₆alkynyl, C₃-C₇cycloalkyl, (4- to 10-membered monocyclic or bicyclic heterocycle)-(C₀-C₆alkyl), (5- to 10-membered monocyclic or bicyclic aryl)-(C₀-C₆alkyl), (5- to 10-membered monocyclic or bicyclic heteroaryl)-(C₀-C₆alkyl), —OR^{a}, —SR^{a}, —NR^{a}R^{b}, —C(O)R^{c}, —S(O)R^{c}, —S(O)₂R^{c}, and R^{L}, each of which may be optionally substituted with one or more A groups as allowed by valency; and
R^{L} is a linker conjugated to a PROTAC moiety.

2. The compound of claim 1, wherein W is CR⁶.

3. The compound of claim 1, wherein X is NR¹.

4. The compound of claim 1, wherein Y is O.

5. The compound of claim 1, wherein Z is C(R¹²)(R¹³).

6. The compound of claim 1, wherein R⁹ is 5- to 10-membered monocyclic or bicyclic heteroaryl.

7. The compound of claim 1, wherein n is 1.

8. The compound of claim 1, wherein m is 0.

9. The compound of claim 1, wherein the compound is of Formula I-a or Formula I-b: 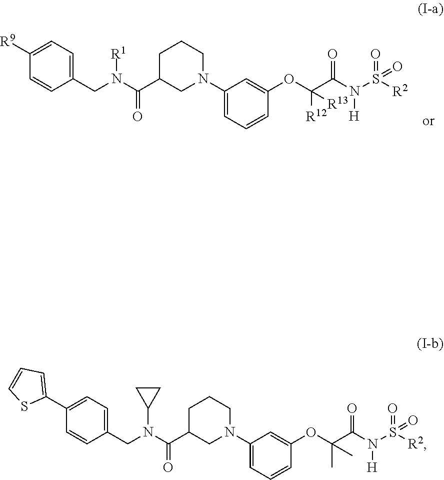
or a pharmaceutically acceptable salt thereof.

10. (canceled)

11. The compound of claim 1, wherein R² is C₁-C₀alkyl, C₃-C₇cycloalkyl, or phenyl optionally substituted with one or more groups selected from hydroxyl, cyano, or C₁-C₆alkoxy.

12-13. (canceled)

14. The compound of claim 1, wherein the linker conjugated to a PROTAC moiety is comprises a linker selected from substituted or unsubstituted C₄-C₂₄alkyl, a substituted or unsubstituted C₄-C₂₄alkoxy, and the PROTAC moiety is selected from a cereblon binder or a von Hippel-Lindau E3 ligase (VHL) ligand.

15. The compound of claim 1, wherein the compound is selected from the group consisting of: 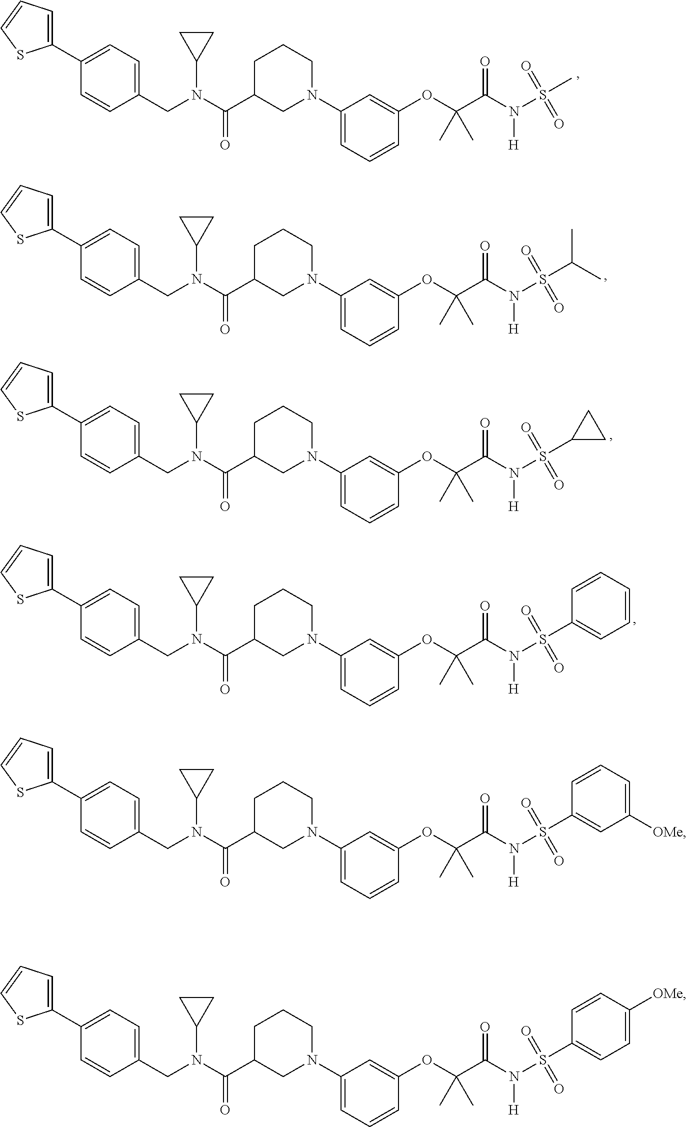 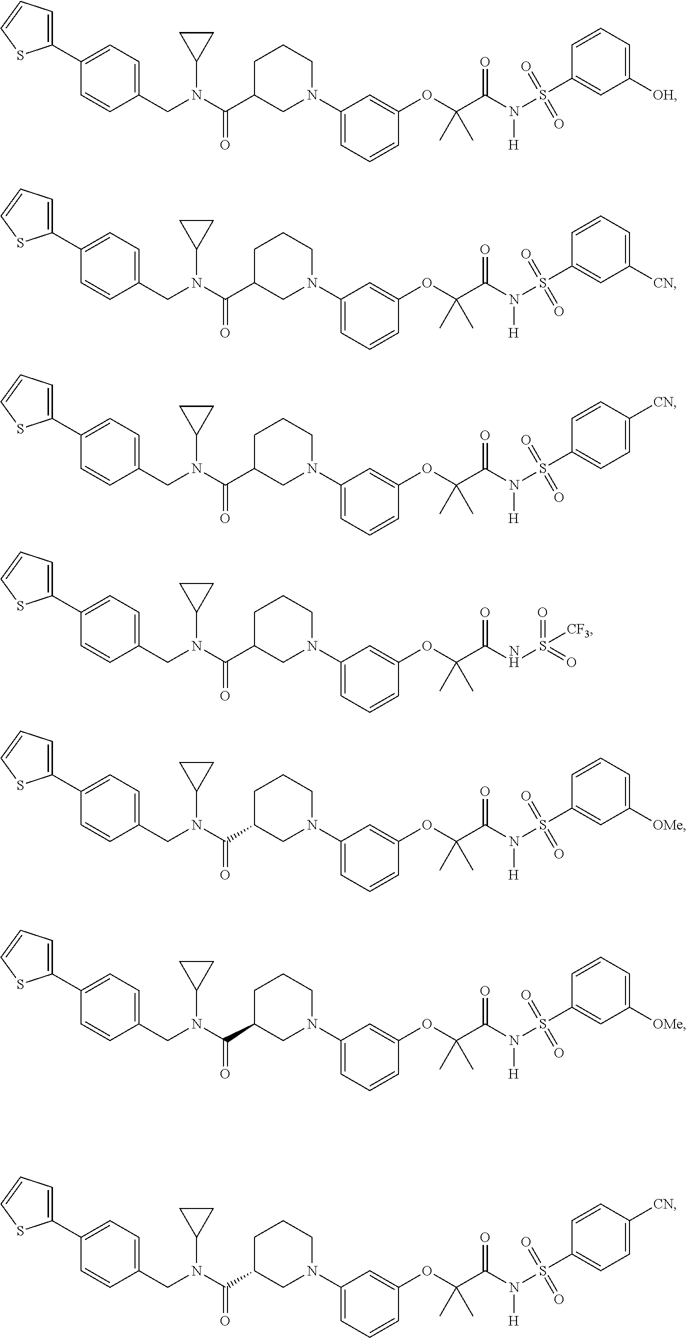 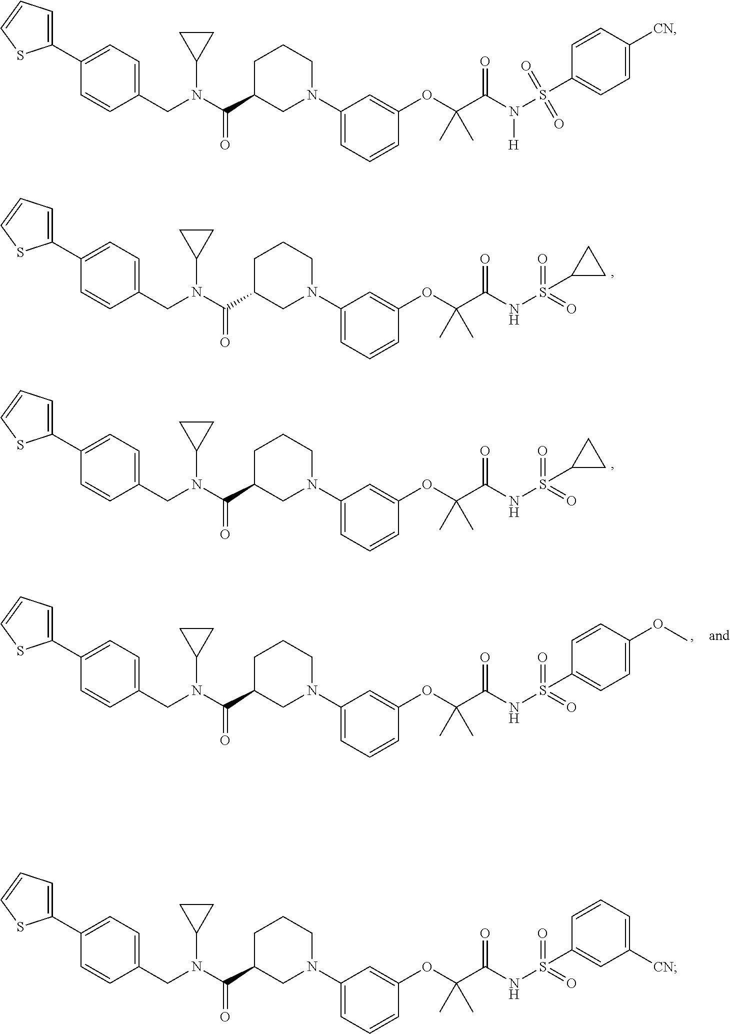 [SMILES: CC(C)(Oc1cccc(N2CCC[C@H](C(=O)N(Cc3ccc(-c4cccs4)cc3)C3CC3)C2)c1)C(=O)NS(=O)(=O)c1ccc(C#N)cc1 ; CC(C)(Oc1cccc(N2CCC[C@@H](C(=O)N(Cc3ccc(-c4cccs4)cc3)C3CC3)C2)c1)C(=O)NS(=O)(=O)C1CC1 ; CC(C)(Oc1cccc(N2CCC[C@H](C(=O)N(Cc3ccc(-c4cccs4)cc3)C3CC3)C2)c1)C(=O)NS(=O)(=O)C1CC1 ; COc1ccc(S(=O)(=O)NC(=O)C(C)(C)Oc2cccc(N3CCC[C@H](C(=O)N(Cc4ccc(-c5cccs5)cc4)C4CC4)C3)c2)cc1 ; CC(C)(Oc1cccc(N2CCC[C@H](C(=O)N(Cc3ccc(-c4cccs4)cc3)C3CC3)C2)c1)C(=O)NS(=O)(=O)c1cccc(C#N)c1]
or a pharmaceutically acceptable salt thereof.

16. (canceled)

17. A pharmaceutical composition comprising a compound of claim 1, or pharmaceutically acceptable salt thereof, and a pharmaceutically acceptable carrier.

18. A method of treating a disorder of uncontrolled cellular proliferation in a subject comprising administering to the subject a therapeutically effective amount of a compound of claim 1, or a pharmaceutically acceptable salt thereof.

19. The method of claim 18, wherein the compound inhibits β-catenin/B-cell lymphoma 9 complex and/or decreases cellular β-catenin levels.

20. (canceled)

21. The method of claim 18, wherein the subject is a human.

22. The method of claim 18, wherein the disorder is cancer.

23. The method of claim 22, wherein the cancer is selected from breast cancer, cervical cancer, gastrointestinal cancer, colorectal cancer, brain cancer, skin cancer, prostate cancer, ovarian cancer, thyroid cancer, testicular cancer, pancreatic cancer, endometrial cancer, melanoma, glioma, leukemia, lymphoma, chronic myeloproliferative disorder, myelodysplastic syndrome, myeloproliferative neoplasm, and plasma cell neoplasm (myeloma).

24. A method for inhibiting protein-protein interactions of β-catenin and B-cell lymphoma 9 in at least one cell comprising contacting the at least one cell with an effective amount of a compound of claim 1, or a pharmaceutically acceptable salt thereof.

25. A method for degrading β-catenin in at least one cell comprising contacting the at least one cell with an effective amount of a compound of claim 1, or a pharmaceutically acceptable salt thereof, wherein the compound comprises at least one linker conjugated to a PROTAC moiety.
